(19) United States
(12) Patent Application Publication
(10) Pub. No.: US 2022/0245798 A1
(43) Pub. Date: Aug. 4, 2022

(54) TISSUE STEM CELL COUNTING ALGORITHMS AND USES THEREOF

(71) Applicant: Asymmetrex LLC, Boston, MA (US)

(72) Inventor: James L. SHERLEY, Boston, MA (US)

(73) Assignee: Asymmetrex LLC, Boston, MA (US)

(21) Appl. No.: 17/583,918

(22) Filed: Jan. 25, 2022

Related U.S. Application Data
(60) Provisional application No. 63/141,707, filed on Jan. 26, 2021.
(60) Provisional application No. 63/157,208, filed on Mar. 5, 2021.

(51) Int. Cl. G06T 7/00; G16H 10/60; G16H 50/20; G01N 33/483

(52) U.S. Cl. CPC G06T 7/0012; G16H 10/60; G06T 2207/30024; G01N 33/4833; G06T 2207/30242; G16H 50/20

(57) ABSTRACT

Tissue stem cells are responsible for the maintenance and regeneration of mammalian organs, tissues, and cells, including those of humans. Convenient methods for specific and accurate counting of human and animal stem cells are needed for a wide array of applications, including but not limited to cell research, medicine, stem cell medicine, gene therapy, pharmaceutical and biopharmaceutical drug development, cell and tissue biomanufacturing and bioengineering, and environmental toxicology. The invention is mathematical algorithms that can be used to compute the tissue stem cell-specific fraction of any cell preparation, including human, from the input of the cell population doubling time of a cell preparation during culture.

### CROSS-REFERENCE TO RELATED APPLICATIONS

This Application claims benefit under 35 U.S.C. § 119(e) of U.S. Provisional Application No. 63/141,707 filed Jan. 26, 2021 and U.S. Provisional Application No. 63/157,208 filed Mar. 5, 2021, the contents of both of which are incorporated herein by reference in their entireties.

### TECHNICAL FIELD

The technology described herein relates to methods and systems of determining the number of tissue stem cells in a heterogeneous population of cells and methods of treating a patient in need of a stem cell transplant and uses thereof.

### BACKGROUND

Much of cell biomanufacturing is focused on supply of high quality, therapeutic, human tissue stem cell products. Being able to quickly, accurately, and economically quantify tissue stem cells is essential for the best success of the cell biomanufacturing industry. Yet, currently, the human tissue stem cell biomanufacturing industry lacks an efficient and simple means to quantify its key optimization target. For more than 60 years, there has been no method for specific and accurate counting of human tissue stem cells. This crucial unmet need limits progress in stem cell medicine, the major user of tissue stem cell manufactured products.

### SUMMARY

The compositions and methods described herein are based, in part, on the discovery of utilizing innovative stem cell counting algorithms that provide a much faster means of tissue stem cell counts as compared with any previous methods.

In one aspect, provided herein is a method of determining the fraction of tissue stem cells present in a tissue sample, the method comprising:
(a) culturing a heterogeneous population of cells from a specific tissue comprising tissue stem cells, transiently amplifying committed progenitor cells and terminally differentiated non-dividing cells;
(b) serially passaging said population of cells wherein the total number of live and dead cells in the population of cells are counted;
(c) processing the total cell count data with a model to determine the stem cell fraction (SCF) of the population of cells throughout the periods of the serial passaging;
(d) calculating the population doubling times (PDT) for said tissue cells during non-saturated or non-confluent periods of the serial passaging; and
(e) quantifying the number of tissue stem cells in the subsequent tissue samples of the same type based on (d), without serial passaging.

In another aspect, provided herein is a method of treating a subject in need thereof with a population of stem cells, the method comprising:
(a) determining the stem cell fraction present in a tissue sample according to the method provided herein; and
(b) administering to the subject in need thereof a dosage of stem cells based on the stem cell fraction (SCF).

In one embodiment of any of the aspects, the PDT is determined by the following Formula A:

$$PDT = \frac{{t}_{2} - {t}_{1}}{ln ( \frac{{N}_{2}}{{N}_{1}} )} ln 2 , \qquad ( Formula A )$$

where t₁=initial starting time, t₂=final time, N₁=the initial cell number, and N₂=the final cell number.

In another embodiment of any of the aspects, the SCF is determined by one of the following derivative Formulas B-D:

SCF=PDT (mean quotient of SCF/PDT) (Formula B);

SCF=e^{-(mPDT+b)} (Formula C); or

SCF=PDTe^{(at\hat{ }3+bt\hat{ }2+ct+d)} (Formula D),

where t=time.

It is noted that CPD is a routine measure of the proliferative capacity of tissue cell preparations

In another embodiment of any of the aspects, the cells are passaged every 12 hours or more, 24 hours or more, 48 hours or more, 72 hours or more, or 96 hours or more.

In another embodiment of any of the aspects, the cells are passaged on an irregular schedule based on when they achieve saturation density or confluence.

In another embodiment of any of the aspects, a constant fraction of cells is serially passaged.

In another embodiment of any of the aspects, a constant number of cells are serially passaged.

In another embodiment of any of the aspects, the PDT is determined in vitro or by in vivo imaging techniques.

In another embodiment of any of the aspects, the PDT is determined by using a counting method selected from the group consisting of: counting cells, an absorbance assay, a turbidity assay, weighing cells, a fluorescent assay, and combinations thereof.

In another embodiment of any of the aspects, the cells are serially passaged until the number of total cells after two consecutive passages does not increase.

In another embodiment of any of the aspects, the processing time is determined by a computer processing system.

In another embodiment of any of the aspects, the cells are processed until a predetermined number of tissue stem cells are present in the sample.

In another embodiment of any of the aspects, the cells are a vertebrate population of cells.

In another embodiment of any of the aspects, the cells are a mammalian population of cells.

In another embodiment of any of the aspects, the cells are a human population of cells.

In another embodiment of any of the aspects, the culturing is selected from the group consisting of: 3-dimensional cell culture; suspension cell culture; adherent cell culture; microcarrier cell culture; and any combination thereof.

In another embodiment of any of the aspects, wherein the cells are cultured in normoxic conditions. In another embodiment of any of the aspects, wherein the cells are cultured in hypoxic conditions.

In another embodiment of any of the aspects, the method further comprises contacting the cells with an agent.

In another embodiment of any of the aspects, the tissue stem cells are separated from initial transiently amplifying committed progenitor cells and terminally differentiated non-dividing cells.

In another embodiment of any of the aspects, the tissue stem cells are used to treat an individual.

In another embodiment of any of the aspects, the method further comprises administering to a subject in need thereof an appropriate amount of stem cells based on the stem cell fraction (SCF).

In another embodiment of any of the aspects, the subject in need thereof has or is suspected of having a disease, disorder, or injury.

In another embodiment of any of the aspects, the disease, disorder, or injury is selected from the group consisting of: effects on organs and tissues such as, but not limited to: lungs, heart, blood vessels, blood, liver, pancreas, muscle, bones, joints, eyes, central and peripheral nervous systems.

In some embodiments of any of the aspects, the administered stem cells have been genetically modified, including gene editing. In some embodiments of any of the aspects, the gene modifications may include, but are not limited to, deleting gene sequences, inserting gene sequences, or editing gene sequences in the nuclear genome or the mitochondrial genomes. In some embodiments of any of the aspects, the genetically modified stem cells are administered as the therapeutic agent for reducing the signs and symptoms of diseases, disorders, and injuries or for providing cosmetic changes in an individual.

The methods provided herein enable the calculation of a tissue cell preparation's SCF (and equivalent stem cell-specific dosage) by inputting its PDT into a defined kinetic stem cell (KSC) counting algorithm. Interestingly, the method provided herein can be used to determine the exponential decay half-life of the SCF for a cultured tissue cell preparation containing tissue stem cells.

Without wishing to be bound by a theory, when the initial SCF of a primary tissue cell preparation is known, the SCF half-life (SCHL) makes it possible to calculate the SCF fraction of the cell preparation at any time in future cultures based on knowing the number of CPDs (cumulative population doubling). Accordingly, in another aspect provided herein is method for determining SCF at a future CPD (SCF_{CPD}). The SCF at a future CPD is determined by the following Formula E:

SCF_{CPD}, SCF at any future CPD,=SCF₀ X e^{-(ln2/HL)CPD} (Formula E),

where SCF₀=initial SCF and HL=half-life.

### BRIEF DESCRIPTION OF THE DRAWINGS

This application file contains at least one drawing executed in color. Copies of this patent application publication with color drawings will be provided by the Office upon request and payment of the necessary fee.

FIG. 1 demonstrates cell kinetics model factors underpinning the TORTOISE Test™. Bivalent cells, stem cells; uniform circles, transiently amplifying, committed progenitor cells; uniform squares, terminally differentiated cells. Superscript m identifies measured input factors. Superscript d identifies factors discovered using the TORTOISE Test™ RIFS (Random Input Factor Search) software. FR_NS, stem cell fraction (SCF).

FIGS. 2A-2F demonstrate the deconstruction of PSCK (Probabilistic Stem Cell Kinetics) simulation data to determine stem cell-specific number and cell kinetics. FIG. 2A. Replicate (n=6) experimental CPD (cumulative population doubling) data from serial passage of pre-senescent human lung mesenchymal cells. FIG. 2B. PSCK simulation of human lung cell culture serial passage using input factors determined by RIFS computation. The black line is the mean CPD trace from the experimental data show. The black line traces the mean CPD values n in FIG. 2A. The root mean squared error (RMSE) of the simulation compared to the data was 0.19±0.11 (p=0.024). FIG. 2C. Examples of cell type-specific cell kinetics output from PSCK-RIFS computations. Shown are the deconstructed outputs for the individualized cell kinetics of stem cells (NS, blue, middle y-axis, max=5%), total transiently amplifying progenitor cells and differentiated cells (NT, green, right y-axis, max=105%), and terminally-arrested differentiated cells (NT-Terminal, red, left y-axis, max=102.5%) in terms of their percent cellularity in the serially passaged cultures. FIG. 2D-2F. Use of PSCK-RIFS to determine the specific stem cell number and kinetics in serial cultures from different human tissues. The total number of viable cells input at the start of each culture (0 hour) was 325,000 (FIG. 2D, expanded liver stem cells), 65,000 (FIG. 2E, HSCs in CD34⁺-selected bone marrow cells), and 375,000 (FIG. 2F, MSCs from primary bone marrow). Note the detection of symmetric self-renewal by liver stem cells (FIG. 2D, inclined phases) between passages (vertical lines), but not for HSCs or MSCs (FIGS. 2E and 2F, respectively, flat phases). This important biological distinction is quantified by the symmetric self-renewal rate, RS, (See in FIG. 1).

FIGS. 3A-3F show the defining population doubling time (PDT):stem cell fraction (SCF) algorithms. Examples of the SCF output (FIG. 3A; y-axis, % stem cells; x-axis, days) and total cell number output (FIG. 3B; y-axis, cell number; x-axis, days) for CD34⁺ cord blood (CB) cells. FIGS. 3C and 3E, examples of the derived plots of SCF versus PDT determined from corresponding total cell number data of CD34⁺ cord blood cells (derived from data in A and B) and lung mesenchymal cells, respectively. FIG. 3D and 3F, mathematical linearization of the data in C and E, respectively, to determine algorithms (F) that define mathematical relationships between PDT and SCF.

FIG. 4 shows probabilistic stem cell kinetics (PSCK) cellular model for cell production by adult tissue stem cells in culture based on tissue stem cell asymmetric self-renewal. In cell culture, adult tissue stem cells (bivalent circles) exist in one of four probabilistic cell kinetics states that are postulated to be extensions of analogous functions in tissues [10, 12, 13]. Q, quiescence, the state of reversible cell division arrest. D, the state of cell death. S, the state of symmetric self-renewal that produces two stem cells. A, the state of asymmetric self-renewal, which maintains a constant level of tissue stem cells while simultaneously producing committed progenitor cells (solid circles) that produce differentiated cell lineages that often end with terminally-arrested, non-dividing mature cells (squares). Generally, in tissues in vivo and in cell culture ex vivo, the probability of the A state is significantly greater than the probability of the S state. In vivo, this relationship maintains homeostatic tissue cell kinetics. Ex vivo during serial cell culture, the same relationship is predicted to cause a reduction in tissue stem cell fraction by cellular dilution.

FIGS. 5A-5C demonstrate one example of PSCK simulation of serial culture cumulative population doubling data. FIG. 5A. Replicate (six) experimental CPD data from serial passage of pre-senescent human WI-38 lung fibroblasts. The black line traces the mean CPD values. FIG. 5B. PSCK simulation of WI-38 lung culture serial passage using input factors determined by RIFS computation. The black line is the mean CPD trace from the experimental data shown in 5A. FIG. 5C. Comparison of the PSCK-simulated mean CPD (red trace) to the experimental mean CPD data (black trace). The root mean squared error of the simulation compared to the experimental data was 0.19±0.11 (p=0.024). Data also included in FIGS. 2A and 2B.

FIG. 6 demonstrates another example of cell type-specific cell kinetics output from PSCK-RIFS computations. Shown are the computed outputs for the individualized cell kinetics of stem cells (NS, blue, middle y-axis), total transiently amplifying progenitor cells plus terminally-arrested differentiated cells (NT, green, right y-axis), and terminally-arrested differentiated cells alone (NT-Terminal, red, left y-axis) in terms of their percentage cellularity in serially passaged WI-38 cultures. Note that the difference between the green trace and the red trace corresponds to transiently amplifying committed progenitor cells specifically. Outputs were derived from the simulation data presented in FIG. 5. Data also included in FIG. 2C.

FIGS. 7A-7C demonstrates the use of PSCK-RIFS to compute the specific stem cell number and kinetics during the serial culture of cells derived from different human tissues. The cell kinetics factors determined from respective simulations like the example shown in FIGS. 5A-5C were used to compute the number of tissue stem cells as a function of days of serial culture passage. The total number of viable cells input at the start of each culture (0 hour) was 325,000 (A, expanded liver stem cells [25]), 65,000 (B, HSCs in CD34⁺-selected bone marrow cells), and 375,000 (C, MSCs from primary bone marrow [24]). Note the detection of symmetric self-renewal by liver stem cells (A, inclined phases) between passages (perpendicular lines), but not for bone marrow-derived HSCs or MSCs (B and C, respectively, flat phases). This important biological distinction is quantified by the symmetric self-renewal rate, RS, (see Table 2). Data also included in FIGS. 2D-2F.

FIGS. 8A and 8B show ex vivo asymmetric self-renewal kinetics by tissue stem cells can explain the density dependence of serially passaged mouse embryo fibroblast cultures. FIG. 8A. Data reproduced with permission from Todaro and Green [18] (©1963 TODARO et al. Originally published in J. CELL BIOL. available on the world wide web at https://<doi.org/10.1083/jcb.17.2.299>) showing the increase in cumulative population doublings (generations) for serially passaged primary mouse embryo fibroblast cultures associated with the indicated increases in the number of cells transferred at 3 day intervals. FIG. 8B. After development of a PSCK simulation to approximate the data for the lowest transferred cell number (1×10⁵ cells), the subsequent simulations were developed after changing only to the indicated input number for cells transferred in the simulation.

FIG. 9 demonstrates TORTOISE Test™ software quantification of the number of HSCs in serial cultures of whole, unfractionated human CB cells. The red and blue traces correspond to computations for the specific HSC counts (NS) of the two independent duplicate samples of CB cells. The vertical drops in HSC number indicate the one third split of cultures when passaged every 72 hours.

FIGS. 10A and 10B show PDT:SCF stem cell counting algorithm discovery for HSCs in whole, unfractionated human umbilical CB. FIGS. 10A and 10B are the results of TORTOISE Test™ software analyses of data from the two independent, duplicate serial cultures of CB cells. The indicated algorithm function, ƒ, requires only simple PDT data from less than 3 days of culture to determine the initial HSC fraction of cultured CB samples.

FIG. 11 shows the generation of CPD data by serial culture.

FIGS. 12A and 12B show RIFS software search analyses of PSCK factor sets. FIG. 12A shows a trace of the software's search for simulations with the best quality scores that define how well simulations match experimental CPD data. FIG. 12B is a frequency distribution of the quality scores determined for the search presented in FIG. 12A. Arrow, example of the best (lowest) quality score defined in the distribution.

FIG. 13 shows an exemplary method for evaluating the stem cell fraction, according to an embodiment of the present disclosure.

FIGS. 14A and 14B shows SCF half-life graphs for human cord blood hematopoietic stem cells (FIG. 14A) and human neonatal hepatic stem cells (FIG. 14B).

FIGS. 15A-15C show KSC counting TORTOISE Test™ software analysis of human CD34⁺-selected UCB cells. The data from duplicate parallel serial cultures were evaluated. FIG. 15A. Input cell count data for the KSC counting software analysis. Circles, right y-axis, experimental mean total cell number at each passage of cell culture, based on the transfer of 1/10 of culture cells approximately every 3 days. Squares, left y-axis, mean CPD data derived from mathematical transformation of the mean total cell number data. FIG. 15B. The PSCK module of the TORTOISE Test™ software was used to compute 10 independent simulations (Note: some simulations are coincident) of the mean CPD data (black squares; same as in FIG. 15A) using input initial cell kinetics factors that were randomly-selected within the statistical ranges defined by the TORTOISE Test™ KSC counting software (See Table 6). FIG. 15C. The randomly-selected initial cell kinetics factors for one of the simulations in B (fRMSE=0.042) were used to compute respective cell kinetics profiles for HSCs (blue), CPCs (red), and terminally-arrested cells (green) during serial passaging.

FIGS. 16A-16F show rabbit PDT algorithms for rapid determination of HSC fraction. FIG. 16A-16F. Each panel provides a graphical representation of the respective KSC counting PDT algorithm, f(PDT:SCF), that can be used to calculate the HSC fraction (SCF) of the indicated commercial human hematopoietic cell preparation and culture medium conditions by input of only the day of serial culture and the preceeding 72-hour PDT of the culture. FIG. 16A-16E. Algorithms were defined by RABBIT Count™ software analyses of experimental CPD data from cultures maintained in the same culture medium (See Materials and Methods.) and passaged with a 1:10 split basis approximately every 3 days. FIG. 16F. Cells were cultured in the same medium as A-E, but instead passaged with a 1:5 split basis approximately every 3 days. 34, CD34⁺-selected fraction.

FIGS. 17A-17F show rabbit CPD algorithms for rapid determination of HSC fraction. Each panel provides a graphical representation of the respective KSC counting CPD algorithm that can be used to calculate the HSC fraction (SCF) of the indicated commercial human hematopoietic cell preparation and culture medium conditions by input of only the preparations number of CPDs. FIGS. 17A-17E. Algorithms were defined by RABBIT Count software analyses of experimental CPD data from cultures maintained in the same culture medium (See Materials and Methods.) and passaged with a 1:10 split basis approximately every 3 days. FIG. 17F. Cells were cultured in the same medium as A-E, but instead passaged with a 1:5 split basis approximately every 3 days. 34, CD34⁺-selected fraction. SCFHL, SCF half-life.

FIG. 18 shows validation analysis for the ability of a rapid-counting Rabbit PDT algorithm to accurately calculate the HSC fraction of CD34⁺-selected UCB cell preparations. Shown is an evaluation of the ability of a primary Rabbit PDT algorithm to accurately calculate the HSC fraction (SCF) of a parallel independent culture of the same type. Data from the parallel serial culture analyses described in FIG. 16E and FIG. 16F, for respective 1:10 versus 1:5 split basis, were used for the analysis. Experimental mean PDT data from the 1:5 split-basis study were input into the Rabbit algorithm developed with the 1:10 split-basis study data to calculate HSC fraction data for the 1:5 split-basis study. The natural logarithm of the HSC fraction data calculated with the Rabbit algorithm (1:10 Rabbit) was plotted versus the natural logarithm of the HSC fraction data determined by the primary TORTOISE Test™ KSC counting software analysis of the 1:5 split-basis study data (1:5 Tortoise).

FIG. 19 shows analyses of the mean HSC fraction determinations for three independent P2 secondary KSC counting analyses. Triplicate parallel serial cultures were performed for three independent vials of cryopreserved human CD34⁺-selected UCB cells. The cells thawed for these passage 2 (P2) secondary cultures were derived from parallel cultures of the same cultures described in FIG. 15 (See Materials and Methods). The natural logarithm of the mean HSC fraction (SCF) is plotted versus days of serial passage. The cell count data from the three respective sets of triplicate P2 secondary serial cultures were used to develop independent KSC counting software determinations of their HSC fractions (SCF) with serial passage. The natural logarithm of the mean HSC fraction for the three TORTOISE Test™ KSC counting analysis is plotted versus days of passage. Error bars=95% confidence intervals.

FIGS. 20A-20D show validation analysis of the ability of a 1st degree rapid-counting Rabbit PDT algorithm to accurately compute the HSC fraction of secondary serial cultures. FIGS. 20A-20C. Triplicate parallel serial cultures were performed for three independent vials of cryopreserved human CD34⁺-selected UCB cells (Vials 1-3; same as in FIG. 19). The cells thawed for these passage 2 (P2) secondary cultures were derived from parallel cultures of the same cultures described in FIG. 15 and used to develop the primary Rabbit PDT algorithm in FIG. 16E (1stD Rabbit). The cell count data from the three respective sets of triplicate P2 secondary serial cultures were used to develop independent TORTOISE Test™ KSC counting software determinations of their HSC fractions (SCF) with passage. These data were evaluated for correlation with HSC fraction calculations made by inputting the respective experimental mean 72-hour PDT data of the P2 secondary serial cultures into the 1st degree Rabbit PDT algorithm (FIG. 16E). The natural logarithms of the SCF data were compared. FIG. 20D. The independent data sets in FIGS. 20A-20C were combined for an overall correlation estimation of the accuracy of the 1^{st} degree rapid-counting Rabbit PDT algorithm for determination of the HSC fraction of secondary cultures of the same type.

### DETAILED DESCRIPTION

The compositions and methods described herein, in part, utilize the discovery of innovative stem cell counting algorithms that provide tissue stem cell counts more rapidly than previous methods. This can in one embodiment be just a few days, e.g., 3-4 days.

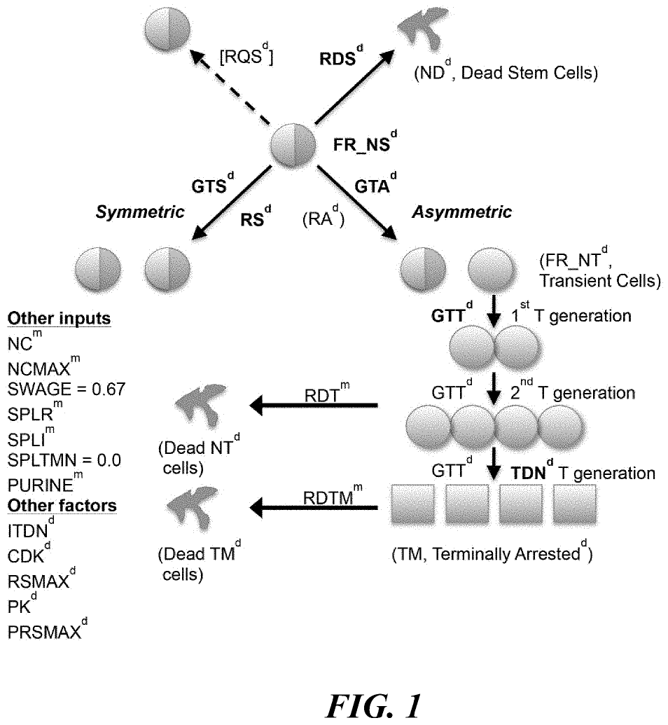

Provided herein is a method of determining the fraction of tissue stem cells present in a biological sample. The fundamental strategy for determining the number of tissue stem cells in a sample is depicted in FIGS. 1, 11, and 13.

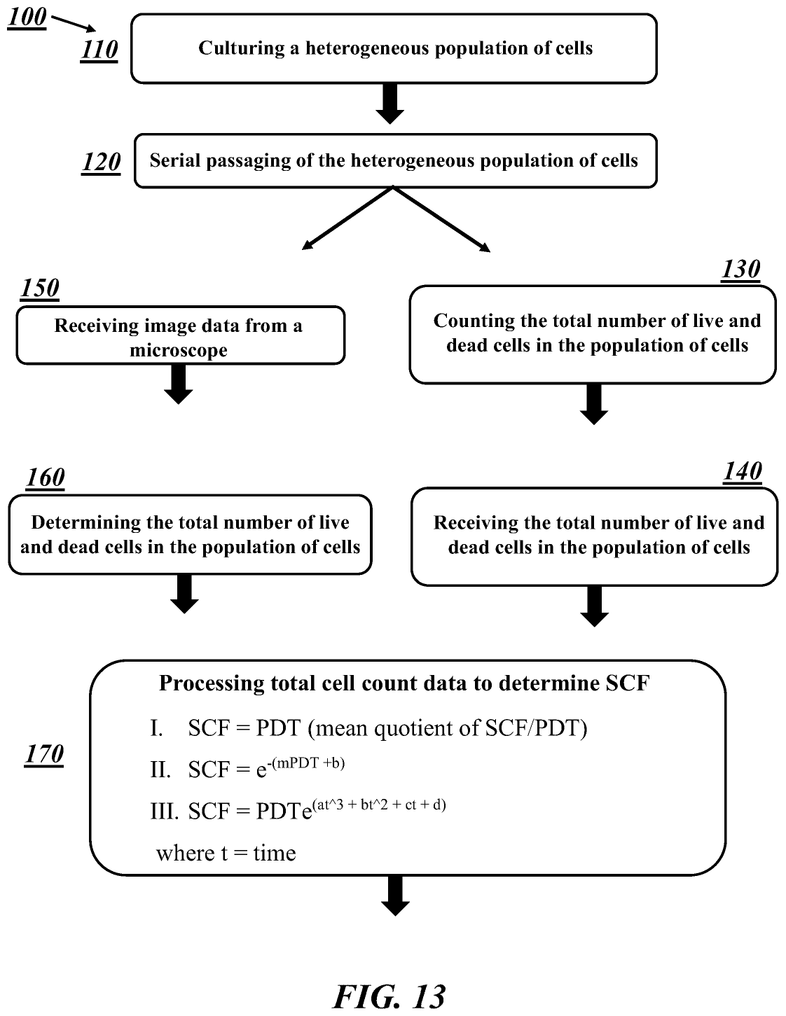

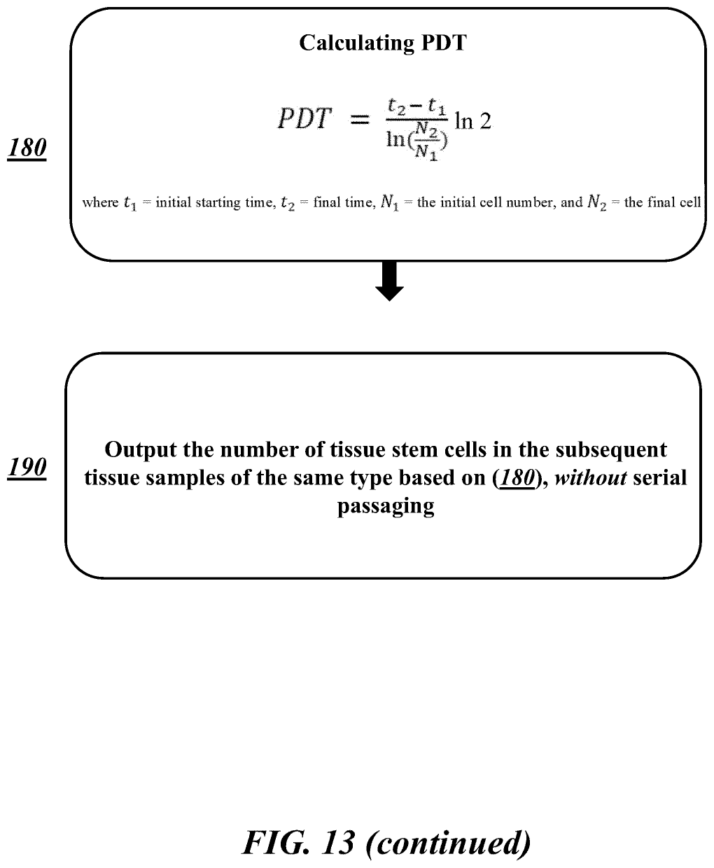

FIG. 13 shows an exemplary method (100) for evaluating the stem cell fraction present in a tissue sample according to an embodiment of the present disclosure. Method 100 begins by culturing a heterogeneous population of cells comprising tissue stem cells, transiently amplifying committed progenitor cells and terminally differentiated non-dividing cells (110). The cells are then serially passaged (120). In some embodiments, the cells are passaged until the number of total cells after two consecutive passages does not increase. The cells can be counted using any method known in the art (140) or image data from a microscope can be used to determine the total number of live and dead cells in the population of cells (150-160). In some embodiments, the total number of live and dead cells is determined by a computer processing system. The total cell count data is processed with a model to determine the stem cell fraction (SCF) of the population of cells throughout the periods of the serial passaging (170). In some embodiments, SCF is determined by one or more of the following equations selected from: I. SCF=PDT (mean quotient of SCF/PDT); II. SCF=e\hat{ }-(mPDT+b); and III. SCF=PDTe\hat{ }(at\hat{ }3+bt\hat{ }2+ct+d), where t=time. The population doubling times (PDT) for said tissue cells are calculated during non-saturated or non-confluent periods of the serial passaging (180). In some embodiments, the processing time is determined by a computer processing system. The number of tissue stem cells in the subsequent tissue samples are quantified based on the same type based in (180), without serial passaging (190). In some embodiments, the number of tissue stem cells is output by the computer processing system.

### Cell Compositions and Cell Culture

In some embodiments, the biological sample is a tissue sample and/or a heterogeneous population of cells.

The methods and systems provided herein comprise a step of culturing a heterogeneous population of cells from a specific tissue comprising tissue stem cells, and transiently amplifying committed progenitor cells and terminally differentiated non-dividing cells. In some embodiments of any of the aspects, the cells are a vertebrate population of cells. In some embodiments of any of the aspects, the cells are mammalian or human cells.

The heterogeneous cell population can be isolated from tissue of an adult mammal, preferably a human. Cells can be obtained from donor tissue, such as donor skin or other organs, by dissociation of individual cells from the connecting extracellular matrix of the tissue. Tissue is removed using a sterile procedure, and the cells are dissociated using any method known in the art including treatment with enzymes such as trypsin, collagenase, and the like, or by using physical methods of dissociation such as with a blunt instrument. The heterogeneous cell population may also be obtained from bodily fluids; including, but not limited to, blood, umbilical cord blood, amniotic fluid, spinal fluid, pleural fluid, and lymphatic fluid.

In some embodiments, the heterogeneous population of cells is obtained from organ tissue. Tissue can be obtained from any organ, including but not limited to: organs of the musculoskeletal skeletal system, e.g. bone, cartilage, fibrous joints, cartilaginous joints, synovial joint, muscle, tendon, or diaphragm; organs of the cardiovascular system, e.g. artery, vein, lymphatic vessel, or heart; organs of the lymphatic system, e.g. primary (bone marrow, thymus), secondary (spleen and lymph node), CNS equivalent (cerebral spinal system); organs of the nervous system, e.g. brain, spinal cord, nerve; organs of the sensory system, e.g. ear, cochlea, eye; organs of the integumentary system, e.g. skin, subcutaneous tissue, breast (mammary gland), hair; organs of the immune system, e.g. myeloid (myeloid immune system) or lymphoid (lymphoid immune system); organs of the respiratory system, e.g. upper (e.g. nose, nasopharynx, larynx) or lower system (e.g. trachea, bronchus, lung); organs of the digestive system, e.g. mouth (salivary gland, tongue), upper gastrointestinal (GI; oropharynx laryngopharynx, esophagus, stomach), lower GI (e.g. small intestine, appendix, colon, rectum, anus) or accessory GI (e.g. liver, biliary tract, pancreas); organs of the urinary system, e.g. genitourinary system: e.g. kidney, ureter, bladder, and urethra; organs of the reproductive system, female (uterus, vagina, vulva, ovary, placenta) male (scrotum, penis, prostate, testicle, seminal vesicle); organs of the endocrine system, e.g. pituitary, pineal gland, thyroid, parathyroid, adrenal, or islets of Langerhans; and perinatal organs and tissues, e.g., umbilical cord, amniotic membrane, or placenta. Cells can be of the mesoderm, endoderm, or ectoderm origin.

Also useful in methods provided herein are cell culture systems that contain a heterogeneous population of cells including stem cells, transient cells, and terminally differentiated cells. Such systems are known to those of skill in the art and include, but are not limited to those described in, U.S. patents and publications 20140193910; U.S. Pat. Nos. 8,759,098; 8,404,481; 7,883,891; 7,867,712; 7,824,912; 7,655,465; 7,645,610, and 20030133918; which are herein incorporated by reference in their entirety.

Somatic stem cells, also known as adult stem cells, are derived from tissues of a fetal or neonatal organism or from a post-natal adult organism, in contrast to other sources of stem cells such as embryonic stem cells, which may originate from a variety of sources of embryonic, pre-fetal tissues. Somatic stem cells are particularly attractive for a range of therapies in light of the ongoing controversies surrounding the use of embryonic stem cells. An adult stem cell is physiologically and phenotypically distinct from an embryonic stem cell not only in markers it does or does not express relative to an embryonic stem cell, but also by the presence of epigenetic differences, e.g. differences in DNA methylation patterns, and as provided herein, the cell kinetics properties of the cells, i.e., their patterns of division with respect to the cells that they produce. The properties of somatic stem cells are described, e.g., in U.S. Pat. Nos. 7,883,891 B2 and 7,867,712 B2, the contents of each of which is incorporated herein by reference in their entireties.

In some embodiments of any of the aspects, the cells are cultured in a standard suspension culture or an adherent cell culture. In some embodiments of any of the aspects, cells are cultured in a 3-dimensional cell culture or a microcarrier cell culture. The type of cell culture method is can be determined by one of skill in the art for a given application of the counting method provided herein.

Any medium can be used that is capable of supporting cell growth. For example, the medium can include but is not limited to: HEM, DMEM, RPMI, F-12, and the like. The medium can contain supplements or agents which are required for cellular metabolism such as glutamine and other amino acids, vitamins, minerals and useful proteins such as transferrin and the like. Medium may also contain antibiotics to prevent contamination with yeast, bacteria and fungi such as penicillin, streptomycin, gentamicin and the like. In some cases, the medium may contain serum derived from bovine, equine, chicken and the like. Serum can contain xanthine, hypoxanthine, or other compounds that enhance guanine nucleotide biosynthesis, although generally at levels below the effective concentration to suppress asymmetric cell kinetics. In one embodiment, for the cell culture the medium and serum contain levels below the effective concentration to suppress asymmetric cell kinetics. In some embodiments, the culture medium is a defined culture medium comprising a mixture of DMEM, F12, and a defined hormone and salt mixture.

The culture medium can be supplemented with a proliferation-inducing growth factor(s) or agents as defined herein. As used herein, the term “growth factor” refers to a protein, peptide or other biological molecule having a growth, proliferative, differentiating, or trophic effect on stem cells or other cell types. Growth factors that may be used include any trophic factor that promotes cells to proliferate, including any molecule that binds to a receptor on the surface of the cell to exert a trophic, or growth-inducing effect on the cell. Examples of proliferation-inducing growth factors include, but are not limited to, EGF, amphiregulin, acidic fibroblast growth factor (aFGF or FGF-1), basic fibroblast growth factor (bFGF or FGF-2), transforming growth factor alpha (TGF.alpha.), and combinations thereof. Growth factors are usually added to the culture medium at concentrations ranging between about 1 fg/ml to 1 mg/ml. Concentrations between about 1 to 100 ng/ml are usually sufficient. Simple titration experiments can be easily performed to determine the optimal concentration of a particular growth factor. In one preferred embodiment, epidermal growth factor is used. In addition to proliferation-inducing growth factors, other growth factors may be added to the culture medium that influence proliferation and differentiation of the cells including NGF, platelet-derived growth factor (PDGF), thyrotropin releasing hormone (TRH), transforming growth factor betas (TGFβs), insulin-like growth factor (IGF-1) and the like.

Conditions for culturing should be close to physiological conditions. The pH of the culture medium should be close to physiological pH, preferably between pH 6-8, e.g., between about pH 7 to 7.8, with pH 7.4 being most preferred. Physiological temperatures range between about 30° C. to 40° C. Cells are preferably cultured at temperatures between about 32° C. to about 38° C., and more preferably between about 35° C. to about 37° C. Culture is often performed in ambient oxygen, but it may also be performed with regulated oxygen concentration, including hypoxic conditions.

The methods provided herein comprise a step of serially passaging the heterogeneous population of cells described herein wherein the total number of live and dead cells in the population of cells are counted. Methods of passaging cells are known in the art, see, e.g., US Pat Pg 2003/0133918 A1, the contents of which is incorporated herein by reference in its entirety.

Conventional serial passaging involves growing a cell culture until the culture vessel is replete with cells, e.g., greater than 70%, greater than 80%, or more confluency for adherent cultures or at saturation when nutrients become limiting in suspension cell cultures. Confluence of an adherent cell culture occurs when the culture vessel's maximum cell capacity is reached before dilution with additional cell culture medium and adherent surface. Saturation of a suspension cell culture occurs when the rate of cell proliferation begins to decrease before dilution with additional cell culture medium. When replete, the cells are harvested; and a fixed fraction of the harvested cells is transferred to a new culture vessel. The new culture is allowed to grow until replete again, and the dilution process is performed again. There are well known examples of such serial passaging schedules for both human cells and rodent cells. See, e.g., Hayflick, L. (1965) “The Limited In Vitro Lifetime of Human Diploid Cell Strains,” Exp. Cell Res. 37, 614-636; and Todaro, G. J. and Green, H. (1963) “Quantitative Studies of the Growth of Mouse Embryo Cells in Culture and Their Development into Established Lines,” J. Cell Biol. 17, 299-313, the contents of each of which are incorporated herein by reference in their entireties.

In the case of human tissue cell cultures, this serial process inevitably leads to a complete stoppage in new cell production. At the endpoint, the cultures are predicted to contain only terminal cells. This outcome results from first dilution of tissue stem cell number to zero, followed by completion of the remaining transient cells differentiation and production of terminal cells.

In some embodiments of any of the aspects, the cells described herein are passaged every 8 hours or more, 12 hours or more, 16 hours or more, 20 hours or more, 24 hours or more, 36 hours or more, 48 hours or more, 72 hours or more, or 96 hours or more, irrespective of their state of repletion.

In some embodiments of any of the aspects, a constant fraction of cells is serially passaged irrespective of their state of repletion. In some embodiments of any of the aspects, a constant number of cells are serially passaged, irrespective of their state of repletion. In some embodiments of any of the aspects, the cells are serially passaged until the number of total cells after two consecutive passages does not increase.

In some embodiments of any of the aspects, the cells are passaged on an irregular schedule based on when they achieve saturation density or confluence.

In some embodiments of any of the aspects, the method provided herein further comprises contacting the cells with an agent. The term “agent” refers to any entity to be administered to or contacted with a cell, tissue, organ or subject that is normally not present or not present at the levels being administered to the cell, tissue, organ, or subject. Agents can be selected from a group comprising: chemicals; small molecules; nucleic acids; nucleic acid analogues; proteins; peptides; peptidomimetics; peptide derivatives; peptide analogs; aptamers; antibodies; intrabodies; biological macromolecules; or functional fragments thereof.

In some embodiments of any of the aspects, all aspects of the system are controlled by a central processor (computer).

### Methods of Determining the Stem Cell Fraction (SCF) and Population Doubling Time (PDT)

The methods provided herein comprise a step of processing the number of stem cells in the heterogeneous population of cells. The total cell count data can be processed using a model provided herein to determine the stem cell fraction (SCF) of the population of cells throughout the periods of the serial passaging. This can be achieved by calculating the population doubling times (PDT) for the tissue cells during non-saturated or non-confluent periods of the serial passaging; and quantifying the number of tissue stem cells in the subsequent tissue samples of the same type without serial passaging.

In some embodiments of any of the aspects, the cells are processed until a predetermined number of tissue stem cells are present in the sample.

In some embodiments of any of the aspects, the PDT is determined by the following formula:

$$PDT = \frac{{t}_{2} - {t}_{1}}{ln ( \frac{{N}_{2}}{{N}_{1}} )} ln 2$$

where t₁=initial starting time, t₂=final time, N₁=the initial cell number, and N₂=the final cell number.

In some embodiments of any of the aspects, the SCF is determined by the following derivative formulas:

I. SCF=PDT (mean quotient of SCF/PDT)

II. SCF=e^{-(mPDT+b)}

SCF=PDTe^{(at\hat{ }3+bt\hat{ }2+ct+d)}

where t=time

In some embodiments of any of the aspects, the PDT is determined by using a counting method selected from the group consisting of: counting cells, an absorbance assay, a turbidity assay, weighing cells, a fluorescent assay, and combinations thereof. The counting method can be manual or automated, e.g, using a commercially available counter, e.g., Vi-Cell™ cell counter (Beckman Coulter®). Cell counts can be determined in vitro or by in vivo imaging techniques. Methods of determining the number of viable cells in the population are known in the art. For example, cell viability kits are commercially available, e.g., the LIVE/DEAD Viability/Cytotoxicity Kit™ for mammalian cells (ThermoFisher Scientific®—Catalog #L3224).

In some embodiments of any of the aspects, the processing time is determined by a computer processing system. Implementation of the methods provided herein using computer processing are discussed further below.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Methods of Isolating Adult Tissue Stem Cells

In some embodiments of any of the aspects, the method further comprises separating and/or isolating the tissue stem cells from initial transiently amplifying committed progenitor cells and terminally differentiated non-dividing cells. Methods of separating and isolating cells from a heterogeneous population of cells are known in the art, e.g., Fluorescence-activated cell sorting (FACS), Density Gradient Centrifugation, Immunodensity Cell Separation, Microfluidic Cell Separation, Immunomagnetic Cell Separation. Additional methods of isolating specific cell types are described, e.g., in Aijaz, A. et al. Biomanufacturing for clinically advanced cell therapies. Nat. Biomed. Eng. 2, 362-376 (2018); and Jiang Y. et al. Multipotent progenitor cells can be isolated from postnatal murine bone marrow, muscle, and brain. Exp. Hematol. 2002; 30: 896-904; the contents of each of which are incorporated herein by reference in their entireties.

In some embodiments of any of the aspects, the isolated tissue stem cells are administered to a subject in need thereof.

### Methods of Treating a Subject in Need Thereof

In another aspect, provided herein is a method of treating a subject in need thereof with a population of stem cells, the method comprises: (a) determining the stem cell fraction (SCF) present in a tissue sample according to the method provided herein; and (b) administering to the subject in need thereof a dosage of stem cells based on the stem cell fraction (SCF).

In some embodiments of any of the aspects, the subject in need thereof has or is suspected of having a disease, disorder, or injury. Exemplary diseases, disorders, or injuries that may be treated in a subject with the cells provided herein include but are not limited to: lungs, heart, blood vessels, blood, liver, pancreas, muscle, bones, joints, eyes, central and peripheral nervous systems. Exemplary diseases and disorders include but are not limited to: neurodegenerative and neurological diseases, blood diseases, cardiovascular diseases, spinal cord injuries, diabetes, kidney disease, immunosuppressive diseases, cancer, liver diseases, lung diseases, muscular diseases, amilial hypercholesterolemia, polycystic kidney disease, neurofibromatosis, hereditary spherocytosis, Marfan syndrome, Huntington's disease; ALS, Sickle cell anemia; cystic fibrosis; Tay-Sachs disease; phenylketonuria; mucopolysaccharidoses; mucopolysaccharidoses; lysosomal acid lipase deficiency; glycogen storage diseases; galactosemia; Duchenne muscular dystrophy; and hemophilia.

In some embodiments, the tissue stem cells are isolated and administered to a subject based on the SCF and/or PDT using the algorithms provided herein. The exact amount of cells required to treat a subject in need thereof will vary depending on factors such as the type of disease, disorder, or injury being treated.

The term “effective amount” or “appropriate amount” as used herein refers to the amount of a population of tissue stem cells or their differentiated progeny needed to alleviate at least one or more symptoms of a disease or disorder, including but not limited to an injury, disease, or disorder. An “effective amount” relates to a sufficient amount of a composition to provide the desired effect, e.g., treat a subject having an infarct zone following myocardial infarction, enhance vascularization of a graft, repair a spinal cord injury, restore muscle function, increase the levels or activity of blood cells, etc. The term “therapeutically effective amount” therefore refers to an amount of cells or a composition such cells that is sufficient to promote a particular effect when administered to a typical subject, such as one who has, or is at risk for, a disease or disorder. An effective amount as used herein would also include an amount sufficient to prevent or delay the development of a symptom of the disease, alter the course of a disease symptom (for example but not limited to, slow the progression of a symptom of the disease), or reverse a symptom of the disease. In some embodiments of any of the aspects, the appropriate amount of stems cells based on the SCF permits the engraftment of stems cells into the organ or tissue being treated. The administering can be done by direct injection (e.g., directly administered to a target cell or tissue) to the subject in need thereof. Administering can be transient, local, or systemic.

In some embodiments of any of the aspects, the stem cells are administered to the subject in a transplant composition. In some embodiments of any of the aspects, the transplant composition comprises a pharmaceutically acceptable carrier. In some embodiments of any of the aspects, the stem cells or the transplant composition are administered in combination with a therapeutic agent or a plurality of agents.

In general, the compositions comprising the cells provided herein are administered as liquid suspension formulations including the cells in combination with the pharmaceutically acceptable carrier. One of skill in the art will recognize that a pharmaceutically acceptable carrier to be used in a transplant composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells as described herein using routine experimentation.

Transplant compositions can optionally contain additional bioactive ingredients that further promote the survival, engraftment or function of the administered cells or, optionally, the tissue, organ or subject to which the composition is administered. Examples include, but are not limited to growth factors, nutrients, analgesics, anti-inflammatories and small molecule drugs, such as kinase activators, among others.

Physiologically tolerable carriers for the suspension of cells for a transplant composition include sterile aqueous physiological saline solutions that contain no additional materials other than the cells, or that contain a buffer such as sodium phosphate at physiological pH value, such as phosphate-buffered saline. Still further, aqueous carriers can contain more than one buffer salt, as well as salts such as sodium and potassium chlorides, dextrose, polyethylene glycol and other solutes.

The choice of formulation of the cells provided herein will depend upon the specific composition used and the number of cells to be administered; such formulations can be adjusted by the skilled practitioner. However, as an example, where the composition is tissue stem cells in a pharmaceutically acceptable carrier, the composition can be a suspension of the cells in an appropriate buffer (e.g., saline buffer) at an effective concentration of cells per mL of solution. The formulation can also include cell nutrients, a simple sugar (e.g., for osmotic pressure regulation) or other components to maintain the viability of the cells. Alternatively, the formulation can comprise a scaffold, such as a biodegradable scaffold.

In some embodiments of any of the aspects, the administered stem cells have been genetically modified, including gene editing. In some embodiments of any of the aspects, the gene modifications may include, but are not limited to, deleting gene sequences, inserting gene sequences, or editing gene sequences in the nuclear genome or the mitochondrial genomes. In some embodiments of any of the aspects, the genetically modified stem cells are administered as the therapeutic agent for reducing the signs and symptoms of diseases, disorders, and injuries or for providing cosmetic changes in an individual. Methods of genetically modifying stem cells and their differentiated progeny are known in the art. See, e.g., in WO2015/013583A2; U.S. Pat. No. 10,640,789 B2; US Pg. No. US2019/0367948 A1; Rees et al. Nature Rev Genet. 19(12); 770-788 (2018) and Kopmor et al. Nature 533, 420-424 (2016), the contents of each of which are incorporated herein by reference in their entirety. One of ordinary skill in the art can design and test a genetically modified adult stem cells described herein.

Exemplary embodiments of the disclosure can be described by the following numbered embodiments:

Embodiment 1: A method for determining the fraction of tissue stem cells present in a tissue sample, the method comprising: (a) culturing a heterogeneous population of cells from a specific tissue comprising tissue stem cells, transiently amplifying committed progenitor cells and terminally differentiated non-dividing cells; (b) serially passaging said population of cells wherein the total number of live and dead cells in the population of cells are counted; (c) processing the total cell count data with a model to determine the stem cell fraction (SCF) of the population of cells throughout the periods of the serial passaging; (d) calculating the population doubling times (PDT) for said tissue cells during non-saturated or non-confluent periods of the serial passaging; and (e) quantifying the number of tissue stem cells in the subsequent tissue samples of the same type based on (d), without serial passaging.

Embodiment 2: The method according to embodiment 1, wherein the PDT is determined by the following Formula A:

$$PDT = \frac{{t}_{2} - {t}_{1}}{ln ( \frac{{N}_{2}}{{N}_{1}} )} ln 2 , \qquad ( Formula A )$$

where t₁=initial starting time, t₂=final time, N₁=the initial cell number, and N₂=the final cell number.

Embodiment 3: The method according to embodiment 1 or 2, wherein the SCF is determined by one of the following derivative Formulas B-D

SCF=PDT (mean quotient of SCF/PDT) (Formula B);

SCF=e^{-(mPDT+b)} (Formula C); or

SCF=PDTe^{(at\hat{ }3+bt\hat{ }2+ct+d)} (Formula D),

where t=time.

Embodiment 4: The method according to any one of embodiments 1-3, wherein the cells are passaged every 12 hours or more, 24 hours or more, 48 hours or more, 72 hours or more, or 96 hours or more.

Embodiment 5: The method according to any one of embodiments 1-4, wherein the cells are passaged on an irregular schedule based on when they achieve saturation density or confluence.

Embodiment 6: The method according to any one of embodiments 1-5, wherein a constant fraction of cells are serially passaged.

Embodiment 7: The method according to any one of embodiments 1-6, wherein a constant number of cells are serially passaged.

Embodiment 8: The method according to any one of embodiments 1-7, wherein the PDT is determined in vitro or by in vivo imaging techniques.

Embodiment 9: The method according to any one of embodiments 1-8, wherein the PDT is determined by using a counting method selected from the group consisting of: counting cells, an absorbance assay, a turbidity assay, weighing cells, a fluorescent assay, and combinations thereof.

Embodiment 10: The method according to any one of embodiments 1-9, wherein the cells are serially passaged until the number of total cells after two consecutive passages does not increase.

Embodiment 11: The method according to any one of embodiments 1-10, wherein the processing time is determined by a computer processing system.

Embodiment 12: The method according to any one of embodiments 1-11, wherein the cells are processed until a predetermined number of tissue stem cells are present in the sample.

Embodiment 13: The method according to any one of embodiments 1-12, wherein the cells are a vertebrate population of cells.

Embodiment 14: The method according to any one of embodiments 1-13, wherein the cells are a mammalian population of cells.

Embodiment 15: The method according to any one of embodiments 1-14, wherein the cells are a human population of cells.

Embodiment 16: The method according to any one of embodiments 1-15, wherein the culturing is selected from the group consisting of: 3-dimensional cell culture; suspension cell culture; adherent cell culture; microcarrier cell culture; and any combination thereof.

Embodiment 17: The method according to any one of embodiments 1-16, further comprising contacting the cells with an agent.

Embodiment 18: The method according to any one of embodiments 1-17, wherein the cells are cultured in hypoxic conditions.

Embodiment 19: The method according to any one of embodiments 1-18, wherein the tissue stem cells are separated from initial transiently amplifying committed progenitor cells and terminally differentiated non-dividing cells.

Embodiment 20: The method according to any one of embodiments 1-19, wherein the tissue stem cells are used to treat an individual.

Embodiment 21: The method according to any one of embodiments 1-20, further comprising administering to a subject in need thereof an appropriate amount of stem cells based on the stem cell fraction (SCF).

Embodiment 22: The method according to embodiment 21, wherein the stem cells have been genetically modified.

Embodiment 23: The method according to embodiment 21 or 22, wherein the genetic modification is selected from the group consisting of: deleting gene sequences, inserting gene sequences, editing gene sequences in the nuclear genome, editing sequences in mitochondrial genomes.

Embodiment 24: The method according to any one of embodiments 21-23, wherein the stem cells are administered as a therapeutic agent for reducing the signs and symptoms of diseases, disorders, and injuries or for providing cosmetic changes in an individual.

Embodiment 25: A method of treating a subject in need thereof with a population of stem cells, the method comprising: (a) determining the stem cell fraction present in a tissue sample according to the method according to any one of embodiments 1-24; and (b) administering to the subject in need thereof a dosage of stem cells based on the stem cell fraction (SCF).

Embodiment 26: The method according to embodiment 25, wherein the subject in need thereof has or is suspected of having a disease, disorder, or injury.

Embodiment 27: The method according to embodiment 25 or 26, wherein the disease, disorder, or injury is selected from the group consisting of effects on organs and tissues such as, but not limited to: lungs, heart, blood vessels, blood, liver, pancreas, muscle, bones, joints, eyes, central and peripheral nervous systems.

Embodiment 28: The method according to any one of embodiments 25-27, wherein the stem cells have been genetically modified.

Embodiment 29: The method according to any one of embodiments 25-28, wherein the genetic modification is selected from the group consisting of: deleting gene sequences, inserting gene sequences, editing gene sequences in the nuclear genome, editing sequences in mitochondrial genomes.

### Some Selected Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology and molecular biology can be found in Molecular Biology Of The Cell, 6^{th} ed. published by W. W. Norton & Company., 2014, (ISBN: 9780815344322); Karp's Cell and Molecular Biology 9th Edition, published by Wiley 2020, (ISBN: 1119598249); The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Genetics: Analysis of Genes and Genomes 9^{th} ed., published by Jones & Bartlett Publishers, 2014 (ISBN: 978-1284122930); Biology published by Pearson, 11^{th} ed. 2016, (ISBN: 0134093410); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of each of which are all incorporated by reference herein in their entireties.

As used herein the term “stem cell” refers to a cell that can self-renew and asymmetrically produce at least one differentiated cell type. The term “human stem cell” encompasses perinatal and postnatal human unipotent and multipotent tissue stem cells.

As used herein, the term “passage” refers to diluting the cell culture, whether it be on plates, or in suspension, and re-culturing (re-plating) the diluted cells. For example, for passage of cells, the cells are removed from their tissue culture dish (e.g. by treatment with trypsin) or flask and diluted so that they can be re-plated (on plates or in a flask) and allowed to continue to grow.

The term “biomarker” as used herein is used to describe a characteristic and/or phenotypic indicator of a cell. Biomarkers can be used for selection of cells comprising characteristics of interest and can vary with specific cells. Biomarkers are morphological, structural, functional or biochemical (enzymatic) characteristics of particular cell types, including molecules expressed by the cell type. In some embodiments, markers are proteins. Such proteins can possess an epitope for antibodies or other binding molecules available in the art. A biomarker can consist of any molecule found in, on, or produced by (e.g., secreted) a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Biomarkers can be detected by any method available to one of skill in the art. Biomarkers can also be the absence of a morphological characteristic or absence of proteins, lipids etc. Biomarkers can be a combination of a panel of unique characteristics of the presence and/or absence of polypeptides and other morphological, functional, or structural characteristics.

As used herein, “treating” or “administering” are used interchangeably in the context of the placement of a cell composition, into a subject, by a method or route that results in at least partial localization of the compositions described herein at a desired site, such as the desired organ or a region thereof, such that a desired effect(s) is produced.

As used herein, the terms “disease” or “disorder” refers to a disease, syndrome, or disorder, partially or completely, directly or indirectly, caused by one or more abnormalities in the genome, physiology, or behavior, or health of a subject.

The terms “decrease,” “reduce,” “reduction,” or “inhibit” are all used herein to mean a decrease by a statistically significant amount. In some embodiments, “reduce,” “reduction,” “decrease,” or “inhibit” means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass complete inhibition or reduction as compared to a reference level. “Complete inhibition” is a 100% inhibition as compared to a reference level.

The terms “increased”, “increase”, “enhance”, or “activate” are all used herein to mean an increase by a statically significant amount. In some embodiments, the terms “increased,” “increase,” “enhance,” or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, a “subject” is a human or a non-human animal. Usually the non-human animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases including diseases and disorders involving inappropriate immunosuppression. A subject can be male, female, or intersex, meaning both male and female features are present.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at risk of developing that condition.

As used herein, a “reference level” refers to the level of a marker or parameter in a normal, otherwise unaffected cell population or tissue (e.g., a cell, tissue, or biological sample obtained from a healthy subject, or a biological sample obtained from the subject at a prior time point, e.g., cell, tissue, or a biological sample obtained from a patient prior to being diagnosed with a disease, or a biological sample that has not been contacted with a cell composition as disclosed herein). Alternatively, a reference level can also refer to the level of a given marker or parameter in a subject, organ, tissue, or cell, prior to administration of a treatment, e.g., with tissue stem cells provided herein or via administration of a transplant composition.

As used herein, an “appropriate control” refers to an untreated, otherwise identical cell, subject, organism, or population (e.g., a cell, tissue, or biological sample that was not contacted by an agent or composition described herein) relative to a cell, tissue, biological sample, or population contacted or treated with a given treatment. For example, an appropriate control can be a cell, tissue, organ or subject that has not been administered a tissue stem cell or differentiated progeny thereof as described herein.

The term “statistically significant” or “significantly” refers to statistical significance and generally means a difference greater than that defined by the 95% confidence interval.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Example 1

### A Technology for Rapid Inline Determination of Stem Cell-Specific Number During Tissue Stem Cell Biomanufacturing Processes

Overall objective: Develop a highly-efficient, and automated tissue dissociation and cell recovery technologies that maintain in-process and final CQAs (Critical Quality Attributes: viability, purity, potency, identity, sterility).

### Summary of the Method

The tissue stem cell counting technologies provided herein are crucial to successfully achieving this above objective. One important determinant of the functional variability of many human tissue starting materials for cell biomanufacturing is the variation in tissue stem cell fraction (SCF) and quality. Similarly, variation in tissue stem cell fraction and quality is a major determinant of subsequent biomanufacturing production success and the quality of produced, stored, and shipped tissue stem cell products. In addition, as will be outlined below, because of the computational basis of the developed tissue stem cell counting method, it is possible to design non-invasive approaches for its deployment to monitor important changes in stem cell fraction and quality during cell biomanufacturing.

Because of its focus on specific quantification of therapeutic human tissue stem cells, the technology provided herein is relevant to many elements of biomanufacturing. The methods provided herein were developed for specific application to stem cell expansion culture process development and execution. Because of the importance of specific quantification of tissue stem cells and their viability in many cell therapy products, the project's stem cell counting technology also addresses needs for the preservation, packaging, and storage process as well.

### Introduction

A major goal and need of regenerative medicine are technologies for the routine biomanufacture of therapeutic tissue stem cells. This long-standing need applies to both experimental stem cell treatments like mesenchymal stem cell (MSC) administration and approved stem cell treatments like hematopoietic stem cell (HSC) transplantation. In both cases, the development of industrial scale biomanufacturing processes has lagged greatly behind need and expectations. The number of companies claiming success in the large scale production of highly targeted MSCs for use in clinical trials development for regenerative medicine can be counted on one hand; and there are no examples of success in the expansion of HSCs on even a clinical scale, let alone industrial.

Though often misunderstood, confused, and overlooked, the reasons for the difficulty in achieving effective biomanufacturing of tissue stem cells are, in fact, well-established biological properties that, unfortunately, are uniquely characteristic of tissue stem cells.

The first reason is their essential property of undergoing asymmetric self-renewal divisions that maintain their own stem cell state, while simultaneously producing differentiating sister tissue cells. Based on simple mathematics, this high rate of asymmetric self-renewal makes it quite impossible to achieve the net accumulation of tissue stem cells in conventional cell culture systems. In fact, as a result of this defining property of tissue stem cells, conventional cell culture leads to their rapid dilution and loss among the non-stem, differentiating sister cells that they produce. Therefore, unless measures are taken to increase the rate at which tissue stem cells undertake the alternative symmetric self-renewal divisions, which produce two sister stem cells, effective tissue stem cell bio-manufacturing cannot be accomplished (FIG. 1). Not nearly enough research is supported to discover agents and/or conditions that can induce controlled, reversible symmetric self-renewal division by tissue stem cells. The discovery and development of agents with this property are crucial to the future success of tissue stem cell biomanufacturing.

The second reason, which is the purpose of this invention, has been so challenging to address for so long that it has become largely ignored and unrecognized. The now entrenched culture of ignoring this essential problem has engendered a current stem cell biomanufacturing industry that is blind to its own most important focus, tissue stem cells. For more than 60 years, the fields of stem cell biology and stem cell medicine have tried to make progress without a means to quantify the number of tissue stem cells. This deficiency existed in part because tissue stem cells are a low abundance tissue cell type—estimated to exist typically at tissue fractions less than 1 per thousand or ten thousand total tissue cells. This simple biological challenge might have been overcome if there were stem cell-specific molecular biomarkers that could be used with highly sensitive technologies like flow cytometry to detect and count tissue stem cells. But to date, no such stem cell-specific biomarkers have been discovered, despite many years, many dollars, and many careers spent attempting to identify even one.

Without a means to count tissue stem cells, all efforts to process engineering of their bio-manufacture are hamstrung. The few companies that claim to biomanufacture expanded populations of asymmetrically self-renewing tissue stem cells do so by suggesting that their cultures are predominantly stem cells in constitution. However, since they have not breached the mathematical consequences of asymmetric self-renewal by tissue stem cells, their cultures of serially passaged cells are predicted to have many fewer stem cells than their starter tissue cell preparations. Beyond this problem of quantifying stem cells in the final cell product, all attempts at optimization of tissue stem cell bio-manufacturing processes, based on increasing total cell proliferation, are necessarily ineffective and misleading. Total cell number is not a reliable indicator of tissue stem cells, which are present at very low fractions.

Asymmetrex has introduced the TORTOISE Test™, the first technology for specific and accurate counting of diverse human tissue stem cells, including MSCs, HSCs, and other tissue stem cell types of therapeutic interest like liver stem cells and corneal stem cells. Unlike previous attempts to count tissue stem cells, the new method does not rely on molecular biomarkers or assays in mice. Instead, a computational method is applied based on simulations of total cell count data collected from the serial passage of stem cell-containing cultures. The new method, called kinetic stem cell counting, leverages the mathematics of the unique asymmetric self-renewal of tissue stem cells to deduce their specific number based on how they limit the kinetics by which other cells are produced by them over time in culture. The TORTOISE Test™ also defines other previously inaccessible biological properties of tissue stem cells during culture, like their death rate and rates of symmetric divisions, which are required to accomplish their expansion in culture. The ability to monitor these unique properties specifically may also have important applications in the process engineering of effective tissue stem cell biomanufacturing practices.

Although the TORTOISE Test™ technology (i.e., PSCK-RIFS; 1-3) is now well-validated, it has a major operational shortcoming that has so far limited enthusiasm for its adoption as a new valuable tool for stem bio-manufacturing process engineering. The serial culture period needed to collect the necessary data for the computer simulation determination is significant, with a current minimum of about 3 weeks. This requirement is substantially less than the 3 to 4 months required for mouse assays used to estimate HSC number (See later); and for all other human tissue stem cell types, no reliable method of estimation is even available. Even so, the TORTOISE Test™'s shorter time requirement is still long enough to render it a cumbersome, retrospective analysis compared to the timescale of tissue stem cell production processes.

More recently, the inventors have devised method to extend the TORTOISE Test™ to being an effective, rapid, inline method for quantifying tissue stem cell number during biomanufacturing processes. In addition, because of the computational basis for the method, in the future it can be applied to design a non-invasive method for monitoring tissue stem cell fraction, inline, during cell biomanufacturing processes. Provided herein is a robust method for determination of the tissue stem cell fraction (SCF) of a test sample within 72 hours or less. The method can also be used to develop electronic tissue stem cell counters for the industry.

Despite the fact that no method existed to count human tissue stem cells specifically and accurately prior to the TORTOISE Test™ technology, confusion and misinformation in the regenerative medicine industry regarding the issue of stem cell quantification is persistent. There are several misnamed “stem cell biomarkers” that a significant fraction of industry participants think are able to quantify tissue stem cells. Examples of commonly mistaken and misrepresented biomarkers are CD34, CD133, and CD90. These molecular biomarkers are proteins expressed on respective types of tissue stem cells. The problem that is often not appreciated is that they are also expressed on the differentiated progeny cells produced by asymmetrically self-renewing tissue stem cells. These committed progenitor cells do not have the therapeutically sought durable, long term, tissue cell renewing properties of tissue stem cells, but they express the same so-called “stem cell biomarkers.” In freshly isolated tissue cell preparations, and even more so in cultured tissue cell preparations produced in cell biomanufacturing, committed progenitor cells can outnumber the tissue stem cells by 100-fold or more. Therefore, counts made with these, the currently best available molecular biomarkers, are not tissue stem cell counts, but instead committed progenitor cell counts.

There are two other main assays in wide usage that are often mistook for being able to determine the fraction of tissue stem cells. The colony forming unit (CFU) assay is prevalent in the cord blood banking industry for evaluating HSC fraction; and it is also used for estimating endothelial stem cell number. Both applications suffer from the well-known inability of the CFU assay to distinguish colonies formed by stem cells from colonies formed by committed progenitor cells. This lack of specificity is the long recognized cause of the CFU assay's failure to predict the HSC transplantation potency of umbilical cord blood units.

Unlike the CFU assay, the limiting-dilution SCID mouse repopulating cell (LDSRC) assay has superior specificity for detection of the activity of human HSCs, from adult donors or umbilical cord blood. Currently, gene therapy companies whose therapies target human HSCs use this assay to estimate the HSC fractions at different stages of their therapeutic development process. Despite its excellent specificity, the LDSRC assay has several significant drawbacks. Because of its requirement of a large number of mice to perform, it is very expensive. Requiring 12-16 weeks to complete, it lacks the timeliness needed for the process engineering of HSC biomanufacturing. Though it detects stem cells specifically, its quantification accuracy is limited by variability in the engraftment efficiency of tester mice. Finally, it is only applicable to HSCs and not other stem cell types in great demand for biomanufacturing, like mesenchymal stem cells.

In addition to the CFU assay and the LDSRC assay, there are other cell assays in the marketplace that quantify particular cellular metabolic activities that are also mistook to be tissue stem cell-specific. Therefore, none of these assays quantify tissue stem cells.

Unlike the TORTOISE Test™, none of the other currently available methods for attempting to estimate tissue stem cell fraction also provide determination of tissue stem cell-specific kinetics factors for viability, cell cycle time, and symmetric self-renewal rate. Each of these tissue stem cell properties could become important metrics for development, optimization, and monitoring throughout the entire tissue stem cell products supply chain.

Described herein is a method of counting tissue stem cells for a new software program for the determination of rapid tissue stem cell-specific counting algorithms from foundational TORTOISE Test™ tissue stem cell count analyses. The derived algorithms are under evaluation for their effectiveness in quantifying tissue stem cell production by industrial partners that isolate, biomanufacture, and supply tissue stem cell products. The tissue stem cell counts will be compared to the partners' existing production metrics (e.g., total viable cell number, “stem cell biomarkers,” cell differentiation markers) to establish how they relate and to investigate whether TORTOISE Test™ tissue stem cell-specific counts are earlier predictors of later cell production outcomes.

### Technical Scope and Objectives

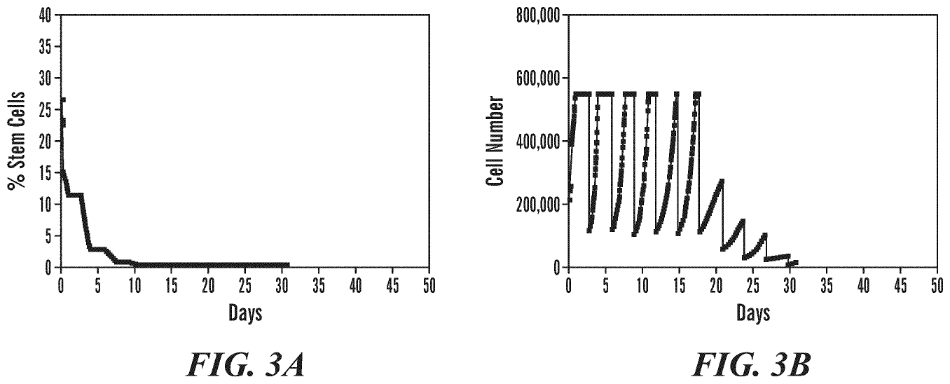

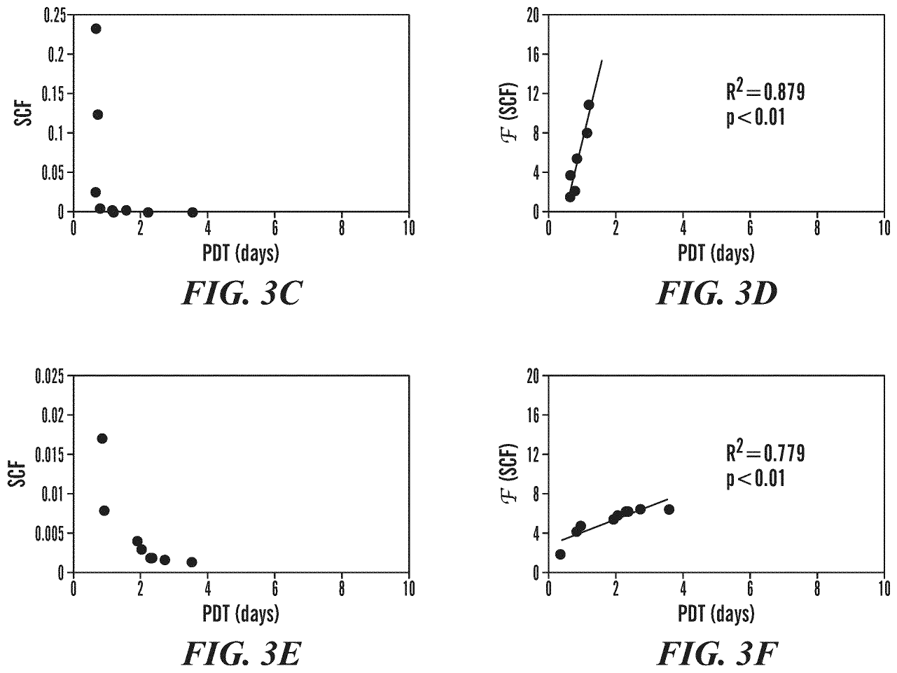

The patented TORTOISE Test™ technology for counting tissue stem cells specifically and accurately was introduced to the biomanufacturing industry by Asymmetrex in 2016. Since then, experience has increased with its application for counting diverse tissue stem cell types in diverse tissue cell preparations, including adherent and non-adherent cells. Recently, the data output from foundational TORTOISE Test™ studies was recognized to contain information that relates simple 72-hour cell culture population doubling time (PDT) data to corresponding stem cell fraction (SCF) data. Mathematical interrogation of these data comparisons revealed underlying mathematical functions that defined a PDT:SCF relationship with a high degree of statistical confidence (See FIG. 3D and FIG. 3F).

Evaluation of the new invention has had three main objectives to date:

Objective 1—Automate the stem cell counting algorithm discovery process by writing a new software program for rapid data analysis, algorithm determination, and statistical evaluation.

Objective 2—Use commercial cell sources to define the stem cell counting algorithms for four different types of tissue stem cells with high interest for stem cell medicine (and supplied by industry partners): mobilized CD34+ HSCs, cord blood-derived CD34+ HSCs, adipose tissue-derived MSCs, and umbilical cord-derived MSCs. Use at least two different commercial sources of each tissue stem cell type to evaluate the consistency of stem cell type-specific algorithms.

Objective 3—Define tissue stem cell counting algorithms using cell production samples from partner biomanufacturing companies. Compare to congruent algorithms determined with commercial cell sources for Objective 2. Evaluate stem cell fraction (SCF) determinations at early stages of partners' production processes for whether they are predictive of later production outcomes.

### Technical Approach

As detailed above, the TORTOISE Test™ platform was the first technology for specific and accurate counting of human perinatal and postnatal tissue stem cells. More recently, the TORTOISE Test™ technology was used to invent the PDT:SCF stem cell counting algorithms that now afford significantly more rapid tissue stem cell counting that is more suitable for cell biomanufacturing process engineering and monitoring.

The first step in reducing to practice the invention was writing new software for automating the algorithm discovery and development process. With the new software in hand, PDT:SCF stem cell counting algorithms are in development for four different types of stem cell preparations currently of great therapeutic interest: adipose tissue-derived MSCs, umbilical cord-derived MSCs, mobilized CD34⁺ HSCs, and cord blood CD34⁺ HSCs. Two different commercial sources of each type of stem cell preparation are under evaluation. These analyses will allow a determination of common aspects of stem cell-specific counting algorithms for different preparations of the same stem cell types.

With the experience developed with the commercial stem cell products, effort will be turned to evaluation of production samples from collaborating stem cell biomanufacturing and supply industry partners. The analyzed samples will cover early, middle, and late stages of production. By comparing SCF data to partners' current quality metrics (e.g., total cell viability, phenotypic biomarkers, differentiation efficiency), the diagnostic value of SCF and its ability to provide a better predictor of production run outcome will be assessed.

The new PDT:SCF algorithms have the potential to also provide a non-invasive method of monitoring tissue SCF during biomanufacturing processes. This is because the total cell number can be monitored by spectral means related to culture turbidity by either light refraction or light absorption. As long as the measured factor is directly related to changes in total cell number, it can be used to calculate culture PDT. By having a glass window into cell culture reactors or a glass loop that allows monitoring of the total cell number spectrally, it will be possible to apply validated stem cell counting algorithms for real-time, inline estimates of the SCF during cell biomanufacturing. An indwelling current resistance sensor, as is used to monitor cells in some counting instruments, might also suffice for this purpose.

Relevant Prior Work Toward this Specific Effort, Current Status, and Results

The following section describes how the TORTOISE Test™ technology, in its initial formulation, can specifically and accurately count diverse human tissue stem cells, including the stem types proposed for evaluation with the invention. Thereafter, the mathematical process utilizing TORTOISE Test™ data outputs are described for defining the tissue stem cell counting algorithms that can be applied for rapid determination of tissue stem cell fraction (SCF). Because of the importance of SCF in determining the long-term proliferation of any human tissue cells in culture, ready access to this factor throughout cell biomanufacturing processes should enable many important advantages and advances for the industry.

### Introduction to the TORTOISE Test™ Technology

To perform the TORTOISE Test™, a tissue cell preparation of interest is first counted for total viable cells; and then, based on the cell count, a defined number of live cells is used to initiate replicate serial cultures, typically in triplicate. At an experimenter-defined interval, cultures are counted—including live cell and dead cell counts—and a constant fraction of cells or constant number of cells is transferred to a new culture dish of the same type. These counted passages are continued until no increase in cell number occurs, which is an invariant property of all natural, normal human tissue cell cultures. The current TORTOISE Test™ Probabilistic Stem Cell Kinetics (PSCK) software is designed to be versatile for accommodating different formats for the passage time interval and/or the number of cells transferred. The total cell count data are transformed into plots of cumulative population doublings (CPD); and the viable cell fraction is used to estimate the dead cell fractions for transient cells (RDT in FIG. 1) and terminal cells (RDTM in FIG. 1), which are similarly abundant.

FIG. 1 depicts the various inputs that are used by the PSCK software to compute CPD curve simulations for the period defined by the experimental CPD data. Using the cellular model illustrated in FIG. 1, the software inputs measured values (“m” superscripted in FIG. 1) and discovered factors (“d” superscripted in FIG. 1) obtained from the TORTOISE Test™ Random Input Factor Searching (RIFS) software. The RIFS software randomly generates values for the unknown factors required to simulate the serial population doublings of cells based on the illustrated cellular model for the production of transient amplifying cells and differentiated cells by the asymmetric self-renewal of tissue stem cells. In the programming, RIFS-selected random factors are input into the PSCK simulation software. The PSCK simulation software uses these discovered factors along with the measured factors to calculate CPD curve simulations. The calculated simulations are then compared by root mean squared error analyses to identify sets of discovered factors that yield CPD simulations that quantitatively best describe the experimental CPD data.

### Validations of the TORTOISE Test™ Technology

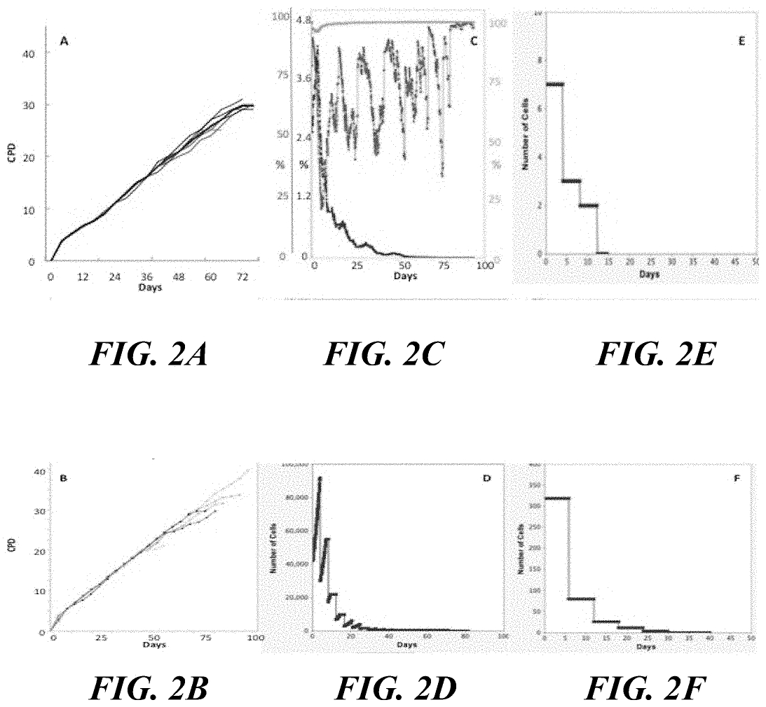

As shown in FIG. 2, the first validation of the TORTOISE Test™ was the ability of the PSCK-RIFS software to generate statistically confident simulations of experimental CPD data (e.g., compare FIG. 2B to 2A). Among the unknown factors that the simulations discover is FR_NS, the initial stem cell fraction (SCF; FIG. 1).

The TORTOISE Test™ software can do more than just count stem cells. It can also compute estimates of all the factors in FIG. 1 and predict changes in their value during serial passage. This new access to previously unknowable properties of tissue stem cells will have many innovative applications in cell biomanufacturing process engineering.

Multiplying FR_NS times the initial total cell input gives the number of stem cells in the evaluated tissue cell sample. Once unknown factors like FR_NS (i.e., SCF) are discovered by PSCK-RIFS analysis, they can be used with the PSCK software to output component cell kinetics features that were previously inaccessible. For example, changes in the relative fractions of all three modeled cell types during serial passaging can be independently examined (FIG. 2C). A particularly powerful computation is the stem cell number (and corresponding SCF) during serial passage. Stem cells from all tissues examined so far show a characteristic decline in number and fraction that is predicted based on continuation of their in vivo asymmetric self-renewal kinetics in culture (FIG. 2D-F). This analysis can identify stem cell types, like expanded human liver stem cells, that have higher than usual rates of symmetric self-renewal in culture (FIG. 2D). The TORTOISE Test™ experience to date includes analyses of 4 different types of stem cells (i.e., hepatic, HSCs, corneal, and mesenchymal) from 9 different human tissues, including liver, lung, bone marrow, umbilical cord, umbilical cord blood, amniotic membrane, cornea, dental pulp, and adipose tissue. Based on these analyses, the TORTOISE Test™ has been shown to afford a 1000-fold dynamic range for tissue stem cell fraction determinations.

Because of these fundamental cell kinetics relationships, the proliferation rate of any normal human tissue culture is related to the number of stem cells it contains, its stem cell fraction (SCF; FR_NS in FIG. 1). A universal measure of cell culture proliferation rate is the population doubling time, PDT. PDT is the time required for a cell culture to double in total cell number. Typical human tissue cell culture PDTs range from 12 to 60 hours. Based on these ideas, it can be possible to deduce the number of tissue stem cells in any culture from the culture's PDT, which can be determined by simple cell counting in 24 hours or less. However, such a strategy could not have been envisioned by those practiced in stem cell culture before this invention, because it was not imaginable that the SCF could be determined or known throughout an entire period of serial culture.

The diagram in FIG. 1 makes it clear why any analysis to deduce the SCF from the culture PDT would be considered a formidable undertaking. One needs to know all the many other cell kinetics factors involved to develop the incisive calculation that would yield the SCF from a culture's PDT. Although some of the cell kinetics factors in FIG. 1 could be measured directly, many were inaccessible with extant technology. The TORTOISE Test™ technology deploys computational simulation strategy to discover these unknown factors for any human tissue cell culture.

The unique output of the TORTOISE Test™, the SCF over time throughout serial culturing, makes it possible to define mathematical algorithms that yield the SCF from a culture's simple PDT. This invention and its development are outlined by the data shown in FIGS. 3A-3F. The current TORTOISE Test™ software gives the two relevant data outputs. The first is the unique SCF throughout the foundational serial culture analysis (FIG. 3A). FIG. 3B shows an example of the second output, the corresponding data for the total culture cell number versus days of serial culture. The vertical declines in the data are simulations of culture dilutions to start next cultures in the series. The curvilinear inclines from dilution low points reflect the simulated multiplication of the diluted cells. The PDT between any two time points of culture can be calculated by:

$$PDT = \frac{{t}_{2} - {t}_{1}}{ln ( \frac{{N}_{2}}{{N}_{1}} )} ln 2 , \qquad ( Formula A )$$

where t₁=initial starting time, t₂=final time, N₁=the initial cell number, and N₂=the final cell number.

Computation of the PDT determined from time=0 of serial culture to the first maximum and for each incline in cell number from each successive dilution low point to the next maximum in FIG. 3B can be performed and related to the corresponding SCF values at time=0 and at each culture dilution low point. These data are shown in FIGS. 3C and 3E for two different types of tissue stem cells. Both show the predicted characteristic increasing culture PDT, as the SCF decreases. This relationship is an orthogonal validation of the key principle upon with the TORTOISE Test™ is based, namely that tissue stem cells are rate-determining for culture proliferative rate. As shown in FIGS. 3D and 3F, based on these analyses, mathematical functions can be readily defined that linearize the PDT vs. SCF data into statistically significant descriptions of the data. Similar analyses based on PDT values, calculated with the experimental cell count data for the simulations, yield similar results. These transforming “PDT:SCF tissue stem cell counting algorithms” should make it possible to calculate confident estimates of the SCF of the same types of stem cells, when grown under the same culture conditions, from the PDT determined after 72 hours or less of cell culture.

### Example 2

### A Computational Simulation Technology for Specific Counting of Perinatal and Postnatal Human Tissue Stem Cells for Transplantation Medicine

Although tissue stem cells are essential for the maintenance, renewal, and repair of vertebrate organs and tissues, previously, the simple act of counting them has not been possible. For more than a half-century, progress in tissue stem cell research and medicine has been undermined by the lack of a means to determine tissue stem cell number. In particular, a major unmet need for stem cell transplantation medicine has been a way to quantify the specific dosage of tissue stem cell treatments. The counting problem persists because no biomarkers are known that identify tissue stem cells specifically, without also counting their more abundant committed progenitor progeny cells. Here, a description is provided of the integration of principles of tissue stem cell asymmetric self-renewal kinetics with computational simulation to achieve specific and accurate counting of therapeutic tissue stem cells. The asymmetric self-renewal kinetics of tissue stem cells is rate-limiting for the rate and extent of proliferation of primary tissue cell cultures. Based on this essential relationship, this description shows that simple total cell count data from primary cell cultures, passaged until achieving terminal proliferation arrest, are determined by tissue stem cell kinetics factors, including viability, cell cycle time, self-renewal rate, and number. Probabilistic Stem Cell Kinetics (PSCK) model and Random Input Factor Searching (RIFS) software are described that can be combined to discover these previously inaccessible factors. The method is validated by several orthogonal strategies, including comparisons to the currently best available method for estimating a tissue stem cell fraction, independent tests for asymmetrically self-renewing cells, tissue stem cell fractionation, and treatments with tissue stem cell-active agents. A major advance of the method is the discovery of simple algorithms that allow rapid convenient computation of the specific tissue stem cell fraction (SCF) of complex human tissue cell preparations from simple population doubling time (PDT) data.

Though often overlooked and poorly understood, a stem cell counting problem for perinatal and postnatal vertebrate tissues, including those of humans, has persisted for more than a half-century [1-3]. Two biological properties of adult tissue stem cells (i.e., perinatal and postnatal tissue stem cells) are mainly responsible for this situation. First, because stem cells exist in low fractions in tissues, they also have low fractions in isolated tissue cell preparations. This tissue property is often unappreciated because of the common misconception and misrepresentation that stem cell-containing preparations are homogenous for stem cells. In fact, the most enriched populations of adult tissue stem cells rarely exceed fractions greater than a few percent. Second, commonly misnamed “stem cell biomarkers” used for tissue stem cell research are also expressed by much more abundant committed progenitor cells [1-3]. As the progeny of asymmetrically self-renewing stem cell divisions, based on their molecular expression properties defined to date, committed progenitors have been indistinguishable from their stem cell parents. Since committed progenitor cells outnumber stem cells by orders of magnitude both in vivo and in isolated tissue cell preparations, present day tissue “stem cell biomarkers” actually give only a committed progenitor cell count, and no stem cell count at all.

Despite the long-standing unmet need for methods for specific and accurate counting of perinatal and postnatal tissue stem cells, there is significant misinformation on this issue in stem cell research and stem cell medicine. Though not all, many scientists, physicians, and technologists operate with the misperception that tissue stem cell counting is possible and that the stem cell-specific dosage of treatments is known. The basis for this confusion is two-fold, being related to the language of stem cell science and medicine and to the limitations of the available methods that attempt to quantify tissue stem cells. First, the language of tissue stem cell quantification is often not sufficiently exact. Recent controversy over the constituents of mesenchymal stem cell (“MSC”)-containing tissue cell preparations and the naming of the preparations is illustrative of this problem [4]. MSC preparations are not homogeneous collections of tissue stem cells. Like all isolated, enriched, or expanded tissue cell preparations, they are heterogeneous. They contain mesenchymal stem cells, lineage-related mesenchymal committed progenitor cells, and both lineage-related and non-lineage mature cells. The mesenchymal stem cell fraction is a small proportion of total cells. Low stem cell fraction is characteristic of all types of stem cell-containing tissue cell preparations, including enriched populations like CD34+-selected cell fractions containing hematopoietic stem cells (HSCs). When correct exact language is used, tissue stem cell-containing preparations are called “stem/progenitor cells.” However, often used less exact language, like “stem cells” per se, is routinely applied inappropriately and fosters the mistaken idea that designated stem cell preparations are homogenous for stem cells, when in fact they never are. This error in characterization leads to the misperception that simple counts of the total cell number are equivalent to a stem cell counts and stem cell dose determination, though they are not. Some investigators working with “MSCs” have suggested renaming the preparations to acknowledge the uncertainty of cellular constituents [4]. However, their solution, though more exact, obscures the significance of the crucial cellular constituent, the stem cell fraction (SCF). A means to provide the stem cell-specific fraction of MSC preparations is a better solution all around.

A second cause of significant misinformation and confusion about the state of tissue stem cell quantification methods is misunderstanding of the capabilities of existing methods for estimating tissue stem cell number. A very common belief is that tissue stem cells can be counted by flow cytometry. However, although antibodies exist that allow tissue stem cells to be enriched by either fluorescent activated cell sorting (FACS) or immunoselection, the same antibodies cannot be used to count tissue stem cells by flow cytometry. As noted earlier, all known molecular biomarkers expressed by tissue stem cells are also expressed by their committed progenitor cell progeny [1-3]. Since, committed progenitor cells outnumber stem cells significantly both in vivo and in isolated stem cell-enriched cell populations, but appear the same, they prevent quantification of the smaller fraction of tissue stem cells, preventing the latter's quantification by flow cytometry.

Despite the fact that previously no method existed to count human tissue stem cells specifically and accurately, confusion and misinformation in tissue stem cell science and medicine about the issue of tissue stem cell quantification has been persistent. There are several misnamed “stem cell biomarkers” that are commonly mistaken for being able to quantify tissue stem cells. Examples of misrepresented biomarkers are CD34, CD133, and CD90 [1-3]. These molecular biomarkers are proteins expressed on respective types of tissue stem cells. As noted before, their often-unappreciated shortcoming is that they are also expressed on the differentiated progeny cells produced by asymmetrically self-renewing tissue stem cells. These committed progenitor cells do not have the therapeutically sought durable, long term, tissue cell renewing properties of tissue stem cells, but they express the same so-called “stem cell biomarkers.” In freshly isolated tissue cell preparations, and even more so in cultured tissue cell preparations produced in cell biomanufacturing [5], committed progenitor cells can outnumber the tissue stem cells by 100-fold or more. Therefore, counts made with these, the currently best available molecular biomarkers, are not tissue stem cell counts, but instead committed progenitor cell counts [1].

There are two other main assays in wide usage that are often mistaken for being able to determine the fraction of tissue stem cells. The colony forming unit (CFU) assay is prevalent in the cord blood banking industry for evaluating the hematopoietic stem/progenitor cell fraction; and it is also used for estimating endothelial stem/progenitor cell number. Both applications suffer from the well-known inability of the CFU assay to distinguish colonies formed by stem cells from colonies formed by early committed progenitor cells. This lack of specificity is the long recognized cause of the CFU assay's failure to predict the HSC transplantation potency of umbilical cord blood units [6].

Unlike the CFU assay, the limiting-dilution SCID mouse repopulating cell (LDSRC) assay has superior specificity for detection of the activity of human HSCs, from adult donors or umbilical cord blood. Currently, gene therapy companies whose therapies target human HSCs use this assay to estimate the HSC fractions at different stages of their therapeutic development process. Despite its excellent specificity, the LDSRC assay has several significant drawbacks. Because of its requirement of a large number of mice to perform, it is very expensive. Requiring 12-16 weeks to complete, it lacks the timeliness needed for the process engineering of HSC biomanufacturing and dosage determination for stem cell treatments. Though it detects stem cells specifically, its quantification accuracy is limited by variability in the engraftment efficiency of the tester mice. Finally, it is only applicable to HSCs and not stem cells from other tissues [7].

In addition to the CFU assay and the LDSRC assay, there are other cell assays in the commercial tissue stem cell marketplace that quantify particular cellular metabolic activities that are also mistaken to be tissue stem cell-specific [8]. Therefore, despite their representation, none of these assays quantify tissue stem cells. In addition, unlike the new tissue stem cell counting invention described herein, none of the other currently available methods for attempting to estimate tissue stem cell fraction also provide determination of tissue stem cell-specific kinetics factors for viability, cell cycle time, and symmetric self-renewal rate. Each of these tissue stem cell-specific properties could become important new metrics and tools for accelerating progress in tissue stem cell science and medicine.

There is one property of adult tissue stem cells that does distinguish them from committed progenitor cells. This property is their asymmetric self-renewal kinetics [9, 10]. Asymmetric self-renewal is the process by which adult tissue stem cells continuously divide with simultaneous renewal of themselves and production of lineage-committed progenitor cells. Herein, the design and validation of a first method is described for specific and accurate counting of stem cells from diverse human tissues based on the concept that asymmetric self-renewal by adult tissue stem cells defines them exclusively and is rate-determining for the overall proliferation of primary human cell preparations. The new counting technology addresses many previously unmet needs in tissue stem cell research, tissue stem cell transplantation medicine, drug development, and toxicology. It can determine, for the first time, the number of tissue stem cells in experimental cultures, the dose and quality of therapeutic stem cell preparations, and positive or negative effects of agents on tissue stem cell function specifically.

To determine the stem cell-specific fraction of human tissue cell preparations, the presented approach integrates computer simulation with a bioengineered model of the cell kinetics of total cell production that depends on the asymmetric self-renewal of tissue stem cells present in cultures. This bioengineered computational approach yields important, previously inaccessible, tissue stem cell kinetics factors, in addition to the stem cell-specific fraction. The new method quantitatively differentiates stem cells from committed progenitor cells for the first time.

To validate the new tissue stem cell counting method, several different orthogonal validation bases are presented. Since there is no existing method for counting tissue stem cells, validation was based on both comparisons to the best available estimates of a tissue stem cell fraction and predicted changes in stem cell fraction after experimental manipulation. Published estimates of human HSC fraction, determined by LDSRC assays, were used as best available estimates of a tissue stem cell fraction for comparison. Comparisons were also made to published determinations of the fraction of asymmetrically self-renewing cells, because this is the primary factor quantified by the counting method. In addition, validation was obtained by confirming changes in tissue stem cell fraction determinations in response to treatment of primary cell cultures with agents either known or suspected to cause changes in tissue stem cell self-renewal or viability. In the case of stem cell-toxic agents, these validation analyses also provided the first demonstration that a chemotherapy agent had specific toxicity against tissue stem cells compared to committed progenitor cells.

As a computational approach, the new method also provides, for the first time, the opportunity to relate changes in tissue stem cell fraction directly to changes in the population doubling time of primary human cell cultures. Such unique analyses reveal mathematical algorithms that now allow the rapid and convenient determinations of the stem cell fraction of complex human tissue cell cultures requiring only simple, readily available cell culture population doubling time (PDT) data. The advances described here close the chapter on adult tissue stem cells as elusive, unquantifiable elements of vertebrate tissues; and open a new chapter of accelerated progress in stem cell research, stem cell transplantation medicine, and drug discovery.

### Materials and Methods

### Cells

Strain WI-38 pre-senescent human lung cells were purchased from the American Type Culture Collection (Manassas, Va., USA; ATCC #CCL-75; passage 15). Expanded human liver stem cell strain SACK-XS 12(3) was purchased from Kerafast (Boston, Mass., USA; #EJS002). CD34+-selected normal human bone marrow cells were purchased from the ATCC (#PCS-800-012). CD34+-selected human umbilical cord blood cells were purchased from STEMCELL Technologies, Inc. (Cat#70008; Cambridge, Mass.).

Strain WI-38 cells were later recognized as being derived from the tissues of an electively aborted human fetus [11]. As a result of this awareness, strain WI-38 cells will not be used in future studies by the inventor. Discontinuation of the use of these cells is also recommended for future studies by others.

### Cell Culture

Serial cultures of WI-38 cells were performed in Dulbecco's Minimal Essential Medium (DMEM) supplemented with 10% dialyzed fetal bovine serum (dFBS). Starting with an input of 65,000 viable (i.e., trypan blue-negative) cells in 5 mLs medium in wells of 6-well culture plates, six replicate cultures were serially passaged every 96±4 hours by transferring ⅓ of the total cells to a new culture well. Triplicate total and viable cell counts were performed for each serial culture at each passage. Passaging was continued until two successive cell counts showed no significant increase in total cell number above the input cell number for the culture.

Expanded human liver stem cell strain SACK-XS 12(3) cells were cultured in the supplier's recommended culture medium without inclusion of xanthosine. CD34+-selected normal human bone marrow cells were cultured in StemSpan™ SFEM II medium (STEMCELL Technologies, Inc., Cambridge, Mass., USA; #09655) supplemented with StemSpan™ CD34+ Expansion Supplement (STEMCELL Technologies, Inc., #02691). SACK-XS 12(3) serial cultures were initiated in 6-well plates with 325,000 viable cells. CD34+ cell serial cultures were initiated as 24-well suspension cultures with 65,000 viable cells. For both cell types, ⅓ of total cells were transferred to a new culture well every 96±4 hour interval. Tested agents were supplemented at all times during the culture period. Passaging was continued until successive total and viable cell counts showed no significant increase in total cell number above the transferred cell number.

For CD34+-selected human umbilical cord blood cells, serial 24-well suspension cultures were initiated with 177,000 total cells in StemSpan™ SFEM II culture medium (Stemcell Technologies, Cat#09655) supplemented with StemSpan™ CD34+ Expansion Supplement (Stemcell Technologies, Cat#02691) and 1% penicillin/streptomycin. Triplicate suspension cultures were passaged with 1:5 splits every 72±1.4 hours until two successive cell counts showed no significant increase in total cell number above the transferred cell number.

All other culture cell count data were obtained from the referenced published reports.

### Correction Analysis for Initial Stem Cell Fraction

Initial PSCK-RIFS analyses revealed that some human tissue stem cells have a measurable, significant rate of symmetric self-renewal during cell culture. Because the resolution of the PSCK-RIFS determination of the initial stem cell fraction of input cells was at first limited by the size of the initial passage interval (3-4 days), cultures that had a significant degree of symmetric self-renewal yielded an initial stem cell number (NSo below) that was higher than the actual value before culture. Based on the determined stem cell symmetric self-renewal frequency (RS), in the early studies presented herein a correction was applied to account for this effect. The correction is determined by the following Equation 1:

NSo corrected=NS1st interval/(RSe^{kt}+RA−RDS) (Equation 1)

In Equation 1, NS1st interval=NS peak value determined from the initial interval of PSCK; t=the time to the first interval NS peak in the respective simulation; k=ln 2/GTS, the mean generation time for symmetrically self-renewing stem cells; RS, RA, and RDS are mean cell kinetics factors determined by the originating PSCK-RIFS analysis (See EXAMPLE 4). The mean of 10 independent PSCK NS simulations was used for correction. Equation 1 was applied to determine the initial stem cell fraction whenever a significant RS value was found.

Subsequently, the PSCK software programming has been revised, so that FR_NS corresponds to the SCF of analyzed cell preparations at time=0, prior to the start of cell culturing.

### Statistical Analyses

The simulation confidence level was estimated from the root mean squared error of the comparison of simulated cumulative population doubling (CPD) data to experimental CPD data. Student's t-test (2-tailed) was used to calculate p-values for confidence for cell kinetics factors determined by the PSCK-RIFS software (See also EXAMPLE 4) with respect to 0.0 and their 95% confidence intervals. Student's t-test (2-tailed) was also used to calculate p-values for confidence for differences in the value of cell kinetics factors derived under different culture conditions.

### Results

### The Cellular Basis for a Probabilistic Stem Cell Kinetics (PSCK) Model for the Production of Normal Tissue Cells in Culture

The unique asymmetric self-renewal of adult tissue stem cells was considered as a basis for quantifying them. In vivo, tissue stem cells are postulated to divide with asymmetric self-renewal kinetics [10, 12, 13]. By this cell kinetics program, stem cells can divide to produce cells committed to differentiation while maintaining their own number and stem cell properties. Illustrated in FIG. 1, this stem cell kinetics model includes both deterministic and probabilistic features. An essential probabilistic feature is the likelihood that the stem cell divides symmetrically versus asymmetrically. Symmetric divisions produce stem cell duplication, which increases the stem cell tissue fraction [14]; whereas asymmetric divisions maintain and renew tissue cells while preserving stem cell number [10, 12, 13].

Asymmetric cell kinetics by tissue stem cells can explain the characteristic eventual growth cessation of pre-crisis and pre-senescent serial cell cultures [15, 16]. Another hypothesis proposed to explain these respective rodent and human cell division arrests is telomere erosion. However, one feature of crises and senescence is not accounted for by telomere erosion. Both are cell density-dependent processes [18, 19]. Serial passage at higher cell densities delays the cell division arrest of cultures. This phenomenon can be explained by asymmetric self-renewal kinetics, which are highly density-dependent, with symmetric self-renewal being more probable at higher cell densities [14, 15].

In the absence of mutations that disrupt asymmetric self-renewal kinetics, cell cultures derived from diverse mammalian tissues undergo total division cessation after serial passage at sufficiently low cell dilutions. Called crisis for rodent cell cultures, the division cessation is often followed by a later recovery of cell division, an event called immortalization [20]. In contrast, the senescence arrest of all human tissue cultures is terminal, with one described exception. Fibroblasts from Li-Fraumeni Syndrome patients, who have a heterozygous p53 gene mutation, do undergo immortalization, but with loss or mutation of the wild-type p53 allele [21]. In a related fashion, immortalized rodent cell lines invariably have acquired p53 gene mutations [22] and lost the ability to undergo asymmetric self-renewal kinetics [15, 23]. Moreover, restoration of wild-type p53 function restores asymmetric self-renewal kinetics [15, 23].

Based on earlier observations of p53 effects on asymmetric stem cell kinetics, the total cell output of serial mammalian tissue cell cultures was postulated to be related to the fraction and self-renewal pattern of their tissue stem cells [15]. When the frequency of asymmetric self-renewal divisions by stem cells exceeds the frequency of symmetric self-renewal divisions, serial passaging will cause the loss of tissue stem cells as a result of their dilution among their differentiated progeny cells [5, 14-16]. Therefore, “crisis” or “senescence” as described are primarily states of cell division arrest due to the absence of stem cells. The number of passages required to achieve cell culture division arrest is related to factors that describe the cell kinetics of tissue stem cells; their dividing progeny cells; terminal non-dividing mature lineage cells produced; the number of cells transferred at each passage; and the length of the culture period between passages (See EXAMPLE 4).

### Integration of Cell Culture Data and Computer Simulation to Develop a Method for Counting Adult Tissue Stem Cells

Based on the cellular model in FIG. 1, software was developed that simulates cumulative population doubling (CPD) data computed for serially passaged cultures based on using inputs for both measured culture factors (i.e., starting viable cell number, passage interval length, number of cells passaged, general cell viability, maximum cell number achievable) and unknown cell kinetics factors like stem cell fraction (See EXAMPLE 4). The software program also includes probabilistic variance factors for all input factors. Some variances were known from experiment (e.g., the coefficient of variation [COV] for transient amplifying cell generation time (22%) was determined by time-lapse microscopy); but most were assumed to fall within the typical range for cell culture and cell counting experimental errors (COV=5-10%).

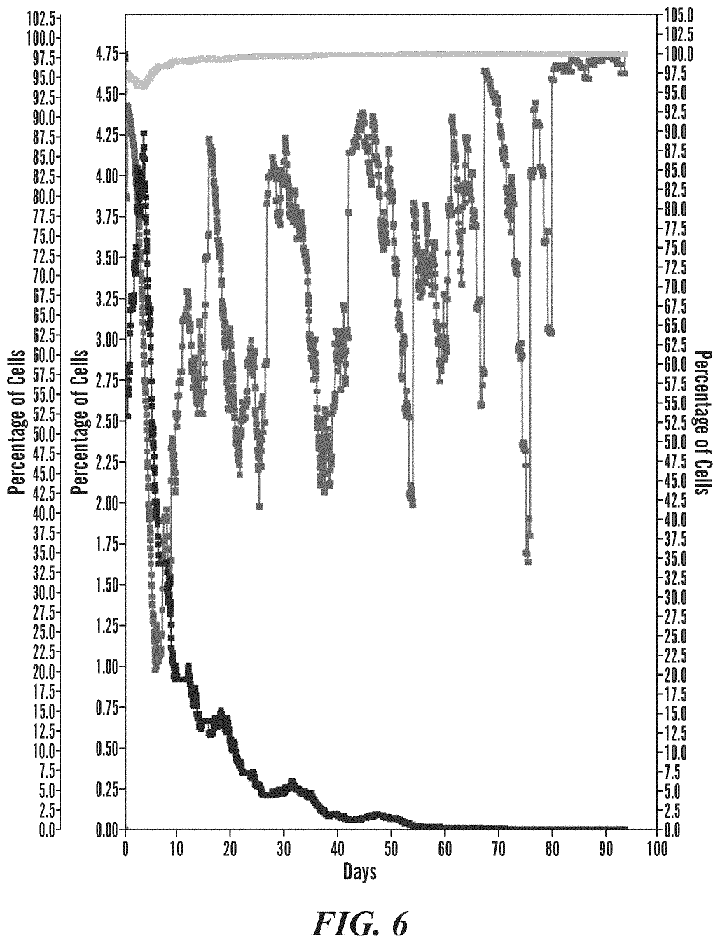

Given these initial input factors, the Probabilistic Stem Cell Kinetics (PSCK) software computes families of CPD curves within specified, biologically relevant probabilistic ranges. From these simulations (FIG. 2B), the PSCK software can delineate quantitative changes in the specific cell kinetics properties of the different cell types (See FIG. 1) over the course of serial passaging, including in particular those of tissue stem cells. FIG. 2C (also FIG. 6) provides an example of this powerful computational feature of the method. Based on the differences in their cell kinetics features, all of the different cell types evolving in complex primary cell cultures can be delineated quantitatively. The computations in FIG. 2C, derived from the simulation in FIG. 2B, show the independent changes in the fractions of tissue stem cells, transiently amplifying progenitor cells, and terminally-arrested differentiated cells during serial culture. Further cell kinetics delineations are also possible, like asymmetrically self-renewing stem cells versus symmetrically self-renewing stem cells (data not shown).

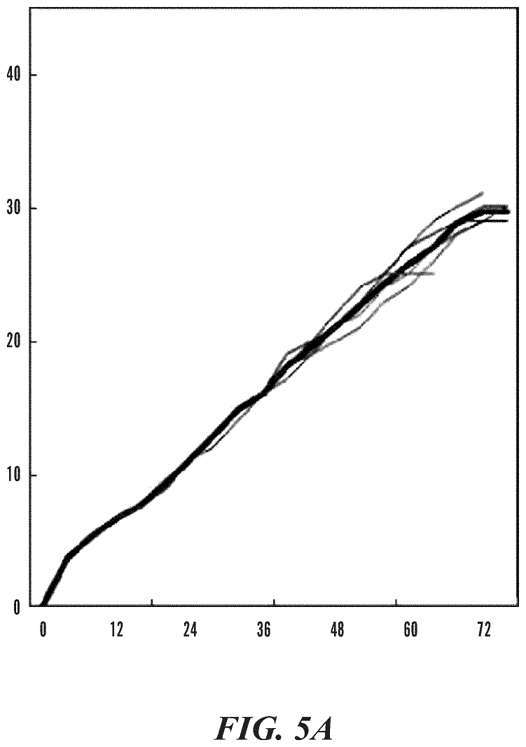

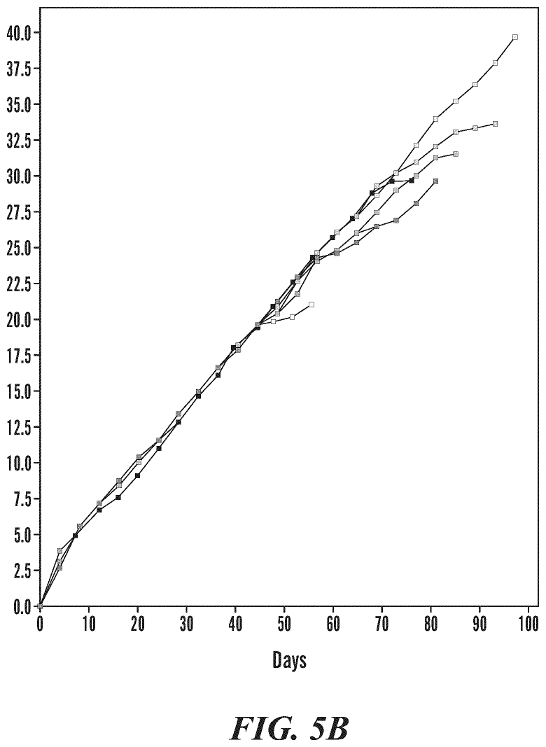

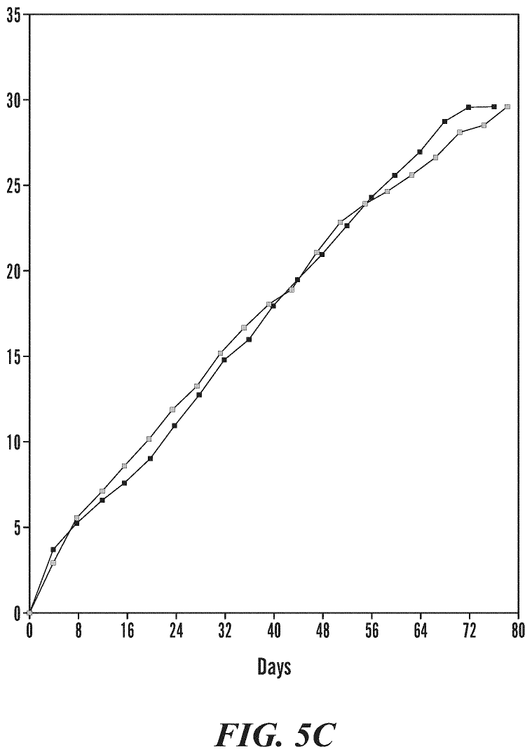

To discover the unknown input factors required for PSCK simulations, a second integrated software program was developed for Random Input Factor Searching (RIFS; See EXAMPLE 4). The RIFS software simultaneously evaluates multiple, randomly-selected values for unknown input factors for the quality of PSCK simulations they yield compared to both the magnitude and the variance of replicate (n=3 to 6) experimental CPD data. As shown in FIGS. 2A-2F (Compare FIG. 2A to FIG. 2B) and amplified in FIGS. 5A-5C, the RIFS program is able to discover input factor sets that produce CPD simulations that match experimental CPD with high statistical confidence. Importantly, the PSCK simulations not only achieve similar magnitude and variance, but they also show the cessation of cell division that is characteristic of primary mammalian cell cultures.

### Validations of the PSCK-RIFS Method for Counting Adult Tissue Stem Cells

A first and essential validation of the new counting technology was its ability to simulate experimental CPD data with a high degree of statistical confidence (e.g., FIGS. 2B, 2C, and 5C). Published serial cell culture data was also used to extend the range of the different types of tissue stem cells evaluated. Although the published CPD data were generally from single serial cultures, the PSCK-RIFS analyses were sufficiently robust to yield results of high statistical confidence (See Table 1).

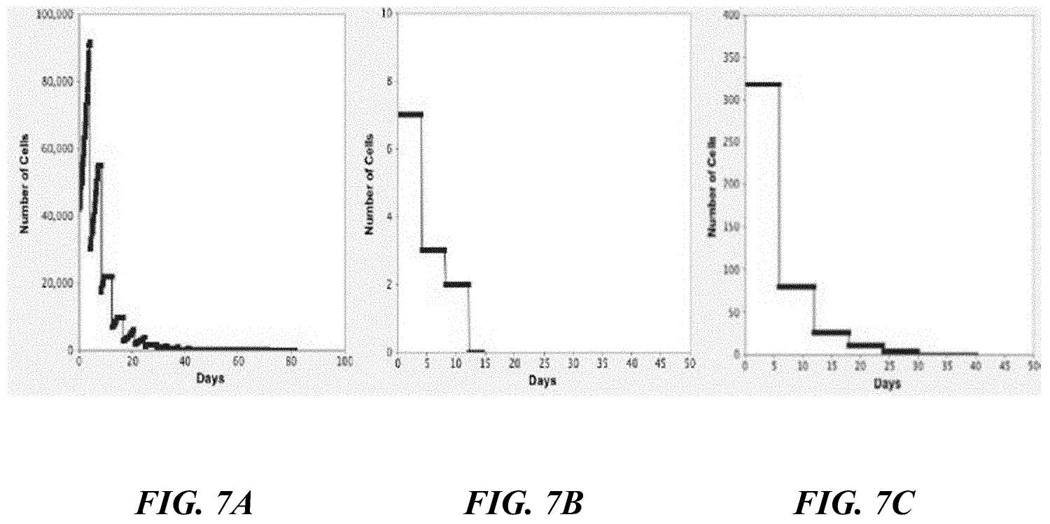

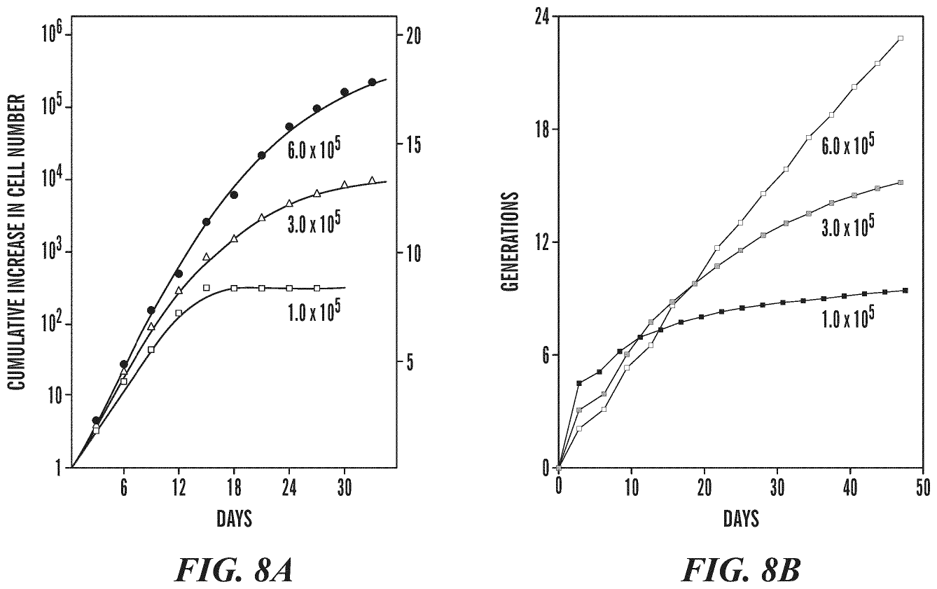

Several different orthogonal analyses were used to validate the method further. The stem cells in all tissues examined to date were determined to decline in fraction with serial passaging (FIGS. 2D-2F and 7; data not shown). This feature indicates that the commonly observed loss of stem cell functions with cell culture [24] can be accounted for by their asymmetric self-renewal kinetics as proposed previously [15]. After discovery of input factors to simulate published data for mouse embryo fibroblasts, changes in only the input transferred cell number were found to result in PSCK simulations that recapitulate the well-known cell density dependence of CPD curves for mouse embryo fibroblasts [18] (FIGS. 8A and 8B). Previously, there had been no explanation for this effect. This observation indicates that it can be readily explained by the intrinsic asymmetric cell kinetics properties of tissue stem cells.

TABLE 1
Quantitative validations of PSCK-RIFS for tissue stem cell counting and cell kinetics determinations-Comparisons of
PSCK-RIFS tissue stem cell fraction to reported functional determinations^{a}.

| Tissue | | | | | | | |
|---|---|---|---|---|---|---|---|
| Type: | BM-CD34+ | CB-CD34+^{b, c} | CB-CD34−^{b} | Lung | Liver | MSC-Amniotic^{d} | MSC-BM^{c} |
| PSCK-RIFS | 2.6 × 10⁻⁴ ± | 2.4 × 10⁻³ ± | 1.2 × 10⁻⁴ ± 0.0^{‡‡} | 0.04 ± 0.04** | 0.08 ± 0.08** | 3.1 × 10⁻⁴ ± | UD^{f} − 4.6 × 10⁻⁴ ± |
| | 5.5 × 10^{-5‡} | 0.80 − 3^{b§} | | | | 1.4 × 10^{-4‡} | 1.4 × 10^{-4‡} |
| | | 0.08 ± 0.06^{c}* | | | | | |
| Functional | 6.2 × 10⁻⁴ [26] | 1.6 10⁻³ [27], | UD [29], 4 × 10⁻⁵ | 0.13[15] | 0.22 ± 0.13 | N.A.^{h} | N.A. |
| | | 0.025 [28] | −0.001[28] | | [25]^{g} | | |

*p < 0.05;
**p < 0.02;
^{‡}p < 0.01;
^{‡‡}p = 0.001;
^{§}p < 0.0001

TABLE 2
Quantitative validations of PSCK-RIFS for tissue stem cell counting and cell kinetics determinations-Detection of stem
cell-specific cell kinetics effectorsⁱ

| | | | | | |
|---|---|---|---|---|---|
| | | | | BM- | |
| Tissue/Agent: | MSC-BM/Con^{c} | MSC-BM/hPL^{c} | BM-CD34′/Con | CD34′/Xanthosine | BM-CD34′/BCNU |

Parameter
Stem Cells

| | | | | | |
|---|---|---|---|---|---|
| Initial Fraction | 5.6 × 10⁻⁴ (NS)ⱼ | 9.9 × 10⁻⁴ (0.012) | 2.6 × 10⁻⁴ (0.004) | 3.5 × 10⁻³ (0.001) | 1.3 × 10⁻⁴ (0.001) |
| SSR Rate, RS | 0.040 (NS) | 5.2 × 10⁻⁴ (NS) | 1.3 × 10⁻³ (NS) | 3.2 × 10⁻³ (0.037) | 0.0 (NS) |
| Sym CC Time | 9.4 h (0.0033) | 11 h (NS) | 7.8 h (<0.0001) | 9.4 h (NS) | 8.2 h (NS) |
| Asym CC Time | 16 h (NS) | 12 h (NS) | 7.0 h (0.0002) | 6.6 h (NS) | 7.6 (NS) |
| Committed Progenitor Cells | 12 h (0.0034) | 9.8 h (NS) | 6.8 h (<0.0001) | 8.2 h (NS) | 6.4 h (NS) |
| CC Time | | | | | |

a, values are the mean±standard deviation for n=5 determinations; b, data from ref. 30; c, data from ref. 31; d, data from ref. 32; e, data from ref. 24; f, UD, undetectable; g, data not shown; h, N.A., not available; i, values are the mean (p for different than control [Con] value) for n=5 determinations; j, p for Con value different than 0.0; NS, p>0.05.

Four different human tissue cell preparations, for which functional estimates of stem cell fraction were available, were independently evaluated. Functional estimates use measures of tissue cell repopulation or asymmetric self-renewal as indicators of tissue stem cells (See Table 1). In all four cases—bone marrow-derived CD34+-selected cells, umbilical cord-derived CD34+-selected cells, pre-senescent lung cell strains, and expanded liver stem cell strains—the PSCK-RIFS determinations of stem cell fraction were similar to available values reported based on functional estimates.

The most important validations came from comparisons of PSCK-RIFS determinations of human HSC fractions to published HSC fraction data from limiting-dilution SCID mouse repopulating cell (LDSRC) assays. The LDSRC assay, though limited to estimation of only HSC fraction, gives the previously best available estimate of the HSC-specific fraction. The reported LDSRC assay value for human HSCs in CD34+-immunoselected bone marrow cell preparations is 6.2 per 10,000 total cells (Table 1) [26]. Although the PSCK-RIFS determination of 2.6 is unequal to this value with statistical confidence (p<0.0001; Table 1), it is still quite similar numerically. The variance for the reported LDSRC assay determination was not available, making the statistical confidence determination for the difference potentially misleading. Two reports were identified of LDSRC assay determinations of the HSC fraction in CD34+ fractions of human umbilical cord blood (Table 1) [27, 28]. The mean fraction of the reported values was 133±165 per 10,000 total cells (n=2), which was not statistically different than the mean of the two PSCK-RIFS analyses available using reported CPD data [30, 31] (Table 1; 412±549; p=0.56).

Human umbilical cord blood analyses also provided an independent validation of the specificity of PSCK-RIFS stem cell counts. Using published CPD data [30], the PSCK-RIFS analyses showed that HSCs fractionated, as predicted, preferentially to the CD34+ cell fraction in 20-fold excess over the CD34− cell fraction, in which HSCs were at the limit of detection for the analysis (Table 1).

Additional validation came from evaluation of agents reported to alter tissue stem cell proliferation either positively or negatively. As shown in Table 2 the new counting technology detected increases in the stem cell fractions of cultures supplemented with either xanthosine (Xs) or human platelet lysate (hPL). Xs is known to increase the frequency of symmetric stem cell divisions [17]; and hPL has been reported to increase the retention of mesenchymal stem cell (MSC) functions in culture [24].

Consistent with its previously described effects on tissue stem cells [17], Xs increased RS, the rate of symmetric self-renewal (See EXAMPLE 4), for HSCs. The observed Xs-induced increase in HSC number was associated with an increase in RS from undetectable to 0.32% (p=0.037) of HSC divisions. This small change in HSC symmetric self-renewal kinetics is associated with a 10-fold increase in HSC number after only 4 days of culture. The two independently detected increases (RS and HSC number) are mathematically consistent. If only 3 out of 1000 HSCs (0.30%) switched to symmetric self-renewal with an 8-hour generation time, in 96 hours, 12,288 (i.e., ˜10-fold) additional HSCs would be produced. hPL, which had a much smaller effect on human bone marrow-derived MSCs, did not show a significant effect on RS.

In contrast to the stem cell activation by Xs and hPL, the chemotherapeutic agent carmustine, or bis-chloroethylnitrosourea (BCNU), caused a decrease in the determined HSC number within the first four days of culture. This agent has long been thought to be stem cell-toxic because it causes chronic organ failure, including chronic bone marrow failure [33].

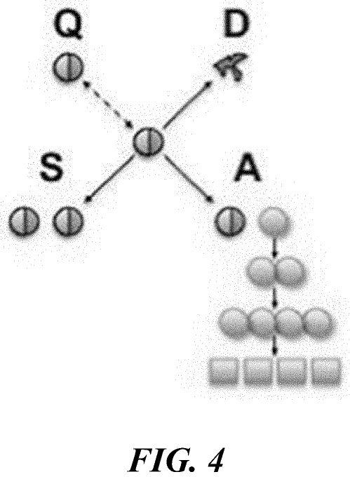

As indicated by the data presented in Tables 1 and 2, the PSCK-RIFS computational simulation technology can determine several important cell kinetics parameters of tissue stem cells, transiently amplifying lineage-committed cells, and terminally-arrested differentiated cells in complex cell preparations (See also FIG. 2C and FIG. 6). Sub-routines of the program allow interrogation of intricate stem cell kinetics properties that have been previously inaccessible. We note the contrast between the cell kinetics of human lung or liver stem cells (FIG. 4D, liver example) and human HSCs or MSCs (FIGS. 4E and 4F, respectively). The exponential inclines observed for human lung (data not shown) and liver stem cells (FIG. 4D, liver) are due to symmetric self-renewal divisions that increase tissue stem cell number. These cell strains also had the highest observed RS values, 0.24±0.16 (p<0.03) and 0.24±0.19 (p<0.05), respectively, compared to CD34+ bone marrow cells, whose RS value was 0.0013±0.0011 and not significantly different than 0.0.

The high RS values signify that approximately 24% of stem cell divisions in these cultures are symmetric self-renewing divisions, which increase stem cell number. The exponential stem cell accumulation inclines (See FIG. 4D) are another manifestation of these self-duplicating stem cell divisions. Within the cell kinetics model postulated, such a high rate of symmetric stem cell division is predicted to delay the rate of stem cell dilution, which would prolong the replicative span of the cultures. Consistent with this prediction, when initiated with the same number of total viable cells (65,000) and passaged on the same schedule (⅓ culture splits every 96 hours; See Materials and Methods), cultures of CD34+-selected bone marrow cells reached division arrest much sooner (12 days) than cultures of lung cell strains or liver stem cell-enriched strains (60-76 days or 60 days, respectively). In contrast, consistent with their smaller RS values, bone marrow-derived human HSCs and MSCs show only rare symmetric self-renewal divisions in culture. This property can explain the characteristic rapid disappearance of bone marrow-derived HSCs and MSCs in serial cell culture.

Discovery of Algorithms for Determining the Initial Stem Cell Fraction from Simple Culture Population Doubling Time Data

Using data outputs from PSCK-RIFS analyses, it is possible to discover simple mathematical algorithms for convenient and rapid determination of the stem cell-specific fraction of tissue cell preparations. Because of the fundamental cell kinetics relationships described in FIG. 1 and FIG. 4, the proliferation rate of any normal human tissue culture is related to the number of stem cells it contains, its stem cell fraction (SCF; FR_NS in FIG. 1, See EXAMPLE 4 and Table 5). A universal measure of cell culture proliferation rate is the population doubling time (PDT). PDT is the time required for a cell culture to double in total cell number. Based on these ideas, it is possible to deduce the number of tissue stem cells in any culture from the culture's PDT, which can be determined by simple counting of the total cells in the culture before and after a short period of culture.

A unique output of PSCK-RIFS analyses, the SCF over time (e.g., See FIG. 3A), makes it possible to define mathematical algorithms that yield the SCF from a culture's simple PDT. This discovery and its development are illustrated in FIG. 3A-3F. The PSCK-RIFS software gives the two required data outputs. The first is the unique SCF throughout a foundational PSCK-RIFS serial culture analysis (FIG. 3A; See also FIG. 6, blue trace). FIG. 3B shows an example of the second output, the corresponding data for the total culture cell number versus days of serial culture. The perpendicular declines in the data are simulations of culture dilutions to start next cultures in the series. The curvilinear inclines from dilution low points reflect the simulated proliferation of the diluted cells. The PDT between any two time points of culture can be calculated with Equation 2:

PDT=[(t2−t1)/ln(N2/N1)] ln 2 (Equation 2)

In Equation 2, t1=the starting time; t2=final time; N1=the initial cell number; and N2=the final cell number.

Computation of the PDT determined from time=0 of serial culture to the first maximum and for each incline in cell number from each successive dilution low point to the next maximum in FIG. 3B can be performed and related to the corresponding SCF values at time=0 and at each culture dilution low point. These data are shown in FIG. 3C and FIG. 3E, respectively, for two different types of tissue stem cells. Both data plots show the predicted characteristic increasing culture PDT as the SCF decreases. The detection of this relationship is an orthogonal validation of the key principle upon with the PSCK-RIFS method is based, namely that tissue stem cells are rate-determining for culture proliferative rate. As shown in FIGS. 3D and 3F, based on these analyses, mathematical conversions that linearize the PDT vs. SCF data into statistically significant descriptions of the data can be found. Similar analyses, based on PDT values calculated with the experimental cell count data for the simulations, yield similar results (data not shown). These transforming PDT:SCF tissue stem cell counting algorithms make it possible to calculate confident estimates of the SCF of the same types of stem cells, when grown under the same culture conditions, from the PDT determined after 72 hours, and potentially less, of cell culture.

### Discussion

The ability to determine tissue stem cell specific-dose using the PSCK-RIFS technology and PDT:SCF algorithms will be of significant value for stem cell transplantation medicine. Past success in stem cell medicine has been primarily in the area of HSC transplantation medicine. Yet, even with HSC transplantation, there is still opportunity for significant improvements in efficacy that would occur with a means to determine HSC-specific dose. A prime example is umbilical cord blood HSC transplantation therapies, for which units fail at a high rate (18-24%), because of insufficient stem cells [34]. Without being able to count HSCs, it has been impossible to know which cord blood units would have insufficient potency. This lack of information is a source of significant morbidity and deaths that could be avoided, if there were a method to routinely identify units with an adequate HSC dose. Transplants with adult bone marrow and mobilized peripheral blood are more reliable, with failure rates of a few percent [35]. However, this lower rate of failure may indicate that often patients receive excess HSCs. Being able to count HSCs would allow identification of scarce donor units that could be used to treat additional patients.

Since the earliest days of tissue stem cell research, mathematical and computer modeling have been employed as tools to investigate the biological properties of tissue stem cells with respect to their earliest progeny, lineage-specific committed progenitor cells [36, 37]. In particular, these approaches were applied to several challenging problems in stem cell biology that were difficult to interrogate by observational or experimental approaches. These more challenging problems included identifying and quantifying tissue stem cells in vivo, discovering the mathematical form of their competing self-renewal and production of committed progenitor cells, and investigating how tissue stem cells and their subtended cellular systems respond to physiological factors and exogenous agents like environmental toxicants and drugs [38-46]. Beyond relevance to important health-related processes in the body (e.g., normal growth and maturation, diseases like cancer, and aging), knowledge of such tissue stem cell properties is crucial to in vitro processes, too, like tissue engineering, stem cell manufacturing, stem cell transplantation medicine, and drug discovery.

The most actively pursued tissue stem cell modeling has focused on the identity and self-renewal properties of hematopoietic stem cells [36, 38-42] or gastrointestinal tract stem cells, including primarily small intestinal epithelial stem cells [12, 47-49] and, to a lesser extent, colonic epithelial stem cells [43]. Recent modeling reports [40-52] focus on the still unsettled question whether tissue stem cells achieve their balance of self-renewal versus tissue cell renewal by stochastic or deterministic division and differentiation programs [41, 53-55]. The mouse has been the major experimental system for evaluating the validity of previously developed mathematical and computer models [41, 42]. As a result, only a dearth of human tissue stem cell modeling studies is available to date. Moreover, the majority of mouse studies are focused on interrogating in vivo processes that may not translate well to meet in vitro needs for improving progress in stem cell medicine.

The described PSCK-RIFS method is grounded on the foundation of tissue cell kinetics concepts from earlier tissue stem cell modeling [12, 13, 15] and experimental observations of human tissue cell strains maintained in serial cultures [11, 56, 57]. The experimental passaging schedules for the PSCK-RIFS approach differ in a fundamental way from those in foundational studies. The earlier studies were focused on maintaining culture proliferation for as long as possible; so that often cultures were held at confluence for extended periods. In marked contrast, cultures for PSCK-RIFS analyses are maintained on serial passage schedules that are designed for rapid declines in cell number with passaging, with cultures achieving a confluent monolayer state for only limited periods early in the serial culture, if ever at all. This strategy accelerates the decline in tissue stem cell number, which is the crucial effect that enables determination of tissue stem cell fraction and specific cell kinetics.

Unlike previous stem cell modeling approaches, whether mathematical or computational, the PSCK-RIFS method was expressly designed to discover previously inaccessible properties of cultured perinatal and postnatal tissue stem cells that constituted crucial gaps in knowledge that limited progress in stem cell research and stem cell medicine. The most significant among these knowledge gaps was a means to determine the tissue stem cell-specific fraction of complex tissue cell preparations, whether experimental samples, manufactured stem cell production lots, or therapeutic treatments. The PSCK-RIFS method can achieve this determination for any cell preparation that will proliferate in culture. Because the initial stem cell fraction is independent of subsequent culture conditions (and changes in the stem cell fraction during culture can be quantified), differences in culture media do not alter the initial stem cell fraction determination.

In contrast, the invented PDT:SCF stem cell counting algorithms developed with the output data of foundational PSCK-RIFS analyses are predicted to be very dependent on the culture conditions under which they were derived. Foundational PSCK-RIFS serial culture analyses discover tissue stem cell factors in the instant conditions, whatever they may be. However, the subsequently derived PDT:SCF algorithms are likely to often only apply to the same type of stem cell preparations under the same culture conditions as those of the foundational analysis. It is already evident that different stem cell types grown under different culture conditions have different PDT:SCF algorithms (Compare FIG. 3D to FIG. 3F). Even the same types of stem cells are predicted to have different PDT:SCF algorithms, if differences in culture conditions significantly alter either their cell kinetics properties or those of other cell types in the culture.

The advantages of the convenience and speed of PDT:SCF algorithms more than outweigh their requirement of specific culture conditions. For the same types of tissue stem cells under the same culture conditions, PDT:SCF algorithms enable timely stem cell counting on the timescale of days or less. Such a capability makes stem cell counting practical for use for laboratory tissue stem cell research, for optimizing and monitoring human cell biomanufacturing, for drug development assays, and for stem cell treatment dose determination.

A major challenge in the development of the PSCK-RIFS technology has been validating its specificity and accuracy. Although the presented validations are sufficient for this stage of development, continued use of the technology in other contexts will be important for its continued validation and improvement. For validation, five different orthogonal approaches were applied. The analyses utilized cumulative population doubling data from original cell culture analyses (5 analyses) as well as data from published serial culture analyses (8 analyses; [18, 24, 30-32]). The use of published cell count data reduced bias in the validations and demonstrated the versatility and facility of the method. For primary validation, PSCK-RIFS determinations were compared to literature reports of the HSC fraction in CD34+-selected human bone marrow cells and umbilical cord blood cells determined by LDSRC assays. The LDSRC assay is the most specific and best available method for estimating the fraction of any tissue stem cell type.

The other four validation bases included comparison to independent determinations of the asymmetrically cycling cell fraction, which is the primary PSCK-RIFS basis for stem cells; analysis of differences in the PSCK-RIFS determination related to tissue stem cell fractionation (CD34 fractionation); analysis of changes in the PSCK-RIFS determination related to supplementation of agents either known (xanthosine) or suspected (hPL) of increasing tissue stem cell proliferation; and analysis of changes in the PSCK-RIFS determination after treatments with agents suspected of toxicity against tissue stem cells (BCNU).

We emphasize here that the basis for the validation of the PSCK-RIFS method and the derived PDT: SCF algorithms is not the use of any pre-existing molecular biomarkers, which are not specific for tissue stem cells. Our independent experimental quantification of HSCs in bone marrow-derived CD34+-selected populations (Table 1) is compared to LDSRC assays in the published literature. Similar validation comparisons, based on PSCK-RIFS determinations using published serial culture data, evaluated changes in HSC number associated with CD34+ fractionation of umbilical cord blood cells (Table 1). None of these validations were based on CD34+ cell counts per se. All other validations used PSCK-RIFS determinations based on serial culture experiments performed in the instant analysis. These included comparisons to independent determinations of asymmetrically self-renewing cells, which is the tissue stem cell-specific cell kinetics factor that is the basis for the PSCK model (Table 1; lung stem cells and liver stem cells). Other validations based on original serial culture data evaluated predicted changes in the determined values of HSCs or MSCs in response to supplementation of cultures with either a positive effector of tissue stem cell self-renewal (Table 2; xanthosine for HSCs) or a tissue stem cell-toxic agent (Table 2; BCNU for HSCs).

These many different independent validations provide a high degree of confidence that the PSCK-RIFS method can provide a faithful estimation of the tissue stem cell-specific fraction of human cell preparations from a variety of source tissues. However, like any biological model-based computational method, the accuracy and precision of determinations depend on the how well the underlying model defines the essential features of the evaluated biological system and the quality of the input data for computation. The PSCK model (FIG. 1) has other input factors, in addition to the main ones that are measured experimentally or discovered by the RIFS software, that are used for the PSCK simulations and the RIFS software search (See EXAMPLE 4). The additional factors, as noted in the main text, are unknown variances that are currently prescribed to fall within a limited “biological range.” Sensitivity analyses were conducted to identify the factors that are the more significant determinants in PSCK simulations. However, presently the possibility cannot be excluded that the input variance assumptions may sometimes be incorrect and impact the accuracy of PSCK-RIFS determinations. Of course, to the extent that any significant feature of the model is incorrect, the accuracy and, potentially, the precision of stem cell fraction determinations will be affected. As the first general method for quantifying tissue stem cells specifically, the best future test of the PSCK-RIFS method will be the collective outcomes of its widespread use for investigation of applications in stem cell science and stem cell medicine. For example, the finding that the stem cell dose determinations by the PSCK-RIFS method are predictive of clinical outcomes in stem cell transplantation medicine would be an empirical validation of its value and ability to provide reliable tissue stem cell quantitation.

Beyond quantitative validation that a variety of tissue stem cells can be counted specifically, the statistical confidence of the accuracy of PSCK-RIFS determinations was also evaluated. Indicative of the statistically significant p-values for determinations in Tables 1 and 2, the 95% confidence intervals—provided in Table 3 of PSCK-RIFS stem cell fraction determinations over a diverse range of tissue stem cell types—show a high degree of resolution. A similar degree of statistical confidence is observed for other cell kinetics factors determined by the PSCK-RIFS method. The narrow range of the confidence intervals reveals that the five 1000-cycle RIFS search analyses (See EXAMPLE 4), used to discover the stem cell fraction and other cell kinetics factors, yield a unique and single solution within the experimental error of the analysis. This quantitative characteristic is another indication of the quality of the PSCK-RIFS method.

TABLE 3
Analyses of the confidence intervals of PSCK-RIFS
tissue stem cell fraction determinations¹.

| | Tissue Stem Cell Fraction | |
|---|---|---|
| Tissue Type | (per 10,000 total cells) | 95% Confidence Interval |
| BM-CD34+ | 2.6 | 2.0-3.2 |
| CB-CD34+ | 24 | 18-30 |
| Lung | 420 | 12-720 |
| Liver | 812 | 200-1420 |
| MSC-Amniotic | 3.1 | 1.4-4.8 |
| MSC-BM | 4.6 | 2.9-6.3 |

In summary, the PSCK-RIFS technology and the new invention of PDT: SCF algorithms are a solution to the long-standing challenge of specific and accurate quantification of adult tissue stem cells. The technologies can be applied to tissue stem cells in complex cell preparations, including stem cell experiments, stem cell production cultures, and stem cell treatments. Adoption of the new method will accelerate progress in stem cell science and stem cell medicine by addressing longstanding unmet needs like determining the dose and quality of therapeutic stem cells. The technology can also be applied for evaluation of the effects of varied compounds on tissue stem cells and for optimizing the production of stem cells for cell therapy. Finally, the PSCK-RIFS technology is a new tool for evaluation of new classes of molecular biomarkers with potential to specifically identify and quantify adult tissue stem cells directly [3].

### CONCLUSIONS

A first method for specific and accurate counting of perinatal and postnatal tissue stem cells is described. The method uses computer simulation of a probabilistic stem cell kinetics model—of how total cells are produced from tissue stem cells in culture—to discover previously inaccessible tissue stem cell kinetics factors, including among them the stem cell-specific fraction. The ability of the described Probabilistic Stem Cell Kinetics-Random Input Factor Searching (PSCK-RIFS) software to determine the stem cell-specific fraction of a variety of different human tissue cell preparations was validated by several different comparisons to the best available estimates of stem cell-specific fraction. These comparisons included estimates of human hematopoietic stem cell fraction by limiting-dilution SCID mouse repopulating cell assays and effects of tissue stem cell-active agents. A major advance enabled by PSCK-RIFS analyses was the invention of Population Doubling Time: Stem Cell Fraction (PDT:SCF) algorithms that allow convenient and rapid determination of tissue stem cell fraction from simple culture population doubling time data. The new tissue stem cell counting technologies will enable stem cell-specific dose determination for stem cell transplantation medicine for the first time. Such capability is predicted to yield many advances in stem cell transplantation medicine, as well as tissue stem cell research, cell biomanufacturing, drug development, and environmental health science.

### REFERENCES

1. Ivanovic Z. Hematopoietic stem cells in research and clinical applications: The “CD34 issue.” World J Stem Cells. 2010; 2: 18-23.
2. Sherley J L. Principles for targeting adult tissue stem cells to achieve durable gene and gene-editing therapeutics. Cambridge: Cambridge University Press; 2018. p. 285-300.
3. Sherley J L. Dose determination for stem cell medicine: A need whose time has come. Amsterdam: Elsevier; 2018. p. 383-397.
4. Caplan A. Mesenchymal stem cells: Time to change the name! Stem Cells Transl Med. 2017; 6: 1441-1445.
5. Sherley J L. Accelerating progress in regenerative medicine by advancing distributed stem cell-based normal human cell biomanufacturing. Pharm Anal Acta. 2014; 5: 286.
6. Rich I N. Improving quality and potency testing for umbilical cord blood: A new perspective. Stem Cells Transl Med. 2015; 4: 967-973.
7. Purton L E, Scadden D T. Limiting factors in murine hematopoietic stem cell assays. Cell Stem Cell. 2007; 1: 263-270.
8. Patterson J, Moore C H, Palser E, Hearn J C, Dumitru D, Harper H A, et al. Detecting primitive hematopoietic stem cells in total nucleated and mononuclear cell fractions from umbilical cord blood segments and units. J Transl Med. 2015; 13: 94.
9. Sherley J L. Asymmetric self-renewal: The mark of the adult stem cell. London: Imperial College Press; 2005. p. 21-28.
10. Sherley J L. New cancer diagnostics and therapeutics from a 9th “hallmark of cancer”: Symmetric self-renewal by mutated distributed stem cells. Expert Rev Mol Diagn. 2013; 13: 797-810.
11. Hayflick L. The limited in vitro lifetime of human diploid cell stains. Exp Cell Res. 1965; 37: 614-636.
12. Potten C S, Morris R J. Epithelial stem cells in vivo. J Cell Sci Suppl. 1988; 10: 45-62.
13. Loeffler M, Potten C S. Stem cells and cellular pedigrees-A conceptual introduction. San Diego: Harcourt Brace & Co.; 1997. p. 1-28.
14. Pare J F, Sherley J L. Biological principles for ex vivo adult stem cell expansion. San Diego: Elsevier, Inc.; 2006. p. 141-171.
15. Rambhatla L, Bohn S A, Stadler P B, Boyd J T, Coss R A, Sherley J L. Cellular senescence: Ex vivo p53-dependent asymmetric cell kinetics. J Biomed Biotech. 2001; 1: 27-36.
16. Merok J R, Sherley J L. Breaching the kinetic barrier to in vitro somatic stem cell propagation. J Biomed Biotech. 2001; 1: 24-26.
17. Lee H S, Crane G G, Merok J R, Tunstead J R, Hatch N L, Panchalingam K, et al. Clonal expansion of adult rat hepatic stem cell lines by suppression of asymmetric cell kinetics (SACK). Biotechnol Bioeng. 2003; 83: 760-771.
18. Todaro G J, Green H. Quantitative studies of the growth of mouse embryo cells in culture and their development into established lines. J Cell Biol. 1963; 17: 299-313.
19. Majore I, Moretti P, Stahl F, Hass R, Kasper C. Growth and differentiation properties of mesenchymal stromal cell populations derived from whole human umbilical cord. Stem Cell Rev. 2011; 7: 17-31.
20. Duncan E L, Reddel R R. Genetic changes associated with immortalization. A review. Biochemistry (Mosc). 1997; 62: 1263-1274.
21. Rogan E M, Bryan T M, Hukku B, Maclean K, Chang A C, Moy E L, et al. Alterations in p53 and p16INK4 expression and telomere length during spontaneous immortalization of Li-Fraumeni syndrome fibroblasts. Mol Cell Biol. 1995; 15: 4745-4753.
22. Harvey D J, Levine A J. P53 alteration is a common event in the spontaneous immortalization of primary BALB/c murine embryo fibroblasts. Genes Dev. 1991; 5: 2375-2385.
23. Sherley J L, Stadler P B, Johnson D R. Expression of the wild-type p53 antioncogene induces guanine nucleotide-dependent stem cell division kinetics. Proc Natl Acad Sci USA. 1995; 92: 136-140.
24. Heathman T R, Stolzing A, Fabian C, Rafiq Q A, Coopman K, Nienow A W, et al. Scalability and process transfer of mesenchymal stromal cell production from monolayer to microcarrier culture using human platelet lysate. Cytotherapy. 2016; 18: 523-535.
25. Panchalingam K, Noh M, Huh Y H, Sherley J L. Distributed stem cell kinetotoxicity: A new concept to account for the human carcinogenicity of non-genotoxic toxicants. London: Royal Society of Chemistry; 2016. p. 250-279.
26. Ziegler B L, Valtieri M, Porada G A, De Maria R, Muller R, Masella B, et al. KDR receptor: A key marker defining hematopoietic stem cells. Science. 1999; 285: 1553-1558.
27. De Wynter E A, Emmerson A J B, Testa N G. Properties of peripheral blood and cord blood stem cells. Baillieres Best Pract Res Clin Haematol. 1999; 12: 1-17.
28. Takahashi M, Matsuoka Y, Sumide K, Nakatsuka R, Fujioka T, Kohno H, et al. CD133 is a positive marker for a distinct class of primitive human cord blood-derived CD34-negative hematopoietic stem cells. Leukemia. 2014; 28: 1308-1315.
29. Kim D K, Fujiki Y, Fukushima T, Ema H, Shibuya A, Nakauchi H. Comparison of hematopoietic activities of human bone marrow and umbilical cord blood CD34 positive and negative cells. Stem Cells. 1999; 17; 286-294.
30. Engelhardt M. Telomerase regulation, cell cycle, and telomerase stability in primitive hematopoietic cells. Blood. 1997; 90: 182-193.
31. Durand B, Migliaccio G, Yee N S, Eddleman K, Huima-Byron T, Migliaccio A R, et al. Long-term generation of human mast cells in serum-free cultures of CD34+ cord blood cells stimulated with stem cell factor and interleukin-3. Blood. 1994; 84: 3667-3674.
32. Pratama G, Vaghjiani V, Tee J Y, Liu Y H, Chan J, Tan C, et al. Changes in culture expanded human amniotic epithelial cells: Implications for potential therapeutic applications. PLoS ONE. 2011; 6: e26136.
33. Marsh J C. The effects of cancer chemotherapeutic agents on normal hematopoietic precursor cells: A review. Cancer Res. 1976; 36: 1853-1882.
34. Olsson R, Remberger M, Schaffer M, Berggren D M, Svahn B M, Mattsson J, et al. Graft failure in the modern era of allogeneic hematopoietic SCT. Bone Marrow Transp. 2013; 48: 537-543.
35. Wolff S N. Second hematopoietic stem cell transplantation for the treatment of graft failure, graft rejection, or relapse after allogeneic transplantation. Bone Marrow Transpl. 2002; 29: 545-552.
36. Till J E, McCulloch E A, Siminovitch L. A stochastic model of stem cell proliferation, based on the growth of spleen colony-forming cells. Proc Natl Acad Sci USA. 1964; 5: 29-36.
37. Newton C M. Computer simulation of stem-cell kinetics. Bull Math Biophys. 1965; 27: 275-290.
38. Niewisch H, Vogel H, Matioli G. Concentration, quantitation, and identification of hemopoietic stem cells. Proc Natl Acad Sci USA. 1967; 58: 2261-2267.
39. Nakahata T, Gross A J, Ogawa M. A stochastic model of self-renewal and commitment to differentiation of the primitive hemopoietic stem cells in culture. J Cell Physiol. 1982; 113: 455-458.
40. Ema H, Takano H, Sudo K, Nakauchi H. In vitro self-renewal division of hematopoietic stem cells. J Exp Med. 2000; 192: 1281-1288.
41. Roeder I, Kamminga L M, Braesel K, Dontje B, de Haan G, Loeffler M. Competitive clonal hematopoiesis in mouse chimeras explained by a stochastic model of stem cell organization. Blood. 2005; 105: 609-616.
42. Glauche I, Cross M, Loeffler M, Roeder I. Lineage specification of hematopoietic stem cells: Mathematical modeling and biological implications. Stem Cells. 2007; 25: 1791-1799.
43. Boman B M, Fields J Z, Bonham-Carter O, Runquist O A. Computer modeling implicates stem cell overproduction in colon cancer initiation. Cancer Res. 2001; 61: 8408-8411.
44. Potten C S, Booth C, Hargreaves D. The small intestine as a model for evaluating adult tissue stem cell drug targets. Cell Prolif. 2003; 36: 115-129.
45. Walters K. Colonic stem cell data are consistent with the immortal model of stem cell division under non-random strand segregation. Cell Prolif. 2009; 42: 339-347.
46. Barker N, van Oudenaarden A, Clevers H. Identifying the stem cell of the intestinal crypt: Strategies and pitfalls. Cell Stem Cell. 2012; 11: 452-460.
47. Loeffler M, Birke A, Winton D, Potten C. Somatic mutation, monoclonality and stochastic models of stem cell organization in the intestinal crypt. J Theor Biol. 1993; 160: 471-491.
48. Podolsky D K. Regulation of intestinal epithelial proliferation: A few answers, many questions. Am J Physiol. 1993; 264: G179-G186.
49. Marshman E, Booth C, Potten C S. The intestinal epithelial stem cell. BioEssays. 2002; 24: 9198.
50. Snippert H J, Van Der Flier L G, Sato T, Van Es J H, Van Den Born M, Kroon-Veenboer C, et al. Intestinal crypt homeostasis results from neutral competition between symmetrically dividing Lgr5 stem cells. Cell. 2010; 143: 134-144.
51. Simons B D, Clevers H. Strategies for homeostatic stem cell self-renewal in adult tissues. Cell. 2011; 145: 851-862.
52. Klein A M, Simons B D. Universal patterns of stem cell fate in cycling adult tissues. Development. 2011; 138: 3103-3111.
53. Vogel H, Niewisch H, Matioli G. The self-renewal probability of hemopoietic stem cells. J Cell Physiol. 1968; 72: 221-228.
54. Müller-Seiberg C E, Cho R H, Thoman M, Adkins B, Sieburg H B. Deterministic regulation of hematopoietic stem cell self-renewal and differentiation. Blood. 2002; 100: 1302-1309.
55. Vogel H, Niewisch H, Matioli G. Stochastic development of stem cells. J Theoret Biol. 1969; 22: 249-270.
56. Smith J R and Hayflick L. Variation in the life-span of clones derived from human diploid cell strains. J Cell Biology. 1974; 62: 48-53.
57. Macieira-Coelho A, Taboury F. A re-evaluation of the changes in proliferation in human fibroblasts during ageing in vitro. Cell Tissue Kinet. 1982; 15: 213-224.

### Example 3

### Development of Simple Algorithms for Rapid Determination of the Hematopoietic Stem Cell-Specific Fraction of Human Umbilical Cord Blood Units

The human umbilical cord blood (UCB) banking industry has needed a means for determining the hematopoietic stem cell (HSC)-specific fraction of cord blood units since its beginning. Because of their small blood volumes, generally cord blood units have insufficient HSCs to achieve effective bone marrow rescue in adults. The same limitation causes a high rate of CB HSC transplant failures in children. For many years, the lack of an effective method for quantifying HSCs in CB units has made CB HSC transplantation medicine a morbid waiting game. Children and their families wait for months in anguish not knowing if their transplant had sufficient stem cells. Neither of the two most widely used assays for estimating CB HSC fraction is effective. The colony forming unit (CFU) assay, which is widely used as a test of the progenitor cell activity of CB units, does not give a measure of HSC-specific dose, and it does not predict the potency of HSC units. The limiting-dilution SCID mouse repopulating cell (LDSRC) assay does provide a specific estimate of HSCs. However, it takes too long, is too impractical, and is too expensive for use as a routine method for determining the HSC-specific dose of CB units. Other methods like flow cytometry and metabolic activity assays are more time-efficient, have higher precision, and are more cost-effective. However, like CFU assays, they lack the essential specificity needed to distinguish HSCs from short-lived committed progenitor cells. Recently, Asymmetrex developed a computational simulation method, the TORTOISE Test™, that achieves specific and accurate quantification of postnatal and perinatal tissue stem cells. Now well validated, the TORTOISE Test™ technology provides reliable, specific, and accurate counting of CB HSCs for the first time. Though effective, the TORTOISE Test™ counting technology is not ideally convenient for routine use. It requires production of data from long periods (weeks) of serial cell culture and cell counting followed by computational software analysis.

Herein is the description of an invention that provides a solution to the earlier challenges experienced with use of the TORTOISE Test™ technology. Using the foundational computational output of the TORTOISE Test™ software, simple tissue stem cell counting algorithms were defined. The new algorithms can be applied to simple population doubling time data to determine the HSC-specific fraction of a sample of cord blood cultured for only a few days. The new “population doubling time:stem cell fraction (PDT:SCF) algorithms” provide the long-needed rapid and convenient method for quantifying the dose of HSCs in therapeutic cord blood units.

### Introduction

Asymmetrex recently developed the first ever technology for specific and accurate counting human perinatal and postnatal tissue stem cells (1-3). Quantification of the tissue stem cell-specific fraction has essential applications in several important biomedical disciplines and industries. Throughout tissue cell research, and tissue stem cell research in particular, quantification of tissue stem cell number will improve the design and interpretation of experimental investigations. In cell biomanufacturing, quantification of tissue stem cells is needed to guide the process engineering of tissue cell production processes. In pharmaceutical and biopharmaceutical development, the ability to detect specific drug candidate effects on tissue stem cells offers many new advantages for accelerating drug development and reducing drug development costs. There are even applications in environmental health science towards developing animal-free tests for stem cell-toxic environmental toxicants. As a form of medicine, both the development and practice of stem cell transplantation medicine will be greatly improved and accelerated by the implementation of stem cell-specific dosage.

Improvements in stem cell medicine are particularly relevant to hematopoietic stem cell transplantation (HSCT) with umbilical cord blood (CB). CB HSCTs fail at a high rate (18-24%), because of insufficient stem cells (4). Without being able to count HSCs specifically, previously, it was impossible to know which cord blood units had insufficient potency. This lack of information is the cause of significant morbidity and deaths that could be avoided, if there were a method to routinely identify units with an adequate HSC dose. Transplants with adult bone marrow and mobilized peripheral blood are more reliable, with failure rates of a few percent (5). There is a distinct possibility that this lower rate of failure indicates that often patients obtaining bone marrow HSCT receive excess HSCs. In the future, quantifying the dose of HSCs could allow identification of scarce donor units that could be used to treat additional patients.

Umbilical CB HSCs have been a major focus for the validation Asymmetrex's TORTOISE Test™ tissue stem cell counting technology (3). The technology yields HSC fraction determinations for CD34⁺-selected CB cells that are statistically equivalent to the HSC-specific fraction determined from limiting-dilution SCID mouse repopulating cell (LDSRC) assays. Moreover, TORTOISE Test™ quantification of samples from CD34⁺ fractionation of CB confirms the stem cell specificity of the method. The TORTOISE Test™ quantifies 20-fold more HSCs in the CD34⁺ cell fraction; and HSCs quantified in the CD34⁻ cell fraction approach the limit of detection (3).

Despite the clear value and advantages of determining the HSC-specific fraction of CB, adoption of the TORTOISE Test™ since its introduction has been slow. This response may reflect in part the technical requirements for performing the test. The TORTOISE Test™ quantification basis is the highly unique asymmetric self-renewal of tissue stem cells like CB HSCs. The method uses a cell kinetics modeling approach to quantify both asymmetrically self-renewing tissue stem cells and tissue stem cells switching to symmetric self-renewal. The modeling requires total cell count data from long-term serial culture of the evaluated tissue cell sample. Once these data are in hand, the computational TORTOISE Test™ software can be applied to determine the specific stem cell fraction (SCF) of the starting tissue cell sample. The TORTOISE Test™ analysis is quite powerful in giving access to both stem cell and non-stem cell kinetics factors that were previously inaccessible (e.g., stem cell cycle time, stem cell symmetric self-renewal rate). However, achieving these advantages requires several weeks of intensive cell culture and cell counting, constituting a protracted and expensive assay.

In the present example details how noted impediments to the use of the TORTOISE Test™ stem cell quantification technology have been alleviated. The computational output of the foundational TORTOISE Test™ analyses provides the ability to relate the SCF, throughout the period of serial culture, to the culture population doubling time (PDT) at any time during the serial culture as well. Mathematical interrogation of relationships between these two sets of unique cell kinetics data revealed the existence of simple mathematical algorithms that can be used to determine the SCF of a sample after determination of the culture PDT from a short growth period. The development of such “PDT:SCF algorithms” for HSCs in whole unfractionated human umbilical CB is described herein. The new algorithms promise to be an important new facile research and medical tool for improving CB HSCT medicine.

### Methods

Two duplicate aliquots of whole unfractionated human umbilical CB were cultured as triplicate suspension cultures with conventional commercial hematopoietic cell culture medium. Every 72 hours, one third of the cultures was passaged to start a new respective culture with the same total medium volume. At each passage, the total number of live cells and dead cells in cultures (based on trypan blue dye exclusion) were determined and recorded. When cultures showed no increase in total cell number after two consecutive passages, the analysis was ended. Cell count data were analyzed with the TORTOISE Test™ software to determine the initial SCF for each of the two duplicate CB cell aliquots. Computational SCF and PDT data were used to define PDT:SCF algorithms.

95% confidence intervals were based on Student's 2-tailed t-test for a single value of 0.0.

### Results

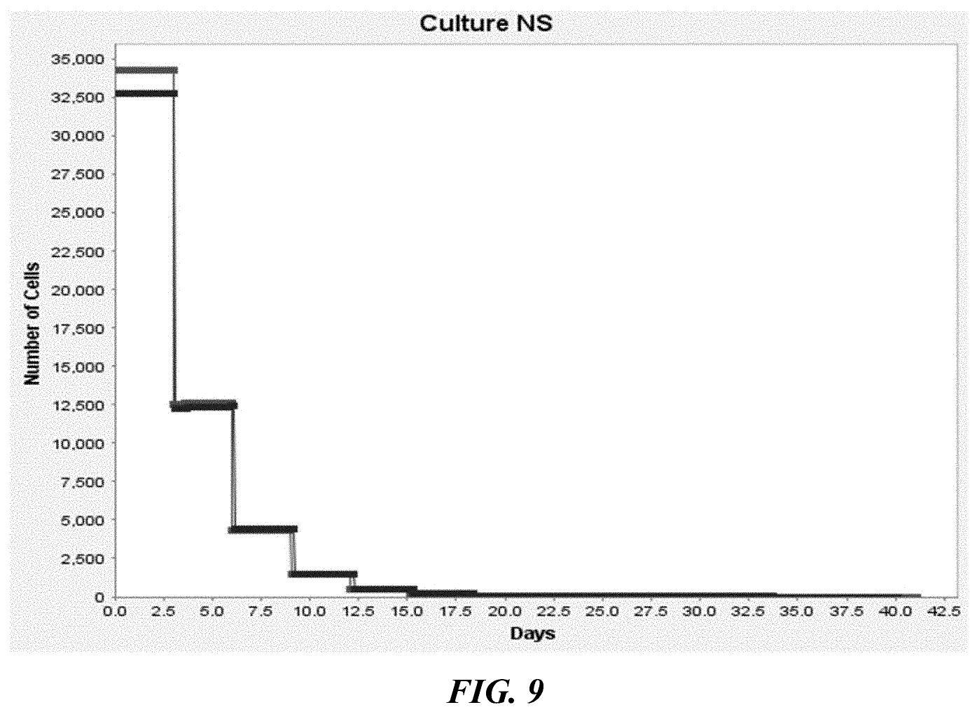

FIG. 9 provides an example of the quantification of specific HSC numbers computed by the TORTOISE Test™ software using simple cell count data from the serial culture of whole unfractionated umbilical CB. The data show the characteristic decline in stem cell number with serial culture (3,6,7). This feature of tissue stem cell kinetics is due to the continuation of their unique asymmetric self-renewal division during cell culture.

The number of total cells is known during the same period of serial culture both empirically from experimental cell count data and computationally from the TORTOISE Test™ software. These two different sources of data show excellent agreement (data not shown). In addition, the software provides projections of the total cell count data continuously between the points of the experimental data. These computations make it possible to compute the HSC-specific fraction (i.e., SCF) at all times during the serial culture.

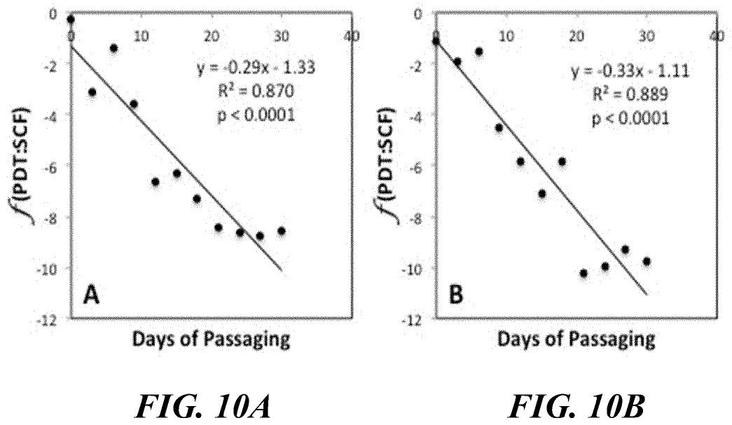

The software can also compute the instantaneous PDTs of the cell cultures throughout the serial culture period. We interrogated mathematical relationships between the PDT data and the SCF data. These analyses yielded simple algorithms that require only simple, short-term, PDT data from cultures to compute their initial HSC-specific fraction (fin FIG. 10A-10B).

Table 4 compares the HSC fractions determined with the new PDT algorithms (See FIG. 10) to the original HSC fractions (per 10,000 mononuclear cells, MNCs) determined with the foundational TORTOISE Test™ method. As can be seen, within the statistical confidence of the methods, the determinations are equivalent.

TABLE 4
Comparison of Umbilical Cord Blood HSC Fraction Determined
by Foundational TORTOISE Test ™ Counting
and PDT:SCF Algorithm Counting

| | Foundation Count | |
|---|---|---|
| | [mean ± SD per | |
| | 10,000 MNCs (95% CI)] | PDT:SCF Algorithm Count |
| Trial A | 35 ± 15 (16-54) | 44 ± 3 (37-51) |
| Trial B | 30 ± 17 (9-51) | 72 ± 13 (40-104) |

PDT:SCF algorithms, like the ones described here for quantifying the specific HSC fraction for whole CB, were also recently described for CD34⁺-selected CB cells (3). Similar algorithms have also be reported for lung stem cells (3) and developed for mesenchymal stem cells (data not shown). The new algorithms remove the practical challenges experienced with the foundational TORTOISE Test™ technology. Now, once a foundational analysis is completed, a PDT:SCF algorithm can be developed that, thereafter, requires only PDT data from a brief period of cell culture to determine the SCF of a tissue cell sample. For the same source of tissue cultured under the same conditions, a single PDT:SCF algorithm can be used repeatedly for rapid and inexpensive determination of SCF.

An important future assessment of PDT:SCF algorithms and their foundational TORTOISE Test™ method will be evaluation of whether the SCF determinations are predictive of the clinical outcomes of tissue stem cell transplantation therapies. Certainly, they are predictive in mice based on the earlier validations for the TORTOISE Test™. The SCFs for CD34⁺-fractionated CB is highly correlated with HSC engraftment efficiency in SCID mice. CB HSCT therapy is a similarly ideal clinical indication for evaluating this question with human patients. Asymmetrex is now planning a first retrospective analysis to determine how well the HSC fraction of CB determined with PDT:SCF algorithms predicts the clinical outcome of CB HSCT.

### CONCLUSIONS

In the present example, a new invention for rapid, inexpensive, specific, and accurate determination of the number and dose of hematopoietic stem cells in umbilical cord blood is described. The described PDT:SCF (Population Doubling Time: Stem Cell Fraction) algorithms require only simple, short-term cell culture data to quantify the stem cell fraction of diverse human postnatal and perinatal tissues, including the hematopoietic stem cell fraction of umbilical cord blood. PDT:SCF algorithms are derived with Asymmetrex's TORTOISE Test™ software, the only technology available for routine, specific and accurate quantification of tissue stem cells. The new stem cell counting algorithms provide a convenient means to obtain a highly important cellular determinant for many applications in cell science, biomanufacturing, drug development, environmental health science, and medicine.

### REFERENCES

1. Sherley J L. Methods for determining the effects of an agent on tissue stem cells. U.S. Pat. No. 9,733,236. 2017.
2. Sherley J L. Methods for determining the effects of an agent on tissue stem cells. U.K. Patent No. GB2529921. 2019.
3. Dutton R, Abdi F, Minnetyan L and Sherley J L. A computational simulation technology for specific counting of perinatal and postnatal human tissue stem cells for transplantation medicine. OBM Transplantation. 2020; in press.
4. Olsson R et al. Graft failure in the modern era of allogeneic hematopoietic SCT. Bone Marrow Tramp. 2013; 48: 537-543.
5. Wolff S N. Second hematopoietic stem cell transplantation for the treatment of graft failure, graft rejection, or relapse after allogeneic transplantation. Bone Marrow Transpl. 2002; 29: 545-552.
6. Rambhatla L et al. Cellular senescence: ex vivo p53-dependent asymmetric cell kinetics. J. Biomed. Biotech. 2001; 1: 27-36.
7. Sherley J L. Accelerating progress in regenerative medicine by advancing distributed stem cell-based normal human cell biomanufacturing. Pharm. Anal. Acta. 2014; 5: 286. doi: 10.4172/2153-2435.1000286.

### Example 4

### PSCK-RIFS Operations Used to Develop SCF vs Time Data Profiles

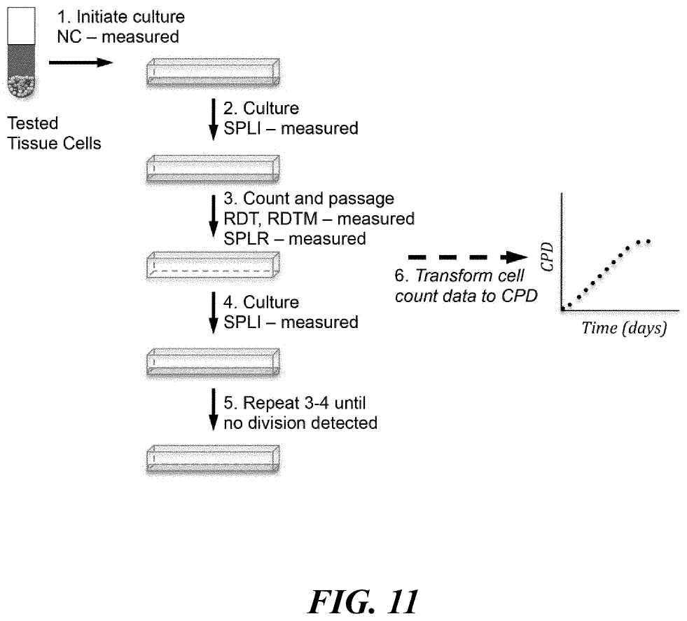

FIGS. 1 and 11 outline the application of the PSCK-RIFS software. A tissue cell preparation of interest is first counted (including viable cell determination; NC in Table 5) and used to initiate serial cultures, typically in triplicate.

At an experimenter defined interval (SPLI in Table 5), cultures are counted—including viable cell count—and a constant fraction of the cells or number of the cells is transferred to a new culture dish of the same type (FIG. 11). The PSCK software is designed to be versatile for accommodating different formats for the culture time period and/or the number of cells transferred. The total cell count data are transformed into plots of cumulative population doublings (CPD); and the viable cell fraction is used to estimate the dead cell fractions for transient cells (RDT in Table 5) and terminal cells (RDTM in Table 5), which are similarly very abundant during most of the culture period.

FIG. 1 depicts the various inputs that are used by the PSCK software to calculate CPD curve simulations for the period defined by the experimental CPD data. Using the cellular model illustrated in FIG. 1, the software inputs measured values (“m” superscripted in FIG. 1) and discovered factors (“d” superscripted in FIG. 1) obtained from the RIFS software. An important aspect of the PSCK-RIFS method is that there is no underlying mathematical algorithm. Instead, simulations are developed purely by a computational counting program. They begin with the number of total tissue cells dictated by the experimental data (i.e., NC). The starting cellular distribution with respect to stem cell fraction, transiently amplifying cell fraction, and terminally differentiated cell fraction can be varied among different idealized patterns representing disrupted tissue structures or cultured primary cell strains (e.g., exponential distribution, uniform distribution). Then the RIFS program feeds known and discovered input factors into PSCK simulations. The PSCK simulation program computes the total cell number produced during each culture interval according to the model and performs randomized splits at the end of each culture interval for the number of passages specified by the investigator. These data are transformed into simulated CPD data plots.

The quality of input factor sets discovered by PSCK-RIFS is assessed quantitatively. Each PSCK-RIFS analysis is performed for 1000 cycles. Each cycle includes RIFS selection of unknown factors, followed by generation of replicate PSCK simulations using the selected unknown factors, followed by comparison of the replicate PSCK CPD simulations to the source experimental replicate CPD data. A quantitative quality score was devised for these comparisons. The quality score is a 2-dimensional root mean square error (RMSE) measure of how close the combined magnitude and variance of the replicate CPD simulations about the experimental mean CPD data approximate the respective features of the experimental replicate CPD data about the experimental mean CPD data. The best quality score is 0.0. Ideally, quality scores≤0.5 are preferred. However, scores≤1.0 are acceptable.

TABLE 5
Probabilistic Stem Cell Kinetics (PSCK) Model Input Factors.
Acronym, input factor

| | |
|---|---|
| NC, number of initiating viable cells - measured | RDTM, terminal cell death fraction - estimated |
| measured COV; normal distribution | from total viability estimated COV; |
| | normal distribution |
| FR_NS, initial stem cell fraction | SWAGE, fractional cell age for RA to RS |
| estimated COV; normal distribution | switching = 0.67 |
| | (RA, asymmetric stem cell fraction = 1--RS---RQS--RDS) |
| NCMAX, maximum cell number capacity - | GTA, asymmetric stem cell generation time |
| measured estimated COV; normal | estimated COV; log normal distribution |
| distribution | |
| RS, symmetric stem cell fraction | GTS, symmetric stem cell generation time |
| estimated COV; normal distribution | estimated COV; log normal distribution |
| RQS, quiescent stem cell fraction | GTT, transient cell generation time |
| estimated COV; normal distribution | measured COV; log normal distribution |
| RDS, stem cell death fraction | TDN, transient cell turnover division number = |
| estimated COV; normal distribution | the number of divisions in an asymmetrically |
| estimated COV; normal distribution | produced committed cell lineage before terminal |
| | cell cycle arrest estimated COV; normal distribution |
| RDT, transient cell death fraction - estimated | ITDN, initiating TDN = average number of |
| from total viability estimated COV; normal | division levels populated in the initial cell |
| distribution | population estimated COV; normal distribution |
| SPLR, split ratio - measured | SPLI, split interval - measured |
| estimated COV; normal distribution | measured COV; normal distribution |
| SPLTMN, minimum number of cell required for | RS Cell Density (RSCD) Factor: |
| a split - prescribed; usually 0.0 | RS_{CD} = [(CDK)(CD) + RSₒ] ≤ RSMAX; CD |
| | input as fraction of NCMAX |
| RSMAX, maximum RS due to cell density | PK, purine equilibrium constant |
| estimated COV; normal distribution | estimated COV; normal distribution |
| RS Purine (RS_{P}) Factor: | PURINE, purine concentration (mM) - |
| RS_{P} = (PRSMAX)(PURINE)/(PK + PURINE) | measured estimated COV; normal |
| | distribution |
| CDK, cell density rate constant | PRSMAX, maximum RS due to purine |
| estimated COV; normal distribution | concentration |

For all studies presented, “n=5”, refers to 5 independent, 1000-cycle PSCK-RIFS analyses to discover the sets of unknown input factors. For each discovered input factor, values from the 5 independent PSCK-RIFS analyses were averaged to estimate statistical significance and to make comparisons among different culture conditions. The replicate analyses reported have PSCK-RIFS quality scores<0.5; and all presented analyses have quality scores<1.0.

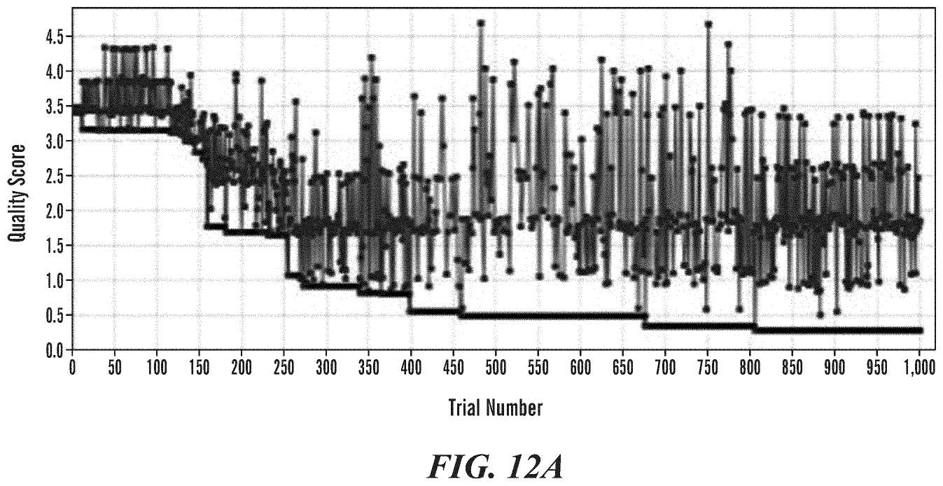

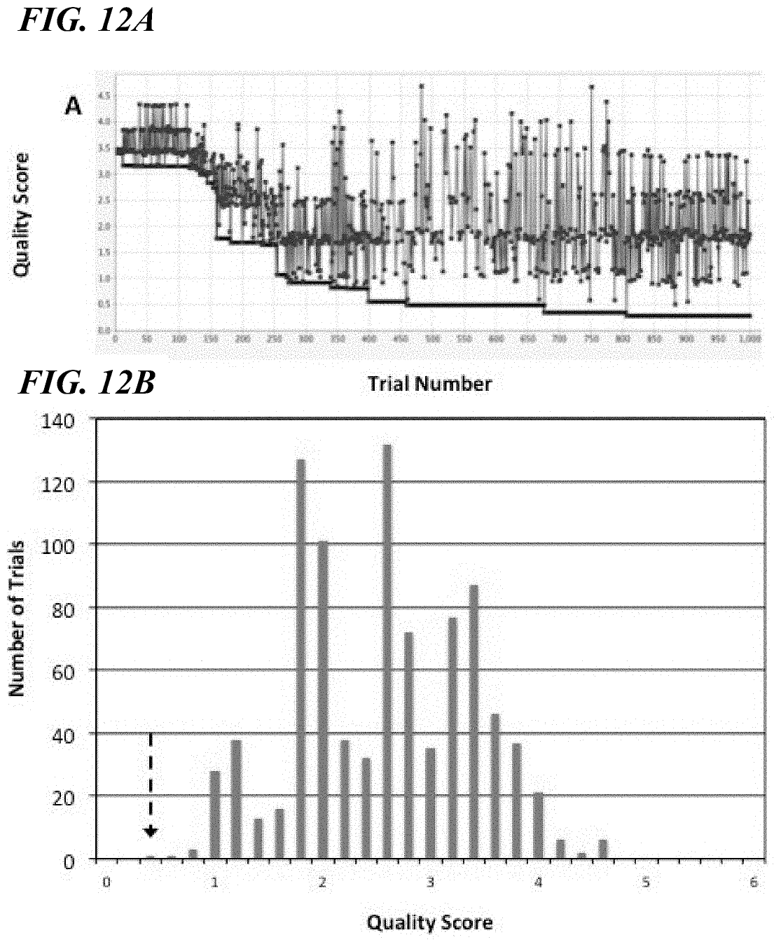

Two analyses performed routinely illustrate that the discovered cell kinetics factor sets are discrete. FIGS. 12A and 12B provide an example of a typical history for a 1000-cycle RIFS search for an optimal PSCK factor set. The y-axis gives the quality score as defined above for the ability of factor sets at the end of each search cycle to simulate a triplicate experimental CPD dataset using the PSCK stem cell-based growth simulation program. The x-axis indicates the progression of search iterations from 1 to 1000. The RIFS program keeps track of factor sets that give the best (lowest 2-dimensional RIVISE score described above) quality scores and uses this information for subsequent searches (i.e., a machine learning basis). The bold black line indicates this retained and compared minimal quality score factor set. FIG. 12B shows the frequency distribution of quality scores (y-axis, number of searches; x-axis quality score) from the search history in FIG. 12A. The normal character of this distribution indicates a robust model structure; and it shows that the minimal quality score factor set discovered is a significantly discrete set of factors (arrow).

### Example 5

### Tissue Stem Cell Fraction Half Life

The methods provided herein enable the calculation of a tissue cell preparation's SCF (and equivalent stem cell-specific dosage) by inputting its PDT into a defined kinetic stem cell (KSC) counting algorithm. Interestingly, the method provided herein can be used to determine the exponential decay half-life of the SCF for a cultured tissue cell preparation containing tissue stem cells.

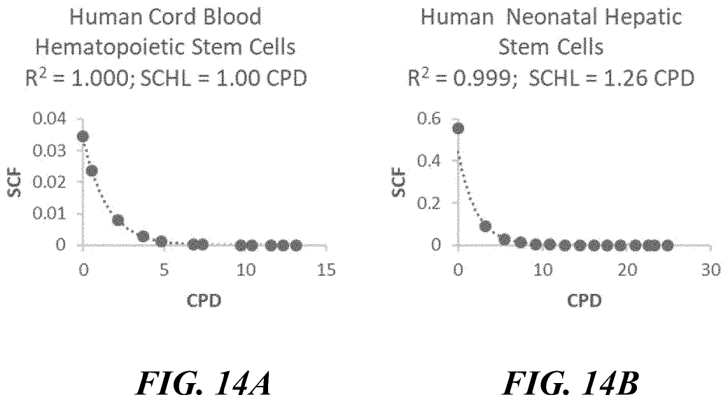

Specifically, the inventor discovered that mathematical graphs of SCF data (FIGS. 14A and 14B)—which are only available in the KSC counting computational simulation software—versus the simulated corresponding cumulative population doubling (CPD) data followed single-phase exponential decay kinetics with high statistical confidence (R²≥0.999). Previous to this invention, neither the inventor nor others trained in the art and science of tissue stem cell biology envisioned that such a precise biological relationship existed.

When the initial SCF of a primary tissue cell preparation is known, the SCF half-life (SCHL) makes it possible to calculate the SCF fraction of the cell preparation at any time in future cultures based on knowing the number of CPDs. CPD is a routine measure of the proliferative capacity of tissue cell preparations. The SCF at any future CPD (SCF_{CPD}) is calculated according to Formula E:

SCF_{CPD}, SCF at any future CPD,=SCF₀ Xₑ^{-(ln 2/HL)CPD} (Formula E),

where SCE₀=initial SCF and HL=half-life.

### Example 6

### Validation of Kinetic Stem Cell (KSC) Counting Algorithms for Rapid Quantification of Hematopoietic Stem Cells in Preparations for Stem Cell Medicine

Determination of the specific dosage of tissue stem cell (TSC) treatment products used in stem cell medicine continues to be a singular challenge. Herein is described a computational simulation method for routine, specific and accurate counting of vertebrate TSCs. The method quantifies TSCs based on their unique asymmetric cell kinetics, which are rate-limiting for TSCs' production of transiently-amplifying lineage-committed cells and terminally-arrested cells during serial cell culture. Because of this basis, the new method is now called kinetic stem cell (KSC) counting. Herein are validations of the specificity and clinical utility of KSC counting. First, herein demonstrates its quantification of the expected increase in the hematopoietic stem cell (HSC) fraction of CD34⁺-selected preparations of human mobilized peripheral blood cells, an approved treatment product routinely used for HSC transplantation therapies. Previously, the KSC counting technology was used to define new mathematical algorithms with potential for rapid determination of TSC-specific fraction without the need for serial culture. A second important HSC transplantation treatment, CD34⁺-selected umbilical cord blood (UCB) cells, was used to investigate for this prediction. Using only an input of simple population doubling time (PDT) data, the KSC counting-derived “Rabbit algorithms” can be used to rapidly determine the specific HSC fraction of CD34⁺-selected UCB cell preparations with a high degree of statistical confidence. These latest validations of KSC counting support its potential to meet the long-standing unmet need for a method to determine stem cell-specific dosage in stem cell medicine.

### Introduction

A long-standing need in stem cell medicine has been a convenient means for routine determination of the specific dosage of tissue stem cells (TSCs) in treatments, whether containing natural TSCs or genetically-engineered ones.¹⁻³ Stem cell medicine is not unlike pharmaceutical and biopharmaceutical medicine in its need for accurate quantification of the essential therapeutic agent in patient treatments.³ A convenient method for routine determination of TSC-specific dosage would improve progress in the manufacture of TSC medicinal products,⁴ progress in stem cell and stem-gene therapy clinical trials, and the quality of approved TSC treatments.^{3,5,6}

This unmet clinical need has persisted for more than half a century because, for most TSCs, molecular biomarkers that identify them exclusively have proven elusive.¹⁻³ One exception to this disappointment is skeletal muscle satellite stem cells that can be exclusively identified in vivo by their specific expression of the transcription factors Pax3 and Pax7.⁷ However, in vitro during cell culture for expansion, even these biomarkers lose their exclusivity for identifying only long-lived tissue renewing stem cells.⁸⁻¹⁰ More widely used biomarkers for hematopoietic stem cells (HSCs; i.e., CD34 and CD133)¹ and mesenchymal stem cells (MSCs; i.e., CD73 and CD90)¹¹ also identify committed progenitor cells (CPCs) as well, both in vivo and in vitro. There is a common misconception in stem cell medicine that these widely available biomarkers for characterizing HSC-containing and MSC-containing cell preparations can quantify these TSCs by flow cytometry. However, in fact, since these biomarkers also detect the more numerous CPCs produced by TSCs, which predominate in both freshly isolated and cultured tissue cell preparations, their determinations are erroneous for TSC fraction and dosage.²⁻⁴

In the case of HSCs, which are the only TSCs approved for routine stem cell medical treatments, a method has been described for determination of the HSC-specific fraction of hematopoietic cell preparations.^{3,12} However, this method has never been developed and approved for clinical practice. The method, the limiting-dilution SCID mouse repopulating cell (SRC) assay, has many challenging features that undermine its ability to specifically estimate the HSC fraction of hematopoietic tissue cell preparations.^{3,12} As a Poisson statistics estimation, the SRC assay requires as many as 50-60 mice to confidently quantify a single treatment sample; and 12-16 weeks are required to complete the assay. The SRC assay's dependency on the health and engraftment properties of the transplanted mice also confounds its quantification of HSC fraction with HSC engraftment efficiency. These requirements and features have conspired to yield an expensive, protracted, and unreliable assay that has proven impractical for routine and accurate quantification of the fraction and dosage of HSCs in hematopoietic transplantation treatments.³

We recently described a new method for accurate and specific determination of the fraction and dosage of mammalian TSCs, with a focus on therapeutic human TSCs.¹³ More recently, we have named the method kinetic stem cell (KSC) counting to reflect its underlying biological principle. KSC counting is a computational simulation method that quantifies TSCs based on their unique cell kinetics. In the absence of cell immortalization or cell transformation, primary mammalian tissue cells maintain their in vivo cell kinetics programs ex vivo in cell culture.¹⁴⁻¹⁶ These programs are TSC-based cell turnover units, which, although losing many in vivo cell differentiation properties, maintain terminal division arrest by committed cell lineages.¹⁷⁻¹⁹ When primary tissue cells are passaged for long periods of serial culture, the total cell output kinetics are limited by the number and proliferation kinetics of the TSCs in the culture. The KSC counting computational simulation software uses an underlying TSC-based turnover unit cell kinetics model to decipher from serial culture cell count data the rate-limiting TSC fraction and TSC kinetics.¹³

In the first report of KSC counting, we also described its application to define new mathematical algorithms with potential for rapid computation of the TSC-specific fraction (SCF) of mammalian cell cultures.¹³ Now, we describe further development of two related “Rabbit algorithms.” Rabbit population doubling time (PDT) algorithms require only the input of the PDT of cultured tissue cells to compute their SCF; and related Rabbit cumulative population doubling (CPD) algorithms require only an input of cultures' CPDs to compute their SCF. The present report details further validations of KSC counting and the rapid-counting Rabbit algorithms. Commercially supplied therapeutic preparations for human HSCs are used to show that KSC counting yields the expected enrichment of HSCs after CD34⁺ cell fractionation; and we confirm that rapid-counting Rabbit algorithms for CD34⁺ umbilical cord blood (UCB) HSCs accurately compute the HSC fraction of both parallel and secondary independent CD34⁺ UCB cell preparations. These validations continue to advance KSC counting as a first method for routine determination of the specific fraction and dosage of therapeutic TSCs for stem cell medicine.

### Materials and Methods

### Cells

Three independent lots of human CD34⁺-selected UCB cells from different anonymous donors were purchased from HemaCare-Charles River Laboratories, Northridge, Calif., USA (Cat#CBC34C-3) and pooled for the described analyses. The pooled cell samples were from two male and one female donor; and from one “Hispanic,” one “Caucasion,” and one “Other” donor. The cell viabilities of the thawed samples determined by trypan blue dye exclusion were respectively 97%, 96%, and 96%. The lots were pooled to produce a single uniform sample with sufficient cells for subsequent replicate validation analyses. Pooling also reduced the probability of donor-specific observations.

Peripheral blood preparations from anonymous G-CSF-mobilized donors were purchased from HemaCare-CRL (Cat#M009C-1) and Stemcell Technologies, Vancouver, BC, Canada (Cat#70049.4). Respectively, the traits for donor 1 were male, blood type A-, “Hispanic,” and 59 years old; and for donor 2 they were male, blood type O−, “Caucasian,” and 35 years old. The respective cell viabilities of the thawed samples were 95% and 92%.

CD34⁺-selected blood cells from anonymous G-CSF-mobilized donors were purchased from HemaCare-CRL (Cat#M34C-GCSF-1) and AllCells, Quincy, Mass., USA (Cat#mLP RegF CR CD34 PS 1M). Respectively, the traits for donor 1 were male, blood type O−, “Hispanic,” and 54 years old; and for donor 2, they were M, blood type A+, “Black/African American,” and 40 years old. The respective cell viabilities of the thawed samples were 91% and 97%.

### Serial Culture for KSC Counting

Suspension serial culture was performed in 24-well cell culture plates with 2 mLs culture medium at 37° C. in humified incubators with a 5% CO₂ atmosphere. All culture studies were conducted in StemSpan™ SFEM II culture medium (Stemcell Technologies, Cat#09655) supplemented with StemSpan™ CD34⁺ Expansion Supplement (Stemcell Technologies, Cat#02691) as specified by the supplier and 1% penicillin/streptomycin (Gibco, Cat#15140122).

The pooled CD34⁺ UCB cells were used to initiate two duplicate set of serial cultures in parallel as described later for mobilized peripheral blood cells (MPBCs). One duplicate serial culture set was passaged with 1:10 splits, and the other duplicate serial culture set was passaged with 1:5 splits. The remainder of the pooled cells were used to initiate three 12 mL cultures in 75-cm² T-flasks. The input cell numbers for the flask cultures were 7.4×10⁵, 1.1×10⁶, and 9.6×10⁵. After 73 hours of culture, the respective recovered cell numbers were 6.4×10⁶, 5.0×10⁶, and 6.0×10⁶. The respective cell viabilities of recovered cells were 92%, 92%, and 93%. The recovered cells from each T-flask culture were divided into 3 equal aliquots and cryopreserved in liquid nitrogen. The cryopreserved cells were designated as passage 1 (P1) cells; and subsequent cultures initiated with them were designated as P2 secondary serial cultures.

The initiating P1 CD34⁺ UCB cell serial cultures were initiated with 150,000 viable cells per 2.25 mL culture medium and performed in duplicate. Unfractionated MPBC cultures and CD34⁺-selected MPBC cultures were initiated with 100,000 viable cells per 2.25 mL culture medium and performed in duplicate. P2 CD34⁺ UCB secondary cultures were initiated with 100,000 viable cells per 2.25 mL culture medium and performed in triplicate. With the exception of the 1:5 split basis for one duplicate set for the P1 CD34⁺ UCB cell serial cultures, all cultures were passaged every 3 days with a 1:10 split basis. At each passage, the passage interval time was recorded with quarter-hour precision; and triplicate samples of collected culture cells were counted for trypan dye-excluding (live) and staining cells (dead) with a hemocytometer slide. Serial cultures were continued until either no increase in cell number was observed or no cells were detectable for two consecutive passages (i.e., terminal cell proliferation arrest or terminal cell dilution limit, respectively).

### KSC Counting Software Analyses

The Probabilistic Stem Cell Kinetics (PSCK), Random Input Factor Search (RIFS), and PDT:SCF algorithm programming and software for KSC counting have been described.¹³ In this report, the PSCK-RIFS software is renamed TORTOISE Test™ software; and the PDT:SCF algorithm discovery software is renamed the RABBIT Count™ software. Previously a manual program, the RABBIT Count™ software is now automated for high-density computations. Public access for use of the KSC counting software is available as a free calculator at https://asymmetrex.com/stem-cell-counting-center/. Formulae and calculators for PDT and CPD can be found at the same website.

All reported KSC counting analyses are based on 10 independent computer simulations of experimental CPD data from the described duplicate or triplicate serial cultures. Each optimal simulation results from 1000 searches by the TORTOISE Test™ software for sets of initial TSC, CPCs, and terminally-arrested cell fractions and cell kinetics factor values that can compute the observed experimental data based on a TSC-based turnover unit model.¹³ The mean simulation quality scores (SQS) for all reported analyses ranged from 0.004 to 0.23 (median=0.05), well below the ideal threshold SQS≤0.5.¹³

Initial cell kinetics factors determined by the TORTOISE Test™ software (See Table 6) were used to define Rabbit algorithms as described previously.¹³ The algorithms were derived with 10 independent computations of the experimental CPD data that gave simulations with fractional root-mean-squared error values (fRMSE) that were ≤0.10. fRMSE is the RMSE of a CPD data simulation compared to the experimental CPD data divided by the maximum value of the experimental CPD data.

Statistical and graphical analyses of KSC counting software outputs were performed with 2020 GraphPad Prism 9 for macOS software, version 9.0.0.

### Results

### KSC Counting Analyses of CD34⁺ UCB Cells

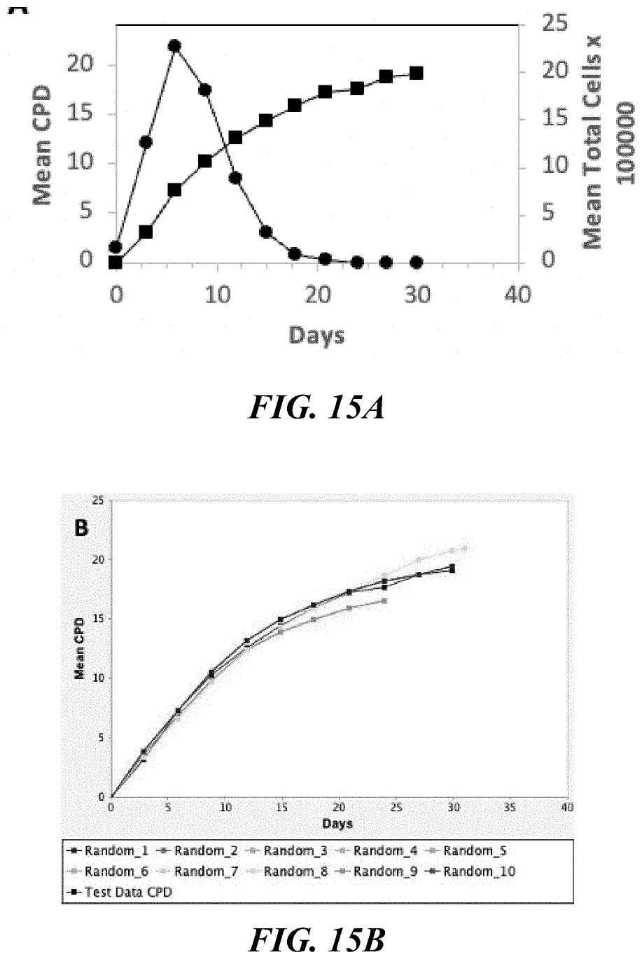

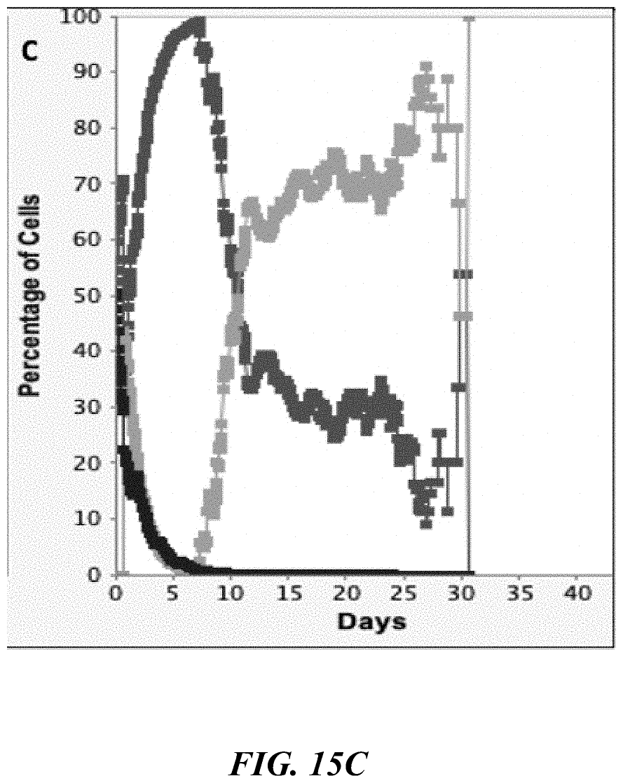

For this report, we evaluated commercially available human hematopoietic stem cell preparations that are currently widely used for HSC research and HSC transplantation medicine without the benefit of HSC-specific fraction or dosage. FIGS. 15A-15C provide the key elements of a TORTOISE Test™ software KSC counting analysis for CD34⁺ UCB cells. After about 10 serial passages over a period of 30 days, these cultures reach a terminal proliferation arrest, characteristic of all human primary tissue cell cultures.^{15,20} The total cell count data at each passage (FIG. 15A, circles) is transformed into CPD data (FIG. 15A, squares). The KSC counting TORTOISE Test™ software inputs several measured experimental culture factors and searches for unknown initial cell kinetics factors that together achieve a close simulation of the experimental CPD data (FIG. 15B).¹³ The search for initial cell kinetics factors requires that computed simulations match both the magnitude and the variance of the experimental CPD data (data not shown¹³). Among the initial cell kinetics factors discovered is the HSC-specific fraction of the initial cultured sample (See Table 6; CD34 UCB, SCF).

As presented in Table 6, the KSC counting TORTOISE Test™ software also allows discovery of additional important TSC-specific kinetics factors and cell kinetics factors specific for CPCs and terminally-arrested cells (data not shown). Using the PSCK module of the software (See Materials and Methods), these initial cell kinetics factors can be used to calculate the relative cell kinetics profiles for HSCs, CPCs, and terminally-arresting cells during the serial passage (FIG. 15C).

“Rabbit Algorithms” for Rapid Counting of Human HSCs from Different Sources

Table 6 summarizes initial cell kinetics factors determined by TORTOISE Test™ KSC counting analyses of three different types of cell preparations containing HSCs from three different commercial suppliers. The preparations include the CD34⁺ UCB cells described earlier, G-CSF-mobilized unfractionated peripheral blood cells, and G-CSF-mobilized CD34⁺-selected peripheral blood cells. Importantly, all CD34⁺-selected cell sources had a significantly higher HSC-specific fraction defined by KSC counting (Table 5, SCF). Although the HSC fraction of the two different donor sources of unfractionated MPBCs differed significantly, they were both significantly lower than the HSC fraction of all CD34⁺-selected preparations. In contrast, all three preparations for CD34⁺-selected cells—from both UCB and two different sources of adult MPBC donors—had higher statistically equivalent HSC fractions. The degree of cell enrichment from the suppliers was not available, but the mean increase in HSC fraction for the CD34⁺-selected MPBC preparations compared to unfractionated MPBCs was 9.5-fold (p<0.01 by the unpaired, two tailed Student's t-test).

Numerical differences were noted among the five evaluated sources of HSCs for initial cell kinetics factors determine by the TORTOISE Test™ KSC counting software analysis. However, only a few differences were statistically significant and only for some comparisons. These included a lower rate of HSC symmetric self-renewal by one source of unfractionated MPBCs (Table 5, MPBC, HemaCare, SC SSR); a lower rate of CPC death by the same commercial source of unfractionated MPBCs and CD34⁺ UCB cells (Table 5, CPC Death Rate); and a generally greater number of CPC divisions before terminal division arrest by CD34⁺ UCB cells (Table 6, TDN).

TABLE 6
Comparison of Initial Cell Kinetics Factors Determined by KSC
Counting of Commercial Human Hematopoietic Cell Preparations¹

| | Cell Supplier: | | | | | | | | | |
|---|---|---|---|---|---|---|---|---|---|---|
| | CD34 UCB² | | MPBC | | | | CD34 MPBC | | | |
| | HemaCare | | HemaCare | | Stemcell Tech | | HemaCare | | AllCells | |
| | (Mean; 95% Confidence Interval) | | | | | | | | | |
| Hematopoietic Stem | | | | | | | | | | |
| Cell Factors | | | | | | | | | | |
| SCF | 0.72; | 0.58-0.85 | 0.02; | 0.01-0.03 | 0.15; | 0.06-0.24 | 0.78; | 0.61-0.96 | 0.83; | 0.76-0.91 |
| SC SSR | 0.40; | 0.23-0.57 | 0.13; | 0.06-0.20 | 0.39; | 0.17-0.62 | 0.51; | 0.30-0.71 | 0.35; | 0.18-0.52 |
| SC Asym CC Time | 8.4 h; | 4.4-12 | 9.9 h; | 7.4-12 | 18 h; | 10-25 | 8.9 h; | 5.5-12 | 12 h; | 3.4-20 |
| SC Sym CC Time | 24 h; | 16-31 | 9.1 h; | 6.2-12 | 13 h; | 6.8-19 | 19 h; | 11-28 | 19 h; | 12-27 |
| SC Death Rate | 0.07; | 0.01-0.13 | 0.21; | 0.09-0.32 | 0.28; | 0.09-0.47 | 0.15; | 0.02-0.28 | 0.18; | 0.05-0.30 |
| SCFHL | 1.37; | 1.30-1.44 | 2.13; | 0.21-id³ | 1.10; | 0.79-1.67 | 3.16; | 2.26-4.74 | 1.54; | 1.43-1.66 |
| Committed Progenitor | | | | | | | | | | |
| Cells Factors | | | | | | | | | | |
| CPC CC Time | 24 h; | 22-26 | 12 h; | 10-15 | 14 h; | 10-18 | 20 h; | 12-28 | 17 h; | 12-23 |
| CPC Death Rate | 0.01; | −0.01-0.04 | 0.07; | 0.03-0.12 | 0.45; | 0.26-0.64 | 0.33; | 0.07-0.59 | 0.19; | 0.01-0.38 |
| TDN | 10; | 9.5-11 | 5; | 3-7 | 5; | 2-8 | 6; | 2-11 | 7; | 4-10 |

¹Determinations from N = 10 simulations.
²CD34 UCB, CD34⁺ -selected umbilical cord blood cells; MPBC, mobilized peripheral blood cells; SCF, TSC-specific fraction; SC SSR, stem cell symmetric self-renewal rate, the fraction of stem cells that divide symmetrically in any generation; Asym CC Time, cell cycle time in hours for asymmetrically dividing TSCs; Sym CC Time, cell cycle time in hours for symmetrically dividing TSCs; CPC CC Time, cell cycle time in hours for committed progenitor cells; TDN, turnover division number, the number of cell generations before CPC lineages terminate in non-dividing, terminally-arrested cells; Death Rate, fraction of cells that die in any cell generation; SCFHL, TSC fraction half-life in units of CPD, cumulative population doublings.
³id, indeterminate interval boundary.

There are three basic determinants of changes in the SCF during serial culture. These are the rate at which TSCs symmetrically self-renew, the rate at which TSCs die or are lost from the culture by other mechanisms (e.g., differentiation or cell fusion), and the rate at which they are diluted by other cells produced in cultures—by either the asymmetric division of stem cells or the division of other cell types in the culture. As shown in FIG. 15C (blue trace), the TORTOISE Test™ software uses the simulation-derived estimates of these determinants to render a computation of how the SCF changes with serial passage. All five sources of HSCs showed the previously described characteristic decline in SCF with continued passage.¹³

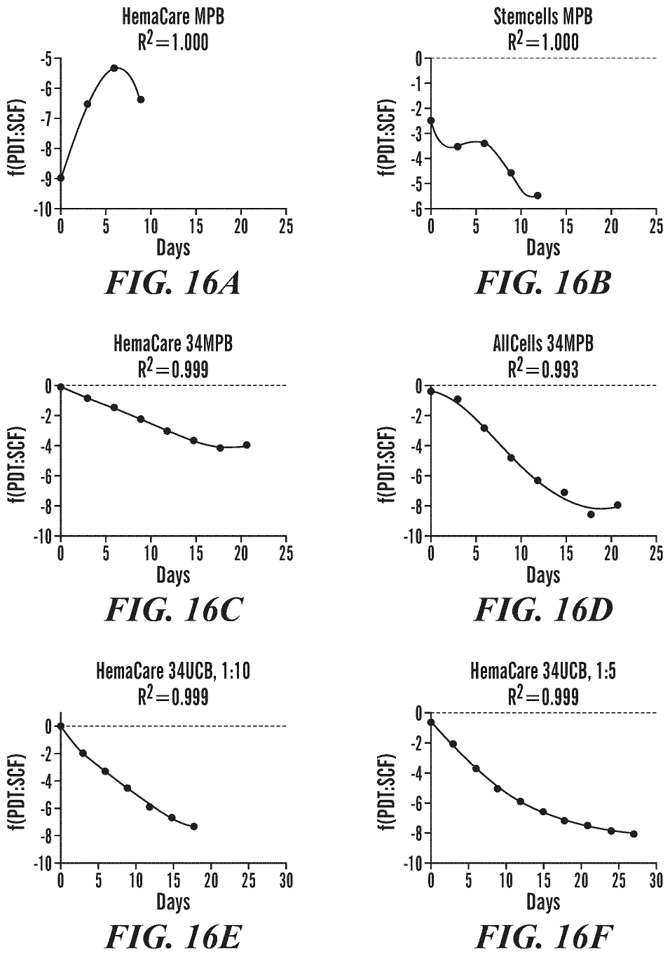

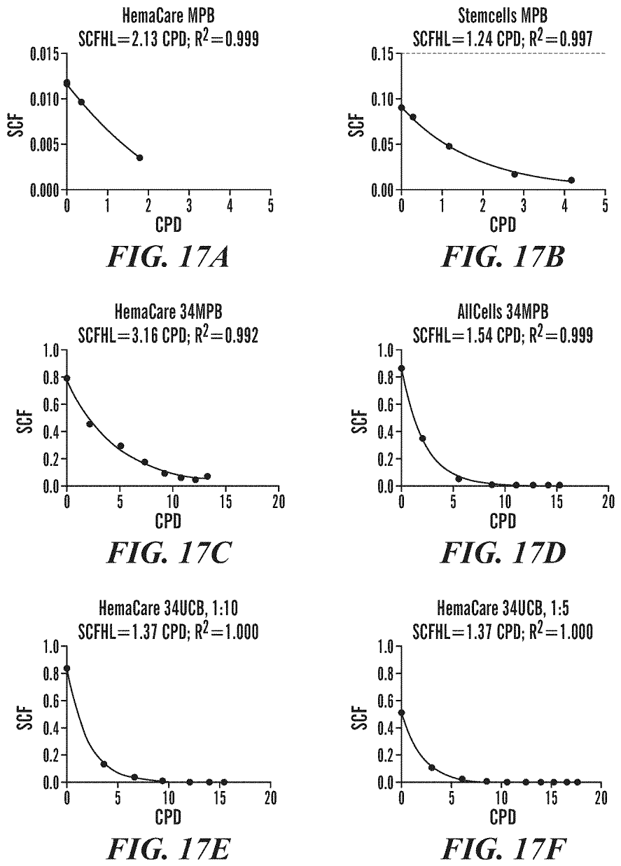

The newly automated RABBIT Count software was used, as described previously, to relate the simulated SCF of cultures with passage time to the simulated 72-hour PDTs of cultures with passage time.¹³ These computations yield two different types of “Rabbit algorithms”. Rabbit PDT algorithms (FIGS. 16A-16F) are proposed to be able to calculate the SCF of secondary serial cultures of the same source and culture conditions of the original by inputting the day of serial culture and the experimental PDT of the preceding 72-hour period. Rabbit CPD algorithms (FIGS. 17A-17F) are a simple mathematical transform of the PDT algorithms that allows computation of the SCF of similar secondary cultures directly from an input of only their number of CPDs. The CDP algorithms yield a novel TSC kinetics parameter called the stem cell fraction half-life (SCFHL). The SCFHL defines the number of CPDs required for a 50% decrease in a culture's SCF.

The general qualities of the Rabbit PDT algorithms reflected the main biological distinctions of the examined HSC preparations. As shown in FIGS. 16A-16F, though quantitatively distinctive, the Rabbit PDT algorithms for the CD34⁺-selected cell preparations were qualitatively similar in mathematical form, whether originating from UCB cells (FIGS. 16E and 16F) or adult donor peripheral blood (FIGS. 16C and 16D). In contrast, the PDT algorithms for HSCs in unfractionated NIPBC preparations (FIGS. 16A and 16B) were quantitatively and qualitatively distinctive from the CD34⁺-selected sources, as well as from each other.

The CPD algorithms for all examined HSC sources quantified the characteristic decline in HSC fraction with increasing serial culture passages. The computed SCFHLs ranged from 1.10 CPDs to 3.16 CPDs. It is noteworthy that the SCFHL of one source of CD34⁺-selected IVIPBCs (Table 6, CD34 NIPBC, HemaCare) was significantly greater than the SCFHLs of all other HSC sources. This difference can be attributed to a higher rate of HSC symmetric self-renewal (Table 6, SC SSR), a lower rate of cell death for HSCs (Table 6, SC Death Rate), and a substantial rate of cell death for CPCs (Table 6, CPC Death Rate) in this source.

### KSC Counting Rabbit Algorithm Validations

Two studies were performed to evaluate the ability of Rabbit algorithms to confidently calculate the SCF of secondary cultures of the same tissue cell source and culture conditions with an input of experimental 72-hour PDT data. For the first evaluation, the same pooled source of CD34⁺ UCB cells (See Materials and Methods) shown in FIGS. 15A-15C for serial culture with a 1:10 split basis was serially cultured in parallel with a 1:5 split basis. None of the initial cell kinetics factors (shown in Table 6) defined by the TORTOISE Test™ KSC counting software analysis were significantly different between the 1:10 split basis and the 1:5 split basis (data not shown).

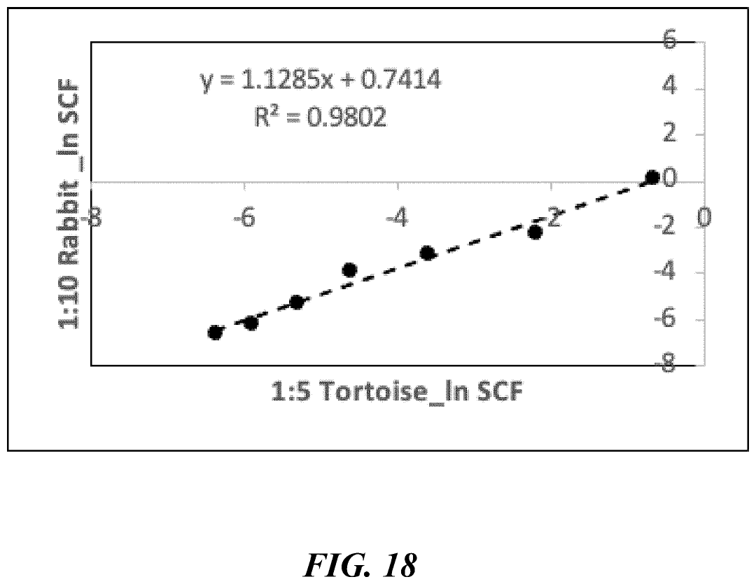

Consistent with this finding, KSC counting based on the two different split bases yielded very similar PDT algorithms (Compare FIGS. 16E and 16F) and equal SCFHLs (1.37 CPDs; Compare FIGS. 17E and 17F). Inputting the experimental 72-PDT data from the 1:5 split basis serial culture into the PDT algorithm determined with the 1:10 split basis serial cultures yielded SCF calculations that had a high degree of agreement with the independent TORTOISE Test™ software determinations of the SCF during serial culture with a 1:5 split basis (FIG. 18; R²=0.980). The 1:5 split basis serial cultures extended for three passages beyond the final passage of the serial cultures with the 1:10 split basis (Compare FIGS. 16F and 16E). This difference in the time of terminal cell proliferation arrest is predicted by the TSC-based tissue cell turnover unit model, which is the biological principle for KSC counting technology. The SCF fractions projected by the 1:10 split basis Rabbit PDT algorithm showed no discernable agreement with the TORTOISE Test™ software determinations of the SCF of these experimentally uncovered passages (data not shown).

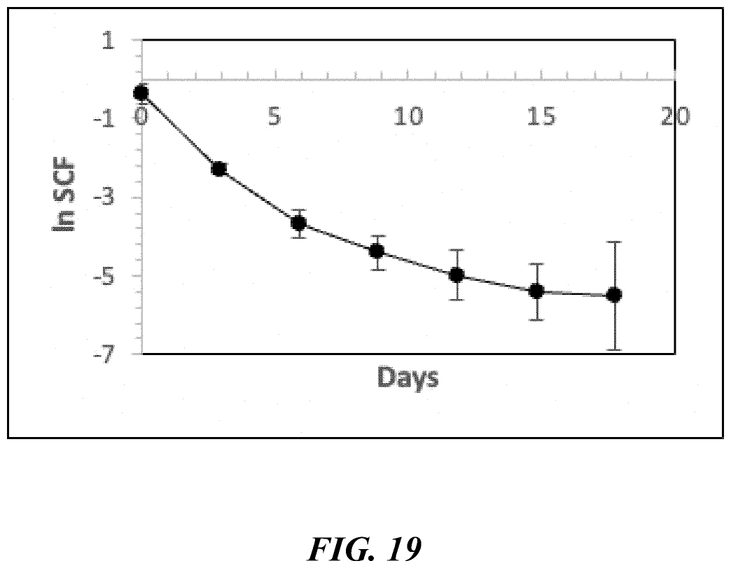

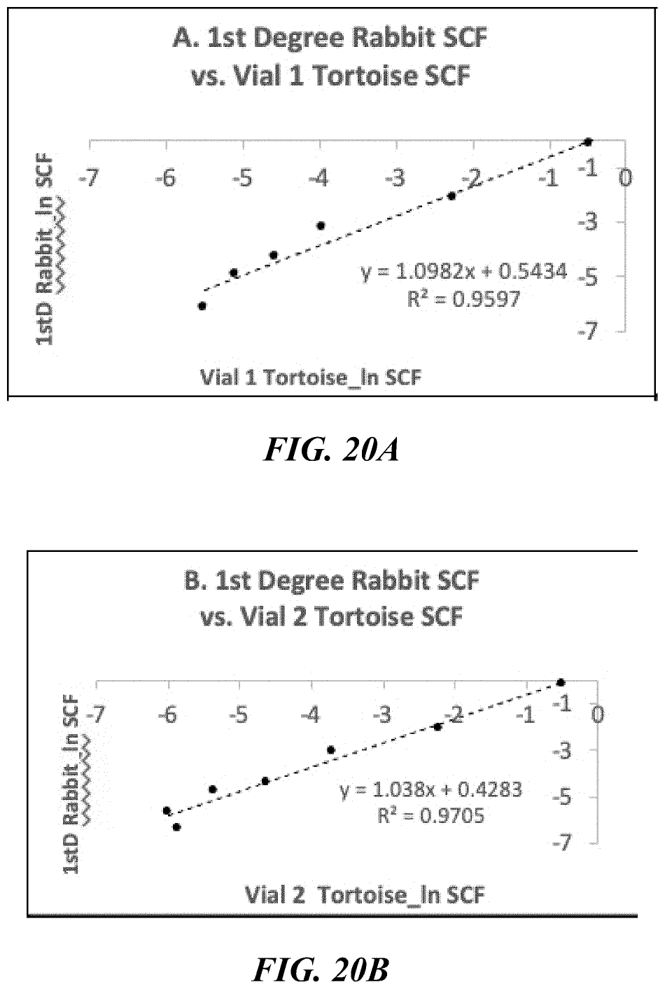

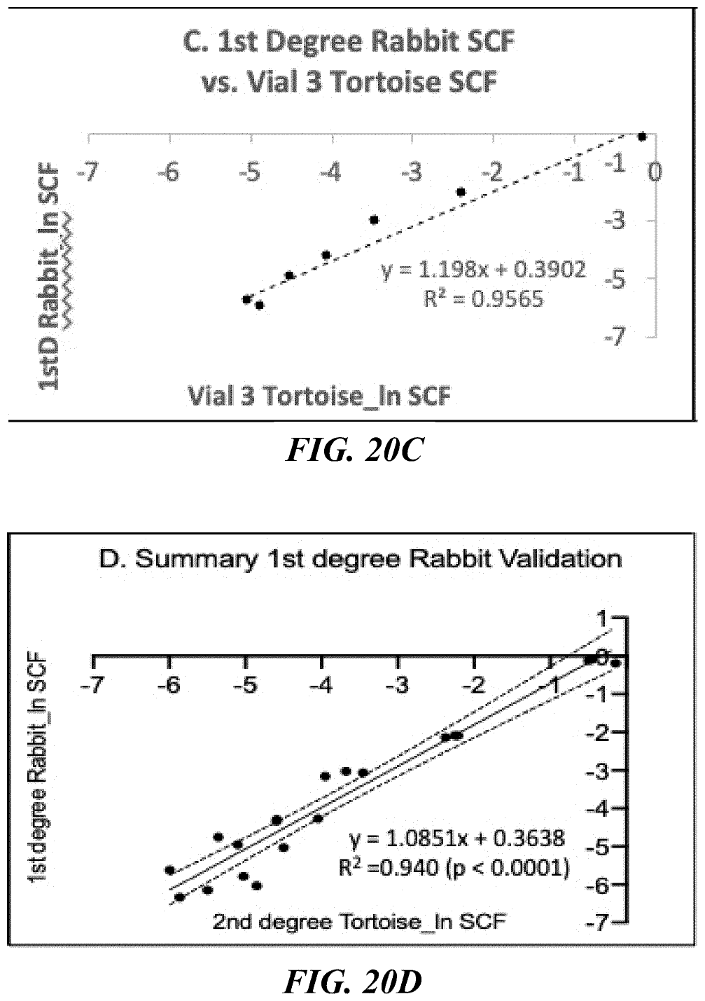

For the second evaluation, in parallel with the serial cultures described for the data in FIGS. 15A-15C, three independent bulk cultures were initiated in the same type of culture medium and cultured in parallel for 72 hours. The cells in the resulting 72-hour cell cultures were harvested and cryopreserved (See Material and Methods for details). Several weeks later, one vial of cells corresponding to each of the three independent cryopreserved parallel cell cultures (Vials 1-3 in FIGS. 19-20D) were thawed and used to perform three secondary serial cultures (each in triplicate) for three respective secondary TORTOISE Test™ KSC counting analyses. These passage 2 (P2) secondary cultures were also passaged with a 1:10 split basis. FIG. 19 shows the mean SCF determinations for the three independent secondary TORTOISE Test™ analyses. The three independent sets of SCF determinations show a high degree of consistency until the final passages, where variability in cell counting data increases because of low total cell numbers.

To evaluate the ability of the original 1^{st} degree Rabbit PDT algorithm to accurately determine the SCFs for the secondary cultures, the mean experimental 72-hour PDT data from each secondary serial culture were input into the 1st degree Rabbit PDT algorithm to calculate SCFs during serial passaging. The results of this evaluation are presented in FIGS. 20A-20D. The 1st degree Rabbit PDT algorithm yielded SCF determinations with a high degree of accuracy as judged by their correlation with the SCF values determined by the independent secondary TORTOISE Test™ analyses. The correlation coefficients range from 0.957 to 0.971 (FIGS. 20A-20C); and the correlation coefficient for a combined analyses including all three secondary cultures was 0.940, with statistical confidence at the level of p<0.0001 (FIG. 20D).

### Discussion

In this study, the inventor reports results from the examination of three commercially-supplied sources of human HSCs, used in stem cell medical treatments, with a recently developed technology designed for convenient, routine determination of TSC-specific fraction and dosage. The focus of the presented analyses was human HSCs, but the new technology, KSC counting, has general application for TSCs in many different mammalian tissues of interest for research and medicine. Therapeutic human HSCs were the quintessential choice as the focus for continued KSC counting investigation and validation. Even though widely-used, approved HSC transplantation treatments are currently practiced without HSC-specific dosing, patients would immediately benefit from this improvement. In particular, KSC counting provides a long-needed effective basis for determining the potency of UCB units, which have a failure rate of 18-24% attributable to insufficient HSC dosage.²¹

The presented studies had two main validation goals for KSC counting. Previously, we used published serial culture data to demonstrate the ability of the KSC counting technology to detect and quantify the well-described increase in the HSC-fraction of CD34⁺-selected human hematopoietic tissue cell populations.¹³ In the present report, we demonstrated this validation directly with two independent sources of commercially-supplied hematopoietic cells. The second validation goal was to confirm that previously described rapid counting mathematical algorithms, herein named “Rabbit algorithms”, were able to calculate the SCF from the PDT of cultures with a high degree of statistical confidence. The presented results lead us to conclude that both validations goals were achieved.

Although the HSC fractions determined by KSC counting of two independent donor samples of unfractionated MPBCs seem reasonable with our range of expectations (Table 5; 2% and 15%), all CD34⁺-selected preparations had high HSC fractions that were not in the range of our expectations based on prior experience (Table 5; 72%, 78%, and 83%). These HSC fractions are also significantly higher than the fractions determined earlier with KSC counting based on published serial culture data for CD34⁺-selected UCB cells (8.0% and 0.24% with a 20-fold enrichment by CD34⁺ selection)¹³. Therefore, the present results are specific to the newly examined samples and not a general feature of the KSC counting method. This feature may have been missed in the past, because the HSC fraction rapidly declines with culture of these products, especially with the standard supplementation with growth factors that promote CPC proliferation and potentially HSC death.

The major value of KSC counting is convenient, routine determination of the specific fraction and specific dosage of therapeutic TSCs, like HSCs in this report, and others as well like MSCs, liver stem cells, and corneal stem cells.¹³ In the reported studies, we increased experience with the properties of mathematical algorithms that were defined previously for the purpose of more rapid counting of TSCs. Intrinsic to their derivation, the Rabbit PDT algorithms quantitatively and qualitatively manifest cell-autonomous TSC kinetics properties and secondary effects on SCF due to the cell kinetics of other tissue cell types present in tissue cell cultures. This signature character of the algorithms was evident from the similarity of PDT algorithms for three different sources of CD34⁺-selected hematopoietic cell preparations. Such fractionated cell populations are expected be more homogeneous for HSCs and CPCs. However, unfractionated mobilized peripheral blood cells are significantly more heterogeneous for cell type and cell proliferation kinetics. In fact, in these preparations the modeled compartments for transiently dividing “CPCs” and “terminally-arrested cells” include all hematopoietic cell differentiation lineages. The greater cell heterogeneity was associated with PDT algorithms that differed substantially quantitatively and in mathematical form between different donors and from CD34⁺-selected cells.

In the present report, inventor describes the development of a new type of algorithm, Rabbit CPD algorithms. CPD algorithms can be used to define a new quantitative TSC kinetics parameter, the SCFHL. SCFHL is a simple new state function parameter for TSCs that reflects the interplay of the several complex cell kinetics factors that intersect to determine changes in SCF during serial cell culture.

Beyond the major advance of providing a practical method for routine determination of TSC-specific fraction and dosage, KSC counting reveals a number of human tissue cell kinetics parameters that were previously inaccessible. The development and validation of rapid-counting Rabbit algorithms illustrates the power for the KSC counting TORTOISE Test software to provide new ways of measuring and monitoring changes in new potential critical quality attributes (CQAs) for cell and tissue biomanufacturing. Several of the initial cell kinetics factors isolated for interrogation for the first time by KSC counting (Table 5) could prove to be highly advantageous for breaching long-standing barriers to stem cell medicine goals like expanding therapeutic TSCs.^{14,16,22}

### CONCLUSIONS

Kinetic stem cell (KSC) counting is a recently developed technology for quantification of the specific fraction and dosage of therapeutic tissue stem cells (TSCs), like hematopoietic stem cells (HSCs). Further validation of the specificity and accuracy of KSC counting was achieved by demonstrating the technology's ability to quantify the enrichment of HSCs in commercially-supplied CD34⁺-selected mobilized peripheral blood cell preparations. New “Rabbit algorithms” are described that allow rapid quantification of HSC fraction and dosage based on either the population doubling time (PDT) or the cumulative population doublings (CPD) of cultured hematopoietic cell preparations. The Rabbit CPD algorithms provide a novel TSC kinetics parameter called the stem cell fraction half-life (SCFHL), which is the number of culture CPDs required for the TSC-specific fraction to decrease by 50%. The SCFHL of examined commercial human HSC sources ranged from 1.10 CPDs to 3.16 CPD. The Rabbit algorithms exhibited a high degree of confidence for calculating the HSC fraction of parallel and secondary preparations of CD34⁺ umbilical cord blood (UCB) cell preparations, with R² values ranging from 0.940 to 0.980. These latest characterizations of the new KSC counting technology further validate its potential to be the long-needed method for routine quantification of the specific fraction and specific dosage for TSCs for stem cell research and stem cell medicine.

### REFERENCES

1. Ivanovic Z. Hematopoietic stem cells in research and clinical applications: The “CD34 issue”. World J Stem Cells 2010; 2:18-23.
2. Sherley J L. Principles for targeting adult tissue stem cells to achieve durable gene and gene-editing therapeutics. In: Appasani K, ed. Genome Editing and Engineering: From Talens, Z F N, and CRISPRs to Molecular Surgery. Cambridge: Cambridge University Press, 2018:285-300.
3. Sherley J L. Dose determination for stem cell medicine: a need whose time has come. In: Atala A, Cetrulo C, Cetrulo K, Murphy S V, Taghizadeh R, eds. Perinatal Stem Cells: Research and Therapy. Amsterdam: Elsevier, 2018:383-397.
4. Sherley J L. (2018) Stem cell therapy: resolving the mismatch. Pharmaceutical Manufacturing 2018; March:38-41.
5. Sherley J L. Achieving dose standardization for the stem cell clinical trials industry. Operations 10:03, May 31, 2018; http://www.clinicaltrialsarena.com/news/operations/achieving-dose-standardization-for-the-stem-cell-clinical-trials-industry-6172126.
6. Sherley J L. Why don't stem-gene clinical trial sponsors count their stem cells. Arena Intl. Events Grp. H1 Virtual Events: Review and Summary Handbook 2021; 14-16. https://content.yudu.com/web/442ay/0A447nd/H1VE-RSH21/html/index.html?page=2&origin=reader.
7. Kassar-Duchossoy L, Giacone E, Gayraud-Morel B et al. Pax3/Pax7 mark a novel population of primitive myogenic cells during development. Genes & Devel. 2005; 19:1426-1431.
8. Yin H, Price F, Rudnicki M A. Satellite cells and the muscle stem cell niche. Physiol. Rev. 2013; 93:23-67, doi: 10.1152/physrev.00043.2011.
9. Huh Y H, Noh M, Burden F et al. Use of sparse feature bioinformatics to identify a novel pattern-specific biomarker for counting asymmetrically self-renewing distributed stem cells. Stem Cell Res. 2015; 14:144-154. 10.1016/j.scr.2014.12.007.
10. Homma S, Chen J C, Rahimov F et al. A unique library of myogenic cells from facioscapulohumeral muscular dystrophy subjects and unaffected relatives: family, disease and cell function. Eur. J. Hum. Genet. 2012; 20:404-410.
11. Ramos T L, Sanchez-Abarca L I, Mutión S. et al. MSC surface markers (CD44, CD73, and CD90) can identify human MSC-derived extracellular vesicles by conventional flow cytometry. Cell Commun. Signal 2016; 14:2, doi: 10.1186/s12964-015-0124-8.
12. Purton L E, Scadden D T. Limiting factors in murine hematopoietic stem cell assays. Cell Stem Cell 2007; 1:263-270.
13. Dutton R, Abdi F, Minnetyan L et al. A computational technology for specific counting of perinatal and postnatal human tissue stem cells for transplantation medicine,” OBM Transplantation 2020; 4:24; doi:10.21926/obm.transplant.2003117.
14. Pare J-F, Sherley J L. Biological principles for ex vivo adult stem cell expansion. In: Schatten G, ed. Current Topics in Developmental Biology. Vol. 73. San Diego: Elsevier Inc.,2006: 141-171.
15. Rambhatla L, Bohn S A, Stadler P B et al. Cellular senescence: ex vivo p53-dependent asymmetric cell kinetics. J. Biomed. Biotech. 2001; 1:27-36.
16. Merok J R, Sherley J L. Breaching the kinetic barrier to in vitro somatic stem cell propagation. J. Biomed. Biotech. 2001; 1:24-26.
17. Sherley J L. New cancer diagnostics and therapeutics from a 9^{th} “hallmark of cancer”: symmetric self-renewal by mutated distributed stem cells. Expert Rev. Mol. Diagn. 2013; 13:797-810.
18. Potten C S, Morris R J. Epithelial stem cells in vivo. J. Cell Sci. Suppl. 1988;10:45-62.
19. Loeffler M, Potten C S. Stem cells and cellular pedigrees—a conceptual introduction. In: Potten C S, ed. Stem Cells. San Diego: Harcourt Brace & Co., 1997:1-28.
20. Hayflick L. The limited in vitro lifetime of human diploid cell stains. Exp. Cell Res. 1965; 37:614-636.
21. Olsson R, Remberger M, Schaffer M et al. Graft failure in the modern era of allogeneic hematopoietic SCT. Bone Marrow Tramp. 2013; 48:537-543.
22. Sherley J L. Accelerating progress in regenerative medicine by advancing distributed stem cell-based normal human cell biomanufacturing. Pharm. Anal. Acta. 2014; 5: 86. doi: 10.4172/2153-2435.1000286.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method for determining the fraction of tissue stem cells present in a tissue sample, the method comprising:
(a) culturing a heterogeneous population of cells from a specific tissue comprising tissue stem cells, transiently amplifying committed progenitor cells and terminally differentiated non-dividing cells;
(b) serially passaging said population of cells wherein the total number of live and dead cells in the population of cells are counted;
(c) processing the total cell count data with a model to determine the stem cell fraction (SCF) of the population of cells throughout the periods of the serial passaging;
(d) calculating the population doubling times (PDT) for said tissue cells during non-saturated or non-confluent periods of the serial passaging; and
(e) quantifying the number of tissue stem cells in the subsequent tissue samples of the same type based on (d), without serial passaging.

2. The method of claim 1, wherein the PDT is determined by the following Formula A:
$$PDT = \frac{{t}_{2} - {t}_{1}}{ln ( \frac{{N}_{2}}{{N}_{1}} )} ln 2 \qquad ( Formula A )$$
where t₁=initial starting time, t₂=final time, N₁=the initial cell number, and N₂=the final cell number.

3. The method of claim 1, wherein the SCF is determined by one of the following derivative Formulas B-D:
ISCF=PDT (mean quotient of SCF/PDT) (Formula B);
SCF=e^{-(mPDT+b)} (Formula C); or
SCF=PDTe^{(at\hat{ }3+bt\hat{ }2+ct+d)} (Formula D),
where t=time.

4. The method of claim 1, wherein the cells are passaged every 12 hours or more, 24 hours or more, 48 hours or more, 72 hours or more, or 96 hours or more.

5. The method of claim 1, wherein the cells are passaged on an irregular schedule based on when they achieve saturation density or confluence.

6. The method of claim 1, wherein a constant fraction of cells is serially passaged.

7. The method of claim 1, wherein a constant number of cells are serially passaged.

8. The method of claim 1, wherein the PDT is determined in vitro or by in vivo imaging techniques.

9. The method of claim 1, wherein the PDT is determined by using a counting method selected from the group consisting of: counting cells, an absorbance assay, a turbidity assay, weighing cells, a fluorescent assay, and combinations thereof.

10. The method of claim 1, wherein the cells are serially passaged until the number of total cells after two consecutive passages does not increase.

11. The method of claim 1, wherein the processing time is determined by a computer processing system.

12. The method of claim 1, wherein the cells are processed until a predetermined number of tissue stem cells are present in the sample.

13. The method of claim 1, wherein the cells are a vertebrate population of cells.

14. The method of claim 1, wherein the cells are a mammalian population of cells.

15. The method of claim 1, wherein the cells are a human population of cells.

16. The method of claim 1, wherein the culturing is selected from the group consisting of: 3-dimensional cell culture; suspension cell culture; adherent cell culture; microcarrier cell culture; and any combination thereof.

17. The method of claim 1, further comprising contacting the cells with an agent.

18. The method of claim 1, wherein the cells are cultured in hypoxic conditions.

19. The method of claim 1, wherein the tissue stem cells are separated from initial transiently amplifying committed progenitor cells and terminally differentiated non-dividing cells.

20. The method of claim 1, wherein the tissue stem cells are used to treat an individual.

21. The method of claim 1, further comprising administering to a subject in need thereof an appropriate amount of stem cells based on the stem cell fraction (SCF).

22. The method of claim 21, wherein the stem cells have been genetically modified.

23. The method of claim 22, wherein the genetic modification is selected from the group consisting of: deleting gene sequences, inserting gene sequences, editing gene sequences in the nuclear genome, editing sequences in mitochondrial genomes.

24. The method of claim 21, wherein the stem cells are administered as a therapeutic agent for reducing the signs and symptoms of diseases, disorders, and injuries or for providing cosmetic changes in an individual.

25. A method of treating a subject in need thereof with a population of stem cells, the method comprising:
(a) determining the stem cell fraction present in a tissue sample according to the method of claim 24; and
(b) administering to the subject in need thereof a dosage of stem cells based on the stem cell fraction (SCF).

26. The method of claim 25, wherein the subject in need thereof has or is suspected of having a disease, disorder, or injury.

27. The method of claim 25, wherein the disease, disorder, or injury is selected from the group consisting of effects on organs and tissues such as, but not limited to: lungs, heart, blood vessels, blood, liver, pancreas, muscle, bones, joints, eyes, central and peripheral nervous systems.

28. The method of claim 25, wherein the stem cells have been genetically modified.

29. The method of claim 28, wherein the genetic modification is selected from the group consisting of: deleting gene sequences, inserting gene sequences, editing gene sequences in the nuclear genome, editing sequences in mitochondrial genomes.
